(19) United States
(12) Patent Application Publication
(10) Pub. No.: US 2024/0167051 A1
(43) Pub. Date: May 23, 2024

(54) CORN TRANSGENIC EVENT MON 95379 AND METHODS FOR DETECTION AND USES THEREOF

(71) Applicant: Monsanto Technology LLC, St. Louis, MO (US)

(72) Inventors: Heather M. Anderson, Wildwood, MO (US); Sarah L. Brown, Weldon Spring, MO (US); Renato A. Carvalho, St. Louis, MO (US); Ancideriton A. Castro, St. Louis, MO (US); Katherine M. Dunkmann, St. Louis, MO (US); Adam J. Evans, St. Louis, MO (US); Stanislaw Flasinski, Ballwin, MO (US); Cara L. Griffith, Catawissa, MO (US); Tianxiang Shen, St. Louis, MO (US); Todd R. Smith, St. Louis, MO (US); Heidi M. Windler, St. Louis, MO (US)

(21) Appl. No.: 18/497,852

(22) Filed: Oct. 30, 2023

Related U.S. Application Data
(62) Division of application No. 17/459,303, filed on Aug. 27, 2021, now Pat. No. 11,845,949.
Application No. 17/459,303 is a division of application No. 16/525,278, filed on Jul. 29, 2019, now Pat. No. 11,198,887.
(60) Provisional application No. 62/711,810, filed on Jul. 30, 2018.

(51) Int. Cl. C12N 15/82; C07K 14/325; C12N 15/10; C12Q 1/6895

(52) U.S. Cl. CPC C12N 15/8286; C07K 14/325; C12N 15/102; C12N 15/8274; C12Q 1/6895; C12Q 2600/13; Y02A 40/146

(57) ABSTRACT

The invention provides a transgenic corn event MON 95379, plants, plant cells, seeds, plant parts, progeny plants, and commodity products comprising event MON 95379. The invention also provides polynucleotides specific for event MON 95379 and methods for using and detecting event MON 95379 as well as plants, plant cells, seeds, plant pails, progeny plants, and commodity products comprising event MON 95379.

### REFERENCE TO RELATED APPLICATION

This application claims the benefit of U.S. provisional application No. 62/711,810, filed Jul. 30, 2018, which is herein incorporated by reference in its entirety.

### INCORPORATION OF SEQUENCE LISTING

The sequence listing contained in the file named MONS463US_ST25 is 74.7 kilobytes (measured in Microsoft Windows®), was created on Jul. 25, 2019, is filed herewith by electronic submission, and is incorporated by reference.

### FIELD OF THE INVENTION

The invention relates to recombinant DNA molecules present in and/or isolated from corn event MON 95379. The invention also relates to transgenic corn plants, plant parts, and seeds, cells, and agricultural products containing corn event MON 95379, as well as methods of using the same and detecting the presence of corn event MON 95379. Transgenic corn plants, parts, seeds and cells containing corn event MON 95379 DNA exhibit resistance to insect infestations in the family Lepidoptera.

### BACKGROUND OF THE INVENTION

Corn (Zea mays) is an important crop and is a primary food source in many areas of the world. The methods of biotechnology have been applied to corn for improvement of the agronomic traits and quality of the product. One such agronomic trait is insect resistance, which is accomplished through the expression of heterologous insect toxins, also known as transgenes, inserted into the genome of the corn plant.

The expression of such transgenes in a transgenic plant, plant part, seed or cell may be influenced by many different factors, including the elements used in the cassettes driving transgene expression and the interaction of those elements within a cassette. This is complicated further for a transgenic insertion containing two or more expression cassettes, with each expression cassette having a transgene conferring a separate trait, also known as a multi-gene transgenic event. A commercially useful multi-gene transgenic event requires that each of the transgenes in the transgenic insertion express in the manner necessary for each trait. To achieve this, individual expression cassettes first are designed and tested in plants, and the expression cassettes that show the best insect activity, while not resulting in negative phenotypes due to expression, are selected for each trait. Next, the selected expression cassettes for one trait are combined with the selected expression cassettes for the other trait into a single construct. Multiple constructs are designed using different orientations to provide the best resistance and prevent the occurrence of negative phenotypes or negative agronomics, such as lower yield. The constructs are tested to ensure all the expression cassettes function well together and each transgene is properly expressed. Then, the selected combination and orientation of expression cassettes is used as a single transgenic insert to produce hundreds transgenic events, each event being the result of the random insertion of the construct in a different genomic location.

Each transgenic event is unique in its molecular profile and chromosomal insertion point. Because of the variability involved in event creation, each unique event must be analyzed through multiple generations of plants—in each step assessing the molecular characterization, protein expression efficacy, and agronomics—to select a superior event for commercial use. The performance of an event in a transgenic plant, plant part, seed or cell, and therefore its effectiveness, may be influenced by the genomic location of the transgene insertion. Specifically, the effectiveness of the event can be impacted by cis and/or trans factors relative to the integration site or chromatin structure. Events can have the same transgenic insertion and nonetheless have different transgene expression levels and performance across tissues and developmental stages, in various germplasm, or under specific growth conditions. There may also be undesirable phenotypic or agronomic differences between some events. Therefore, it is necessary to produce and analyze a large number of individual plant transformation events in order to select an event having superior properties relative to the desirable trait, and the optimal phenotypic and agricultural characteristics necessary to make it suitable for commercial purposes. Further, the creation of a multi-gene event for commercial use requires rigorous molecular characterization, greenhouse testing, and field trials over multiple years, in multiple locations, and under a variety of conditions so extensive agronomic, phenotypic, and molecular data may be collected. The resulting data must then be analyzed by scientists and agronomists to select an event that is useful for commercial purposes. Once selected, such an event may then be introgressed using plant breeding methods as a single locus having multiple insect resistance traits into new germplasm suitably adapted to specific local growing conditions, and stacked/combined by breeding with other different events conferring traits different from the traits conferred by the event of the present invention.

### SUMMARY OF THE INVENTION

The invention provides a novel transgenic corn event—MON 95379—that provides insecticidal control over Lepidopteran pests of corn. The invention also provides transgenic plants, plant cells, seeds, plant parts, and commodity products comprising event MON 95379. In another embodiment, the invention provides polynucleotides specific for event MON 95379 and plants, plant cells, seeds, plant parts, progeny plants, and commodity products comprising event MON 95379. In yet another embodiment, methods related to event MON 95379 are provided.

Thus, in one aspect the invention provides a recombinant DNA molecule comprising a nucleotide sequence selected from the group consisting of SEQ ID NO:1, SEQ ID NO:2. SEQ ID NO:3. SEQ ID NO:4. SEQ ID NO:5. SEQ ID NO:6, SEQ ID NO:7, SEQ ID NO:8, SEQ ID NO:9, SEQ ID NO:10, and a complete complement thereof.

In one embodiment, the recombinant DNA molecule is derived from corn event MON 95379 in a sample of seed containing said event having been deposited as ATCC Accession No. PTA-125027.

Another aspect of the invention provides a DNA molecule comprising a polynucleotide segment of sufficient length to function as a DNA probe that hybridizes specifically under stringent hybridization conditions with corn event MON 95379 DNA in a sample, wherein detecting hybridization of said DNA molecule under said stringent hybridization conditions is diagnostic for the presence of corn event MON 95379 DNA in said sample. In certain embodiments, the sample comprises a corn plant, corn plant cell, corn seed, corn plant part, corn progeny plant, processed corn seed, animal feed comprising corn, corn oil, corn meal, corn flour, corn flakes, corn bran, pasta made with corn, corn biomass, and fuel products produced using corn and corn parts.

Yet another aspect of the invention provides a pair of DNA molecules, comprising a first DNA molecule and a second DNA molecule different from the first DNA molecule, that function as DNA primers when used together in an amplification reaction with a sample containing corn event MON 95379 template DNA to produce an amplicon diagnostic for the presence of said corn event MON 95379 DNA in said sample, wherein said amplicon comprises the nucleotide sequence selected from the group consisting of SEQ ID NO:1. SEQ ID NO:2. SEQ ID NO:3. SEQ ID NO:4. SEQ ID NO:5. SEQ ID NO:6, SEQ ID NO:7. SEQ ID NO:8, SEQ ID NO:9, and SEQ ID NO:10.

Another embodiment of the invention is a method of detecting the presence of a DNA segment diagnostic for corn event MON 95379 DNA in a sample, said method comprising: a) contacting the sample with a DNA molecule that functions as a probe and hybridizes specifically under stringent conditions with corn event MON 95379; b) subjecting said sample and said DNA molecule to stringent hybridization conditions; and c) detecting hybridization of said DNA molecule to said DNA in said sample, wherein said detection is diagnostic for the presence of said corn event MON 95379 DNA in said sample.

Yet another embodiment of the invention is a method of detecting the presence of a DNA segment diagnostic for corn event MON 95379 DNA in a sample, the method comprising: a) contacting said sample with the pair of DNA molecules of the invention; b) performing an amplification reaction sufficient to produce a DNA amplicon; and c) detecting the presence of said DNA amplicon in said reaction, wherein said DNA amplicon comprises the nucleotide sequence selected from the group consisting of SEQ ID NO:1. SEQ ID NO:2. SEQ ID NO:3. SEQ ID NO:4. SEQ ID NO:5, SEQ ID NO:6, SEQ ID NO:7, SEQ ID NO:8, SEQ ID NO:9, and SEQ ID NO:10.

Another embodiment of the invention is a corn plant, corn plant part, corn cell, or part thereof comprising a recombinant polynucleotide molecule comprising the nucleotide sequence selected from the group consisting of SEQ ID NO:1. SEQ ID NO:2. SEQ ID NO:3. SEQ ID NO:4. SEQ ID NO:5. SEQ ID NO:6, SEQ ID NO:7, SEQ ID NO:8. SEQ ID NO:9, and SEQ ID NO:10. This corn plant, corn plant part, corn cell, or part thereof is insecticidal when provided in the diet of a Lepidopteran insect pest. Lepidopteran pests can include Fall Armyworm (Spodoptera frugiperda). Corn Earworm (Helicoverpa zea), Southwestern Corn Borer (Diatraea grandiosella). Surgarcane Borer (Diatraea saccharalis), and Lesser Cornstalk Borer (Elasmopalpus lignosellus). In addition, the corn plant can be further defined as progeny of any generation of a corn plant comprising the corn event MON 95379.

Yet another embodiment of the invention is a method for protecting a corn plant from insect infestation, wherein said method comprises providing in the diet of a Lepidopteran insect pest an insecticidally effective amount of cells or tissue of a corn plant comprising corn event MON 95379. Again, contemplated Lepidopteran insect pests include Fall Armyworm (Spodoptera frugiperda), Corn Earworm (Helicoverpa zea), Southwestern Corn Borer (Diatraea grandiosella), Surgarcane Borer (Diatraea saccharalis), and Lesser Cornstalk Borer (Elasmopalpus lignosellus).

Another embodiment of the invention is a method of producing an insect resistant corn plant comprising: a) sexually crossing two different corn plants with at least one of the two different corn plants comprising transgenic corn event MON 95379 DNA; b) sampling seed or tissue from the progeny of step (a); c) detecting in said sample from step (b) progeny comprising corn event MON 95379 DNA; and d) selecting said progeny comprising corn event MON 95379 DNA.

A further embodiment of the invention is a corn seed, nonliving corn plant material, or a microorganism comprising a detectable amount of the nucleotide sequence selected from the group consisting of SEQ ID NO:1. SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, or complete complements thereof.

Yet another embodiment of the invention is a commodity product comprising a nucleotide sequence selected from the group consisting of SEQ ID NO:1, SEQ ID NO:2, SEQ ID NO:3, SEQ ID NO:4, SEQ ID NO:5. SEQ ID NO:6, SEQ ID NO:7. SEQ ID NO:8, SEQ ID NO:9, and SEQ ID NO:10, or complete complements thereof. Contemplated commodity products include whole or processed corn seed, animal feed comprising corn, corn oil, corn meal, corn flour, corn flakes, corn bran, corn biomass, and fuel products produced using corn and corn parts.

Another embodiment of the invention is a corn plant, corn plant part, or corn seed thereof comprising DNA functional as a template when tested in DNA amplification method producing an amplicon diagnostic for the presence of event MON 95379 DNA.

Yet another embodiment of the invention is a method of determining the zygosity of a corn plant or corn seed comprising event MON 95379 comprising: a) contacting a sample comprising corn DNA with a primer pair that is capable of producing an amplicon of one of the toxin coding sequences encoding Cry1B.868 or Cry1Da_7; b) contacting said sample comprising corn DNA with a primer pair that is capable of producing an amplicon of an internal standard known to be single-copy and homozygous in the corn plant; c) contacting the DNA sample with a probe set which contains at least a first probe that specifically hybridizes to one of the toxin coding sequences encoding Cry1B.868 or Cry1Da_7, and a second probe that specifically hybridizes to the internal standard genomic DNA known to be single-copy and homozygous in the corn plant; d) performing a DNA amplification reaction using real-time PCR and determining the cycle thresholds (Ct values) of the amplicon corresponding to the toxin coding sequence and the single-copy, homozygous internal standard; e) calculating the difference (ΔCt) between the Ct value of the single-copy, homozygous internal standard amplicon and the Ct value of the toxin coding sequence amplicon; and f) determining zygosity, wherein a ΔCt of about zero (0) indicates homozygosity of the inserted T-DNA of event MON 95739 and a ΔCt of about one (1) indicates heterozygosity of the inserted T-DNA of event MON 95379. In certain embodiments of this method, the primer pairs are selected from the group consisting of SEQ ID NO:18 combined with SEQ ID NO:19, and SEQ ID NO:21 combined with SEQ ID NO:22; and the probes are SEQ ID NO:20 and SEQ ID NO:23. In another embodiment, the primer pairs are selected from the group consisting of SEQ ID NO:18 combined with SEQ ID NO:19, and SEQ ID NO:24 combined with SEQ ID NO:25; and the probes are SEQ ID NO:20 and SEQ ID NO:26. In yet another embodiment of this invention the ΔCt of about one (1) indicating heterozygosity of the inserted T-DNA of event MON 95379 is in the range of 0.75 to 1.25.

A further embodiment of the invention is a method of determining the zygosity of a corn plant or corn seed comprising event MON 95379 comprising: a) contacting a sample comprising corn DNA with a set of primer pairs comprising at least two different primer pairs capable of producing a first amplicon diagnostic for event MON 95379 and a second amplicon diagnostic for native corn genomic DNA not comprising event MON 95379; i) performing a nucleic acid amplification reaction with the sample and the set of primer pairs; ii) detecting in the nucleic acid amplification reaction the first amplicon diagnostic for event MON 95379, or the second amplicon diagnostic for native corn genomic DNA not comprising event MON 95379, wherein the presence of only the first amplicon is diagnostic of a corn plant or corn seed homozygous for event MON 95379, and the presence of both the first amplicon and the second amplicon is diagnostic of a corn plant or corn seed heterozygous for event MON 95379; or b) contacting a sample comprising corn DNA with a probe set which contains at least a first probe that specifically hybridizes to event MON 95379 DNA and at least a second probe that specifically hybridizes to corn genomic DNA that was disrupted by insertion of the heterologous DNA of event MON 95379 and does not hybridize to event MON 95379 DNA; i) hybridizing the probe set with the sample under stringent hybridization conditions, wherein detecting hybridization of only the first probe under the hybridization conditions is diagnostic for a corn plant or corn seed homozygous for event MON 95379, and wherein detecting hybridization of both the first probe and the second probe under the hybridization conditions is diagnostic for a corn plant or a corn seed heterozygous for event MON 95379. In one embodiment of this method, the set of primer pairs comprises SEQ ID NO:15 combined with SEQ ID NO:16, and SEQ ID NO:15 combined with SEQ ID NO:27. In another embodiment of this method, the probe set comprises SEQ ID NO:17 and SEQ ID NO:28.

The forgoing and other aspects of the invention will become more apparent from the following detailed description.

### BRIEF DESCRIPTION OF THE DRAWINGS

FIG. 1 represents the sequence of corn event MON 95379. Horizontal lines and boxes correspond to the positions of SEQ ID NO:1 ([1]), SEQ ID NO:2 ([2]), SEQ ID NO:3 ([31), SEQ ID NO:4 ([4]), SEQ ID NO:5 ([5]), SEQ ID NO:6 ([6]). SEQ ID NO:7 ([7]). SEQ ID NO:8 ([8]), SEQ ID NO:9 ([9]). SEQ ID NO:1 (11 [11), and SEQ ID NO:12 ([12]) relative to SEQ ID NO:10 ([10]). The horizontal arrows labeled SQ51219 (SEQ ID NO:15) ([15]). SQ21524 (SEQ ID NO:16) ([16]), SQ50998 (SEQ ID NO:21) ([21]). SQ50997 (SEQ ID NO:22) ([22]), SQ50485 (SEQ ID NO:24) (124]) and SQ50484 (SEQ ID NO:25) ([25]) represent the approximate position of subsets of primers that can be used to detect corn event MON 95379. The horizontal arrows labeled PB10269 (SEQ ID NO:17) (117]), PB50340 (SEQ ID NO:23) ([23]). PB50138 (SEQ ID NO:26) ([26]) represent the approximate position of a DNA probe that can be used to detect corn event MON 95379. “E” represents an enhancer element, “P” represents a promoter element, “L” represents a leader (5′ UTR) element, “I” represents an intron element, “T” represents a 3′ UTR, “Cry1B.868” represents the Cry1B.868 coding sequence element, “Cry1Da_7” represents the Cry1 Da_7 coding sequence element. “LoxP” represents the site at which Cre-recombinase marker excision occurred, leaving behind one of the two LoxP sites after marker excision, and “LB” represents the left T-DNA border.

FIG. 2 is a diagrammatic representation of the T-DNA cassettes before integration to form event MON 95379, after integration, and after Cre-excision. The top horizontal box represents the T-DNA cassette in the plasmid vector used to transform event MON 95379, presented as SEQ ID NO:13 ([13]) (“T-DNA Before Integration”). The horizontal arrows below [13] represent the individual genetic elements comprised within the two transgene cassettes. “LB” represents a T-DNA left border element, “E” represents an enhancer element, “P” represents a promoter element, “L” represents a leader (5′ UTR) element, “I” represents an intron element, “T” represents a 3′ UTR, “Cry1B.868” represents the Cry1B.868 coding sequence element, “Cry1Da_7” represents the Cry1Da_7 coding sequence element, “CP4” represents the CP4 selectable marker, “TS” represented a targeting sequence, “LoxP” represents the site at which Cre-recombinase marker excision occurs, and “RB” represents a T-DNA right border element. The middle horizontal box, “Inserted T-DNA After Integration,” represents the T-DNA cassette integrated into the corn genome after transformation wherein the right T-DNA border (RB) was lost during integration. The bottom horizontal box. “Inserted T-DNA After Cre-Excision,” represents the integrated T-DNA cassette after the CP4 selectable marker cassette was excised, leaving behind one of the two LoxP sites and the LB region.

FIG. 3 is a diagrammatic representation of the breeding process to produce the marker-free corn event MON 95379. R₀ generation events (“transformants”) are those that are derived from the initial transformation with the binary transformation vector used to generate corn event MON 95379. Subsequent “R” generations (R₁, and R₂) represent successive generations produced through self-pollination of plants derived from the initial R₀ transformant that resulted in the corn event MON 95379. The R₂ transformants which are homozygous for the T-DNA insertion are cross-pollinated with an elite transgenic corn line comprising a transgene cassette for the expression of Cre-recombinase, resulting in an F1 generation, wherein many of the progeny have lost the CP4 selectable marker cassette due to Cre-recombinase excision. Hemizygous T-DNA positive. CP4 negative plants are selected and self-pollinated, resulting in an F2 generation. F2 plants homozygous for the inserted T-DNA allele without the CP4 marker and lacking the Cre-recombinase transgene cassette are selected and self-pollinated giving rise to an F3 generation. The F₃ generation plants are self-pollinated giving rise to a pure line of F₄ Gold Standard Seed.

### BRIEF DESCRIPTION OF THE SEQUENCES

SEQ ID NO:1 is a 50 nucleotide sequence representing the 5′ junction region of corn genomic DNA and the integrated transgenic expression cassette. SEQ ID NO:1 is found within SEQ ID NO:10 at nucleotide positions 838-887.

SEQ ID NO:2 is a 50 nucleotide sequence representing the 3′ junction region of the integrated transgenic expression cassette and the corn genomic DNA. SEQ ID NO:2 is found within SEQ ID NO:10 at nucleotide positions 14,156-14,205.

SEQ ID NO:3 is a 100 nucleotide sequence representing the 5′ junction region of corn genomic DNA and the integrated transgenic expression cassette. SEQ ID NO:3 is found within SEQ ID NO:10 at nucleotide positions 813-912.

SEQ ID NO:4 is a 100 nucleotide sequence representing the 3′ junction region of the integrated transgenic expression cassette and the corn genomic DNA. SEQ ID NO:4 is found within SEQ ID NO:10 at nucleotide positions 14.131-14.230.

SEQ ID NO:5 is a 200 nucleotide sequence representing the 5′ junction region of corn genomic DNA and the integrated transgenic expression cassette. SEQ ID NO:5 is found within SEQ ID NO:10 at nucleotide positions 763-962.

SEQ ID NO:6 is a 200 nucleotide sequence representing the 3′ junction region of the integrated transgenic expression cassette and the corn genomic DNA. SEQ ID NO:6 is found within SEQ ID NO:10 at nucleotide positions 14,081-14.280.

SEQ ID NO:7 is a 1.160 nucleotide sequence representing the 5′ junction region of corn genomic DNA and the integrated transgenic expression cassette. SEQ ID NO:7 is found within SEQ ID NO:10 at nucleotide positions 1-1,160.

SEQ ID NO:8 is a 1,178 nucleotide sequence representing the 3′ junction region of the integrated transgenic expression cassette and the corn genomic DNA. SEQ ID NO:8 is found within SEQ ID NO:10 at nucleotide positions 14,039-15,216.

SEQ ID NO:9 is a 13.318 nucleotide sequence corresponding to the transgenic inserted T-DNA of corn event MON 95379.

SEQ ID NO:10 is a 15.216 nucleotide sequence corresponding to the contig nucleotide sequence of the 5′ genomic flanking DNA nucleotide sequence, the inserted T-DNA nucleotide sequence in event MON 95379, and the 3′ genomic flanking DNA nucleotide sequence; and includes SEQ ID NO:11 (nucleotides 1-862). SEQ ID NO:9 (nucleotides 863-14.180), and SEQ ID NO:12 (nucleotides 14.181-15,216).

SEQ ID NO:11 is an 862 nucleotide sequence representing the 5′ flanking corn genomic DNA up to the inserted T-DNA. SEQ ID NO:11 is found within SEQ ID NO:10 at nucleotide positions 1-862.

SEQ ID NO:12 is a 1.036 nucleotide sequence representing the 3′ flanking corn genomic DNA after the inserted T-DNA. SEQ ID NO:12 is found within SEQ ID NO:10 at nucleotide positions 14.181-15,216.

SEQ ID NO:13 is a 18,376 nucleotide sequence representing the transgene cassette comprised within the binary plasmid transformation vector used to transform corn to produce corn event MON 95379.

SEQ ID NO:14 is a 35 nucleotide sequence representing the LoxP sites used for Cre-mediated excision and recombination. A remaining LoxP site after marker excision can be found within SEQ ID NO:10 at nucleotide positions 1.080-1.114.

SEQ ID NO:15 is a 20 nucleotide sequence corresponding to a thermal amplification primer referred to as SQ51219 used to identify corn event MON 95379 DNA in a sample, and is identical to the nucleotide sequence corresponding to positions 833-852 of SEQ ID NO:10.

SEQ ID NO:16 is a 30 nucleotide sequence corresponding to a thermal amplification primer referred to as SQ21524 used to identify corn event MON 95379 DNA in a sample, and is identical to the reverse complement of the nucleotide sequence corresponding to positions 905-934 of SEQ ID NO:10.

SEQ ID NO:17 is a 16 nucleotide sequence corresponding to a probe referred to as PB10269 used to identify corn event MON 95379 DNA in a sample, and is identical to the reverse complement of the nucleotide sequence corresponding to positions 886-901 of SEQ ID NO:10.

SEQ ID NO:18 is a 24 nucleotide sequence corresponding to a thermal amplification primer referred to as SQ20222 used as an internal control for the event and zygosity assay for MON 95379 and hybridizes to a region of the corn genome.

SEQ ID NO:19 is a 28 nucleotide sequence corresponding to a thermal amplification primer referred to as SQ20221 used as an internal control for the event and zygosity assay for MON 95379 and hybridizes to a region of the corn genome.

SEQ ID NO:20 is a 29 nucleotide sequence corresponding to a probe referred to as PB50237 used as an internal control for the event and zygosity assay for MON 95379 and hybridizes to a region of the corn genome.

SEQ ID NO:21 is a 20 nucleotide sequence corresponding to a thermal amplification primer referred to as SQ50998 used in the zygosity assay for event MON 95379 and hybridizes to the coding sequence of Cry1B.868 within SEQ ID NO:10; and is identical to the nucleotide sequence corresponding to positions 2,809-2,828 of SEQ ID NO:10.

SEQ ID NO:22 is a 20 nucleotide sequence corresponding to a thermal amplification primer referred to as SQ50997 used in the zygosity assay for event MON 95379 and hybridizes to the coding sequence of Cry1B.868 within SEQ ID NO:10; and is identical to the reverse complement of the nucleotide sequence corresponding to positions 2,852-2,871 of SEQ ID NO:10.

SEQ ID NO:23 is an 18 nucleotide sequence corresponding to a probe referred to as PB50340 used in the zygosity assay for event MON 95379 and hybridizes to the coding sequence of Cry1B.868 within SEQ ID NO:10; and is identical to the reverse complement of the nucleotide sequence corresponding to positions 2,833-2,850 of SEQ ID NO:10.

SEQ ID NO:24 is a 19 nucleotide sequence corresponding to a thermal amplification primer referred to as SQ50485 used in the zygosity assay for event MON 95379 and hybridizes to the coding sequence of Cry1Da_7 within SEQ ID NO:10; and is identical to the nucleotide sequence corresponding to positions 12,820-12.838 of SEQ ID NO:10.

SEQ ID NO:25 is an 18 nucleotide sequence corresponding to a thermal amplification primer referred to as SQ50484 used in the zygosity assay for event MON 95379 and hybridizes to the coding sequence of Cry1Da_7 within SEQ ID NO:10; and is identical to the reverse complement of the nucleotide sequence corresponding to positions 12,855-12.872 of SEQ ID NO:10.

SEQ ID NO:26 is a 14 nucleotide sequence corresponding to a probe referred to as PB50138 used in the zygosity assay for event MON 95379 and hybridizes to the coding sequence of Cry1Da_7 within SEQ ID NO:10; and is identical to the reverse complement of the nucleotide sequence corresponding to positions 12,840-12,853 of SEQ ID NO:10.

SEQ ID NO:27 is a 21 nucleotide sequence corresponding to a thermal amplification primer referred to as PNEGDNA used in the zygosity assay for event MON 95379 and hybridizes to a region of corn genomic DNA which was deleted when the T-DNA used to produce event MON 95379 inserted into the corn genome. An amplicon derived from the combination of primers SQ51219 and PNEGDNA is diagnostic for the wild-type allele lacking the event MON 95379 inserted T-DNA.

SEQ ID NO:28 is a 14 nucleotide sequence corresponding to a probe referred to as PRBNEGDNA used in the zygosity assay for event MON 95379 and hybridizes to a region of corn genomic DNA which was deleted when the T-DNA used to produce event MON 95379 inserted into the corn genome.

### DETAILED DESCRIPTION

The present invention provides a transgenic corn event—MON 95379—that achieves insecticidal control over Lepidopteran pests of corn by expression of Cry1B.868 and Cry1 Da_7. Specifically, expression of the Cry1B.868 and Cry1Da_7 insect inhibitory proteins in corn event MON 95379 provides resistance to the Lepidopteran insect pests Fall Armyworm (Spodoptera frugiperda), Corn Earworm (Helicoverpa zea), Southwestern Corn Borer (Diatraea grandiosella), Surgarcane Borer (Diatraea saccharalis), and Lesser Cornstalk Borer (Elasmopalpus lignosellus). Event MON 95379 will meet a great need for control of these insects in the corn market, as chemical insecticides often do not provide adequate control of these insects, or require multiple applications over the growing season, increasing the input of chemical pesticides in the environment and adding cost to the production of corn.

It should be understood that reference to event MON 95379 is equivalent to reference to event MON95379; they are interchangeable and represent the same transgenic corn event.

Plant transformation techniques are used to insert foreign DNA (also known as transgenic DNA) randomly into a chromosome of the genome of a cell to produce a genetically engineered cell, also referred to as a “transgenic” or “recombinant” cell. Using this technique, many individual cells are transformed, each resulting in a unique “transgenic event” or “event” due to the random insertion of the foreign DNA into the genome. A transgenic plant is then regenerated from each individual transgenic cell. This results in every cell of the transgenic plant containing the uniquely inserted transgenic event as a stable part of its genome. This transgenic plant can then be used to produce progeny plants, each containing the unique transgenic event.

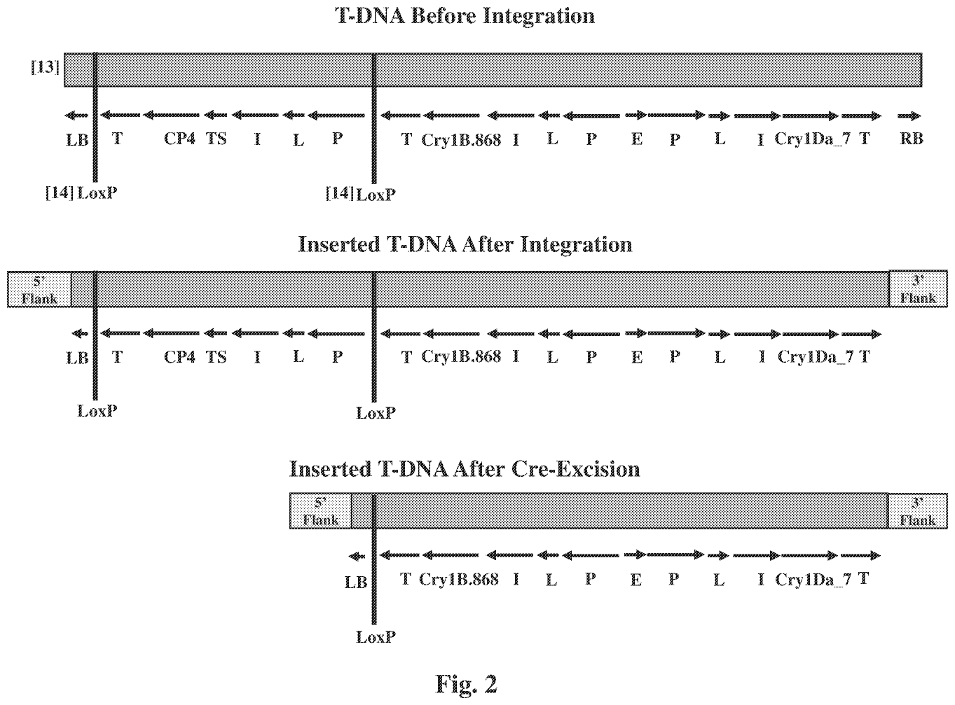

Corn event MON 95379 was produced by an Agrobacterium-mediated transformation process of corn immature embryos with a single T-DNA binary system. In this system, an Agrobacterium strain employing one binary plasmid vector with a single T-DNA was utilized. The T-DNA construct comprised two transgene cassettes for the expression of the insect toxin coding sequences encoding Cry1B.868 and Cry1Da_7, and a transgene cassette used for the selection of transformed corn cells using glyphosate selection (CP4). The T-DNA construct is SEQ ID NO:13, and illustrated in FIG. 2 (“T-DNA Before Integration”). During integration, the right T-DNA border was lost as shown in FIG. 2 (“Inserted T-DNA After Integration”). The glyphosate selection cassette was flanked on both sides with LoxP recognition sites which are recognized by Cre-recombinase, derived from Enterobacteria phage P1 (Larry Gilbertson (2003) Cre-lox recombination: Cre-active tools for plant biotechnology. TRENDS in Biotechnology. 21:12, 550-555).

As specifically described herein, corn event MON 95379 was produced by a complex research and development process in which: (1) hundreds of plasmid vector constructs—which varied with respect to the coding sequences for the insecticidal proteins, the coding sequences for the transcriptional regulatory elements, and number and orientation of the cassettes within the constructs—were developed and transformed into corn cells to create thousands of events that were tested and analyzed, resulting in the selection of the construct used to generate event MON 95379; (2) hundreds of corn cells were transformed with the construct used to generate event MON 95379, creating a population of transgenic plants in which each plant contained a unique transgenic event that was regenerated and tested; (3) the final event MON 95379 was selected after a rigorous multi-year event selection process involving the testing and analysis of molecular characteristics, efficacy, protein expression, and agronomic properties in a variety of genetic backgrounds; and (4) the glyphosate selection cassette in corn event MON 95379 was removed through in vivo Cre-excision to create a “marker-free” final event MON 95379. Corn event MON 95379 was thus produced and selected as a uniquely superior event useful for broad-scale agronomic purposes.

The plasmid DNA inserted into the genome of corn event MON 95379 was characterized by detailed molecular analysis. This analysis included: the insert number (number of integration sites within the corn genome), the genomic insert location (the specific site in the corn genome where the insertion occurred), the copy number (the number of copies of the T-DNA within one locus), and the integrity of the transgenic inserted DNA. The detailed molecular analysis demonstrated that the integrated T-DNA containing the Cry1B.868 and Cry1Da_7 expression cassettes remained intact after integration and Cre-excision of the glyphosate (CP4) selection cassette. As used herein, an “expression cassette” or “cassette” is a recombinant DNA molecule comprising a combination of distinct elements that are to be expressed by a transformed cell. Table 1 provides a list of the elements contained in SEQ ID NO:10, the DNA sequence that corresponds to corn event MON 95379.

TABLE 1
Description of corn event MON 95379

| | Position in | |
|---|---|---|
| | SEQ ID | |
| Element | NO: 10 | Description |
| 5′ Flanking DNA | 1-862 | DNA sequence flanking the 5′ end of |
| | | the transgenic insert. |
| Left Border Region | 863-1044 | DNA region from Agrobacterium |
| | | tumefaciens containing the left border |
| | | sequence. |
| LoxP | 1080-1114 | A recognition sequence for a site- |
| | | specific recombinase from |
| | | Enterobacteria phage P1. |
| T-Os.LTP:1 | 1225-1524 | The 3′ untranslated region for a Lipid |
| | | Transfer Protein-like gene (LTP) from |
| | | Oryza sativa (rice). |
| Cry1B.868 | 1534-5133 | Coding sequence of a chimeric insect |
| | | toxin comprised of domains 1 and 2 of |
| | | Cry1Be2, domain 3 of Cry1Ca, and |
| | | the protoxin domain of Cry1Ab3. |
| I-Zm.UbqM1-1:1:16 | 5160-6212 | An intron derived from an Ubquitin 1 |
| | | gene of Zea mays Mexicana. |
| L-Zm.UbqM1-1:1:4 | 6213-6290 | A 5′ UTR derived from an Ubquitin 1 |
| | | gene of Zea mays Mexicana. |
| P-Zm.UbqM1-1:1:5 | 6291-7167 | A promoter derived from an Ubquitin |
| | | 1 gene of Zea mays Mexicana. |
| E-FMV.35S-1:1:2 | 7195-7731 | The enhancer of the 35S gene from |
| | | Figwort Mosaic Virus (FMV). |
| P-SETit.Tip-1:1:1 | 7743-8659 | A promoter of a tonoplast membrane |
| | | integral protein gene from Setaria |
| | | italica (foxtail millet). |
| L-SETit.Tip-1:1:1 | 8660-8723 | A 5′ UTR of a tonoplast membrane |
| | | integral protein gene from Setaria |
| | | italica (foxtail millet). |
| I-Os.Act15-1:1:1 | 8732-10024 | The first intron from the Actin 15 (Act |
| | | 15) gene from Oryza sativa (rice). |
| Cry1Da_7 | 10043-13543 | Coding sequence of a Cry1Da insect |
| | | toxin with amino acid modifications to |
| | | improve efficacy. |
| T-Os.GOS2-1:1:1 | 13560-14027 | The 3′ untranslated region from the |
| | | GOS2 gene encoding a translation |
| | | initiation factor from Oryza sativa |
| | | (rice). |
| 3′ Flanking Sequence | 14181-15216 | DNA sequence flanking the 3′ end of |
| | | the transgenic insert. |

Corn event MON 95379 is characterized as an insertion into a single locus in the corn genome, resulting in two new loci or junction sequences (e.g., sequences set forth in SEQ ID NO:1, SEQ ID NO:2, SEQ ID NO:3. SEQ ID NO:4. SEQ ID NO:5. SEQ ID NO:6, SEQ ID NO:7, and SEQ ID NO:8) spanning a portion of the inserted DNA and the corn genomic DNA that are not known to appear naturally in the corn genome or other transgenic corn events—they are unique to event MON 95379. These junction sequences are useful in detecting the presence of the event MON 95379 in corn cells, corn tissue, corn seed, and corn plants or corn plant products, such as corn commodity products. DNA molecular probes and primer pairs are described herein that have been developed for use in identifying the presence of these various junction segments in biological samples containing or suspected of containing corn cells, corn seed, corn plant parts, or corn plant tissue that contain the event MON 95379.

A sample is intended to refer to a composition that is either substantially pure corn DNA or a composition that contains corn DNA. In either case, the sample is a biological sample. i.e., it contains biological materials, including but not limited to DNA obtained or derived from, either directly or indirectly, the genome of corn event MON 95379. “Directly” refers to the ability of the skilled artisan to directly obtain DNA from the corn genome by fracturing corn cells (or by obtaining samples of corn that contain fractured corn cells) and exposing the genomic DNA for the purposes of detection. “Indirectly” refers to the ability of the skilled artisan to obtain the target or specific reference DNA, i.e., a novel and unique junction segment described herein as being diagnostic for the presence of the event MON 95379 in a particular sample, by means other than by obtaining directly via fracturing of corn cells or obtaining a sample of corn that contains fractured corn cells. Such indirect means include, but are not limited to, amplification of a DNA segment that contains the DNA sequence targeted by a particular probe designed to bind with specificity to the target sequence, or amplification of a DNA segment that can be measured and characterized, i.e., measured by separation from other segments of DNA through some efficient matrix such as an agarose or acrylamide gel or the like, or characterized by direct sequence analysis of the amplicons, or cloning of the amplicon into a vector and direct sequencing of the inserted amplicon present within such vector.

Detailed molecular analysis demonstrated that event MON 95379 contains a single T-DNA insertion with one copy of each of the Cry1B.868 and Cry1Da_7 expression cassettes. No additional elements from the transformation construct other than portions of the Agrobacterium tumefaciens left border region used for transgenic DNA transfer from the plant transformation plasmid to the corn genome were identified in event MON 95379. Finally, thermal amplification producing specific amplicons diagnostic for the presence of event MON 95379 in a sample and DNA sequence analyses were performed to determine the arbitrarily assigned 5′ and 3′ insert-to-plant genome junctions, confirm the organization of the elements within the insert, and determine the complete DNA sequence of the inserted transgenic DNA (SEQ ID NO:9). SEQ ID NO:11 is a sequence representing the eight hundred sixty-two (862) base-pair (bp) 5′ LH244 corn genomic DNA sequence flanking the inserted T-DNA sequence presented as SEQ ID NO:9. SEQ ID NO:12 is a sequence representing the one thousand thirty-six (1.036) bp 3′ LH244 corn genomic DNA sequence flanking the inserted T-DNA sequence presented as SEQ ID NO:9. SEQ ID NO:7 is a sequence representing the eight hundred sixty-two (862) base-pair (bp) 5′ LH244 corn genomic DNA sequence flanking the inserted T-DNA sequence combined with two hundred ninety-eight (298) bp of inserted T-DNA sequence presented as SEQ ID NO:9. SEQ ID NO:8 is a sequence representing one hundred forty-two (142) bp of inserted T-DNA sequence with the one thousand thirty-six (1.036) bp 3′ LH244 corn genomic DNA sequence flanking the inserted T-DNA sequence presented as SEQ ID NO:9. SEQ ID NO:10 corresponds to corn event MON 95379 and contains a contiguous sequence (contig) comprising the 5′ LH244 flanking sequence, the transgene insert of event MON 95379, and the 3′ LH244 flanking sequence, and thus contains the insert-to-plant genome junction sequences.

Unless otherwise noted herein, terms are to be understood according to conventional usage by those of ordinary skill in the relevant art. Definitions of common terms in molecular biology may be found in Rieger et al., Glossary of Genetics: Classical and Molecular, 5′ edition, Springer-Verlag: New York, 1991; and Lewin, Genes V, Oxford University Press: New York, 1994, along with other sources known to those of ordinary skill in the art. As used herein, the term “corn” means species belong to the genus Zea, preferably Zea nays and includes all plant varieties that can be bred with corn plants containing event MON 95379, including wild corn species as well as those plants belonging to the genus Zea that permit breeding between species.

The present invention provides for transgenic plants which have been transformed with a DNA construct that contains expression cassettes expressing toxic amounts of the insecticidal proteins Cry1B.868 and Cry1Da_7. What is meant by toxic amount is an efficacious amount, an insecticidal amount, an insecticidally-effective amount, a target insect suppressive amount, an efficacious pesticidal amount, an amount in the diet of insects in the order of Lepidoptera that is insecticidal, and other similar terms to be understood according to conventional usage by those of ordinary skill in the relevant art. Corn plants transformed according to the methods and with the DNA construct disclosed herein are resistant to Lepidopteran insect pests.

A transgenic “plant” is produced by transformation of a plant cell with heterologous DNA, i.e., a polynucleic acid construct that includes a number of efficacious features of interest, regeneration of a plant resulting from the insertion of the transgene into the genome of the plant cell, and selection of a particular plant characterized by insertion into a particular genome location and the number of efficacious features of the regenerated transgenic plant. The term “event” refers to DNA from the original transformant comprising the inserted DNA, and flanking genomic sequences immediately adjacent to the inserted DNA. Such DNA is unique and would be expected to be transferred to a progeny that receives the inserted DNA, including the transgene of interest, as the result of a sexual cross of a parental line that includes the inserted DNA (e.g., the original transformant and progeny resulting from selfing) and a parental line that does not contain the inserted DNA. The present invention also provides the original transformant plant and progeny of the transformant that include the heterologous DNA. Such progeny may be produced by a sexual outcross between plants comprising the event and another plant wherein the progeny includes the heterologous DNA. Even after repeated back-crossing to a recurrent parent, the event is present in the progeny of the cross at the same chromosomal location.

As used herein, the term “recombinant” refers to a non-natural DNA, protein, or organism that would not normally be found in nature and was created by human intervention. A “recombinant DNA molecule” is a DNA molecule comprising a combination of DNA molecules that would not naturally occur together and is the result of human intervention. For example, a DNA molecule that is comprised of a combination of at least two DNA molecules heterologous to each other, such as a DNA molecule that comprises a transgene and the plant genomic DNA adjacent to the transgene, is a recombinant DNA molecule.

The terms “DNA” and “DNA molecule” referred to herein refer to a deoxyribonucleic acid (DNA) molecule. A DNA molecule may be of genomic or synthetic origin, and is by convention from the 5′ (upstream) end to the 3′ (downstream) end. As used herein, the term “DNA sequence” refers to the nucleotide sequence of the DNA molecule. By convention, the DNA sequences of the invention and fragments thereof are disclosed with reference to only one strand of the two strand complementary DNA sequence strands. By implication and intent, the complementary sequences of the sequences provided here (the sequences of the complementary strand), also referred to in the art as the reverse complementary sequences, are within the scope of the invention and are expressly intended to be within the scope of the subject matter claimed.

As used herein, the term “fragment” refers to a smaller piece of the whole. For example, fragments of SEQ ID NO:10 would include sequences that are at least about 12 consecutive nucleotides, at least about 13 consecutive nucleotides, at least about 14 consecutive nucleotides, at least about 15 consecutive nucleotides, at least about 16 consecutive nucleotides, at least about 17 consecutive nucleotides, at least about 18 consecutive nucleotides, at least about 19 consecutive nucleotides, at least about 20 consecutive nucleotides, at least about 25 consecutive nucleotides, at least about 30 consecutive nucleotides, at least about 35 consecutive nucleotides, at least about 40 consecutive nucleotides, at least about 45 consecutive nucleotides, at least about 50 consecutive nucleotides, at least about 60 consecutive nucleotides, at least about 70 consecutive nucleotides, at least about 80 consecutive nucleotides, at least about 90 consecutive nucleotides, or at least about 100 consecutive nucleotides of the complete sequence of SEQ ID NO:10.

Reference in this application to an “isolated DNA molecule” or an equivalent term or phrase is intended to mean that the DNA molecule is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding an insecticidal protein or any naturally occurring insecticidal variant of that protein would be an isolated nucleotide sequence so long as the nucleotide sequence was not within the DNA of the bacterium from which the sequence encoding the protein is naturally found. A synthetic nucleotide sequence encoding the amino acid sequence of the naturally occurring insecticidal protein would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium. In any circumstance, the isolated DNA molecule is a chemical molecule, regardless of whether it is referred to as a nucleic acid, a nucleic acid sequence, a polynucleotide sequence, and the like. It is a novel, inventive molecule that exhibits industrial applicability both when present in a plant cell or in a plant genome, and when present outside of a plant cell, and therefore, exhibits and is intended to exhibit such utility regardless of where the molecule is located.

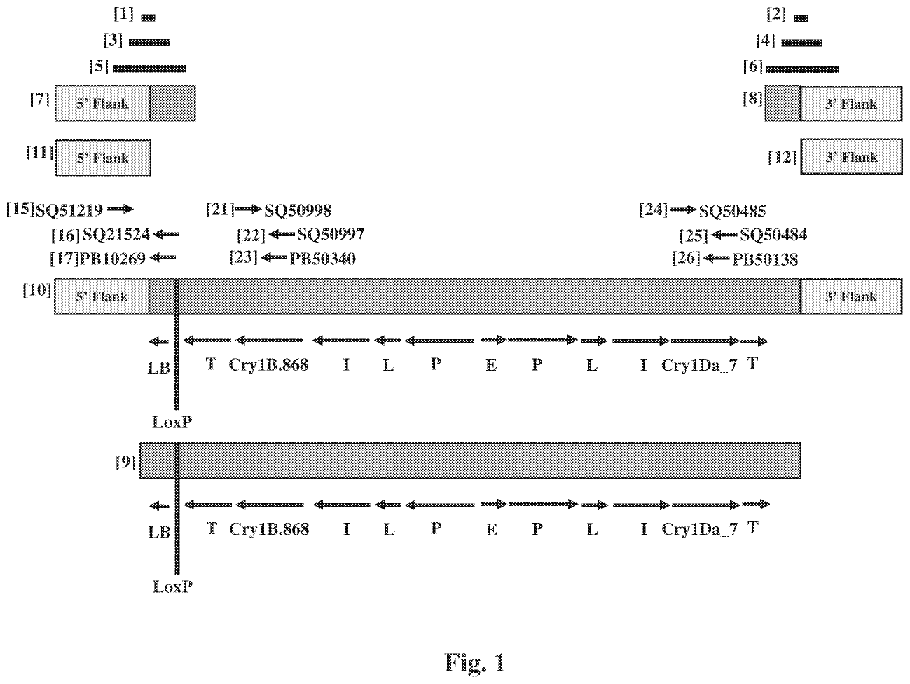

The DNA sequence of the region spanning the connection by phosphodiester bond linkage of one end of the transgenic insert to the flanking corn genomic DNA is referred to as a “junction.” A junction is the connection point of the transgenic insert and flanking DNA as one contiguous molecule. One junction is found at the 5′ end of the transgenic insert and the other is found at the 3′ end of the transgenic insert, referred to herein as the 5′ and 3′ junction, respectively. A “junction sequence” refers to a DNA sequence of any length that spans the 5′ or 3′ junction of an event. Junction sequences of corn event MON 95379 are apparent to one of skill in the art using SEQ ID NO:10. Examples of junction sequences of event MON 95379 are provided as SEQ ID NOs:1-8. FIG. 1 illustrates the physical arrangement of the junction sequences, arranged from 5′ to 3′, relative to SEQ ID NO:10. The junction sequences of event MON 95379 may be present as part of the genome of a plant, seed, or cell containing event MON 95379. The identification of any one or more of the junction sequences in a sample from a plant, plant part, seed, or cell indicates that the DNA was obtained from corn containing event MON 95379, and is diagnostic for the presence of event MON 95379.

The junction sequences for event MON 95379 may be represented by a sequence from the group consisting of SEQ ID NO:1. SEQ ID NO:2. SEQ ID NO:3, SEQ ID NO:4, SEQ ID NO:5. SEQ ID NO:6. SEQ ID NO:7, SEQ ID NO:8, and SEQ ID NO:10. For example, the junction sequences may be arbitrarily represented by the nucleotide sequences provided as SEQ ID NO:1 and SEQ ID NO:2. Alternatively, the junction sequences may be arbitrarily represented by the nucleotide sequences provided as SEQ ID NO:3 and SEQ ID NO:4. Alternatively, the junction sequences may be arbitrarily represented by the nucleotide sequences provided as SEQ ID NO:5 and SEQ ID NO:6. Alternatively, the junction sequences may be arbitrarily represented by the nucleotide sequences provided as SEQ ID NO:7 and SEQ ID NO:8. These nucleotides are connected by phosphodiester linkage, and in corn event MON 95379 are present as part of the recombinant plant cell genome.

The identification of one or more of SEQ ID NO:1. SEQ ID NO:2. SEQ ID NO:3, SEQ ID NO:4. SEQ ID NO:5, SEQ ID NO:6. SEQ ID NO:7, SEQ ID NO:8, and SEQ ID NO:10 in a sample derived from a corn plant, corn seed, or corn plant part is diagnostic that the DNA was obtained from corn event MON 95379. The invention thus provides a DNA molecule that contains at least one of the nucleotide sequences provided as SEQ ID NO:1, SEQ ID NO:2, SEQ ID NO:3. SEQ ID NO:4, SEQ ID NO:5, SEQ ID NO:6, SEQ ID NO:7. SEQ ID NO:8. SEQ ID NO:9, and SEQ ID NO:10. Any segment of DNA derived from transgenic corn event MON 95379 that is sufficient to include at least one of the sequences provided as SEQ ID NO:1. SEQ ID NO:2, SEQ ID NO:3. SEQ ID NO:4, SEQ ID NO:5, SEQ ID NO:6, SEQ ID NO:7, SEQ ID NO:8. SEQ ID NO:9, and SEQ ID NO:10 is within the scope of the invention. In addition, any polynucleotide comprising a sequence complementary to any of the sequences described within this paragraph is within the scope of the invention.

The invention provides exemplary DNA molecules that can be used either as primers or probes for detecting the presence of DNA derived from a corn plant comprising event MON 95379 DNA in a sample. Such primers or probes are specific for a target nucleic acid sequence and, as such, are useful for the identification of corn event MON 95379 nucleic acid sequence by the methods of the invention described herein.

A “probe” is a nucleic acid molecule that is complementary to a strand of target nucleic acid and is useful in hybridization methods. A probe may be attached a conventional detectable label or reporter molecule, e.g., a radioactive isotope, ligand, chemiluminescent agent, or enzyme. Such a probe is complementary to a strand of a target nucleic acid and, in the case of the present invention, to a strand of DNA from event MON 95379 whether from an event MON 95379 containing plant or from a sample that includes event MON 95379 DNA. Probes according to the present invention include not only deoxyribonucleic or ribonucleic acids, but also polyamides and other probe materials that bind specifically to a target DNA sequence and can be used to detect the presence of that target DNA sequence. Exemplary DNA sequences useful as a probe for detecting corn event MON 95379 are provided as: SEQ ID NO:17 (PB10269). SEQ ID NO:23 (PB50340); SEQ ID NO:26 (PB50138).

A “primer” is typically a DNA molecule that is designed for use in specific annealing or hybridization methods that involve thermal amplification. A pair of primers may be used with template DNA (such as a sample of corn genomic DNA) in a thermal amplification (such as polymerase chain reaction (PCR)) to produce an amplicon, where the amplicon produced from such reaction would have a DNA sequence corresponding to sequence of the template DNA located between the two sites where the primers hybridized to the template.

A primer is typically designed to hybridize to a complementary target DNA strand to form a hybrid between the primer and target DNA strand, and the presence of the primer is a point of recognition by a polymerase to begin extension of the primer (i.e., polymerization of additional nucleotides into a lengthening nucleotide molecule) using as a template the target DNA strand. Primer pairs refer to use of two primers binding opposite strands of a double stranded nucleotide segment for the purpose of amplifying linearly the polynucleotide segment between the positions targeted for binding by the individual members of the primer pair, typically in a thermal amplification reaction or other conventional nucleic-acid amplification methods. Exemplary DNA molecules useful as primers are provided as SEQ ID NO:15. SEQ ID NO:16. SEQ ID NO:18. SEQ ID NO:19, SEQ ID NO:21, SEQ ID NO:22, SEQ ID NO:24, SEQ ID NO:25 and SEQ ID NO:27.

The primer pair SEQ ID NO:15 and SEQ ID NO:16 are useful as a first DNA molecule and a second DNA molecule that is different from the first DNA molecule, and both are each of sufficient length of contiguous nucleotides of SEQ ID NO:10 to function as DNA primers that, when used together in a thermal amplification reaction with template DNA derived from corn event MON 95379, to produce an amplicon diagnostic for corn event MON 95379 DNA in a sample. The primer pair SEQ ID NO:21 and SEQ ID NO:22 are useful as a first DNA molecule and a second DNA molecule that is different from the first DNA molecule, and both are each of sufficient length of contiguous nucleotides of SEQ ID NO:10 to function as DNA primers that, when used together in a thermal amplification reaction with template DNA derived from corn event MON 95379, to produce an amplicon diagnostic for the zygosity of corn event MON 95379 DNA in a sample. The primer pair SEQ ID NO:24 and SEQ ID NO:25 are useful as a first DNA molecule and a second DNA molecule that is different from the first DNA molecule, and both are each of sufficient length of contiguous nucleotides of SEQ ID NO:10 to function as DNA primers that, when used together in a thermal amplification reaction with template DNA derived from corn event MON 95379, to produce an amplicon diagnostic for the zygosity of corn event MON 95379 DNA in a sample. The primer pair SEQ ID NO:18 and SEQ ID NO:19 are useful as a first DNA molecule and a second DNA molecule that is different from the first DNA molecule, and both are each of sufficient length of contiguous nucleotides of a locus within the corn genome to function as DNA primers that, when used together in a thermal amplification reaction with template DNA derived from corn event MON 95379, to produce an amplicon that serves as an internal control for both the diagnosis of corn event MON 95379, as well as the zygosity of corn event MON 95379 DNA in a sample.

DNA probes and DNA primers are generally eleven (11) polynucleotides or more in length, often eighteen (18) polynucleotides or more, twenty-four (24) polynucleotides or more, or thirty (30) polynucleotides or more. Such probes and primers are selected to be of sufficient length to hybridize specifically to a target sequence under high stringency hybridization conditions. Preferably, probes and primers according to the present invention have complete sequence similarity with the target sequence, although probes differing from the target sequence that retain the ability to hybridize to target sequences may be designed by conventional methods.

The nucleic acid probes and primers of the present invention hybridize under stringent conditions to a target DNA molecule. Any conventional nucleic acid hybridization or amplification method can be used to identify the presence of DNA from a transgenic plant in a sample. Polynucleic acid molecules also referred to as nucleic acid segments or fragments thereof are capable of specifically hybridizing to other nucleic acid molecules under certain circumstances.

As used herein, two polynucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., 1989, and by Haymes et al., In: Nucleic Acid Hybridization, A Practical Approach. IRL Press, Washington, DC (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. Appropriate stringency conditions that promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. In a preferred embodiment, a polynucleic acid of the present invention will specifically hybridize to one or more of the nucleic acid molecules set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10, or complements thereof or fragments thereof under moderately stringent conditions, for example at about 2.0×SSC and about 65° C. In a particularly preferred embodiment, a nucleic acid of the present invention will specifically hybridize to one or more of the nucleic acid molecules set forth in SEQ ID NO:1, SEQ ID NO:2. SEQ ID NO:3, SEQ ID NO:4, SEQ ID NO:5, SEQ ID NO:6. SEQ ID NO:7. SEQ ID NO:8, SEQ ID NO:9, or SEQ ID NO:10, or complements or fragments thereof under high stringency conditions. In one aspect of the present invention, a preferred marker nucleic acid molecule of the present invention has the nucleic acid sequence set forth in SEQ ID NO:1, or SEQ ID NO:2, or SEQ ID NO:3, or SEQ ID NO:4, or SEQ ID NO:5, or SEQ ID NO:6, or SEQ ID NO:7, or SEQ ID NO:8, or SEQ ID NO:9, or SEQ ID NO:10, or complements thereof, or fragments thereof. The hybridization of the probe to the target DNA molecule can be detected by any number of methods known to those skilled in the art, these can include, but are not limited to, fluorescent tags, radioactive tags, antibody based tags, and chemiluminescent tags.

Regarding the amplification of a target nucleic acid sequence (e.g., by PCR) using a particular amplification primer pair, “stringent conditions” are conditions that permit the primer pair to hybridize only to the target nucleic acid sequence to which a primer having the corresponding wild-type sequence (or its complement) would bind and preferably to produce a unique amplification product, the amplicon, in a DNA thermal amplification reaction.

The term “specific for (a target sequence)” indicates that a probe or primer hybridizes under stringent hybridization conditions only to the target sequence in a sample comprising the target sequence.

As used herein, “amplified DNA” or “amplicon” refers to the product of polynucleic acid amplification method directed to a target polynucleic acid molecule that is part of a polynucleic acid template. For example, to determine whether a corn plant resulting from a sexual cross contains transgenic plant genomic DNA from a corn plant comprising event MON 95379 of the present invention. DNA that is extracted from a corn plant tissue sample may be subjected to a polynucleic acid amplification method using a primer pair that includes a first primer derived from a genomic DNA sequence in the region flanking the heterologous inserted DNA of event MON 95379 and is elongated by polymerase 5′ to 3′ in the direction of the inserted DNA. The second primer is derived from the heterologous inserted DNA molecule is elongated by the polymerase 5′ to 3′ in the direction of the flanking genomic DNA from which the first primer is derived. The amplicon may range in length from the combined length of the primer pair plus one nucleotide base pair, or plus about fifty nucleotide base pairs, or plus about two hundred-fifty nucleotide base pairs, or plus about four hundred-fifty nucleotide base pairs or more. Alternatively, a primer pair can be derived from genomic sequence on both sides of the inserted heterologous DNA so as to produce an amplicon that includes the entire insert polynucleotide sequence (e.g., a forward primer isolated from the genomic portion on the 5′ end of SEQ ID NO:10 and a reverse primer isolated from the genomic portion on the 3′ end of SEQ ID NO:10 that amplifies a DNA molecule comprising the inserted DNA sequence (SEQ ID NO:9) identified herein in the event MON 95379 genome). A member of a primer pair derived from the plant genomic sequence adjacent to the inserted transgenic DNA is located a distance from the inserted DNA sequence, this distance can range from one nucleotide base pair up to about twenty thousand nucleotide base pairs. The use of the term “amplicon” specifically excludes primer dimers that may be formed in the DNA thermal amplification reaction.

For practical purposes, one should design primers which produce amplicons of a limited size range, for example, between 100 to 1000 bases. Smaller (shorter polynucleotide length) sized amplicons in general are more reliably produced in thermal amplification reactions, allow for shorter cycle times, and can be easily separated and visualized on agarose gels or adapted for use in endpoint TAQMAN®-like assays. Smaller amplicons can be produced and detected by methods known in the art of DNA amplicon detection. In addition, amplicons produced using the primer pairs can be cloned into vectors, propagated, isolated, and sequenced or can be sequenced directly with methods well established in the art. Any primer pair derived from the combination of SEQ ID NO:11 and SEQ ID NO:9 or the combination of SEQ ID NO:12 and SEQ ID NO:9 that are useful in a DNA amplification method to produce an amplicon diagnostic for event MON 95379 or progeny thereof is an aspect of the invention. Any single isolated DNA polynucleotide primer molecule comprising at least 15 contiguous nucleotides of SEQ ID NO:11, or its complement that is useful in a DNA amplification method to produce an amplicon diagnostic for event MON 95379 or progeny thereof is an aspect of the invention. Any single isolated DNA polynucleotide primer molecule comprising at least 15 contiguous nucleotides of SEQ ID NO:12, or its complement that is useful in a DNA amplification method to produce an amplicon diagnostic for plants comprising event MON 95379 or progeny thereof is an aspect of the invention. Any single isolated DNA polynucleotide primer molecule comprising at least 15 contiguous nucleotides of SEQ ID NO:9, or its complement that is useful in a DNA amplification method to produce an amplicon diagnostic for event MON 95379 or progeny thereof is an aspect of the invention.

Polynucleic acid amplification can be accomplished by any of the various polynucleic acid amplification methods known in the art, including the polymerase chain reaction (PCR). Amplification methods are known in the art and are described, inter alia, in U.S. Pat. Nos. 4,683,195 and 4.683.202 and in PCR Protocols: A Guide to Methods and Applications, ed. Innis et al., Academic Press. San Diego, 1990. PCR amplification methods have been developed to amplify up to 22 kb (kilobase) of genomic DNA and up to 42 kb of bacteriophage DNA (Cheng et al., Proc. Natl. Acad. Sci. USA 91:5695-5699, 1994). These methods as well as other methods known in the art of DNA amplification may be used in the practice of the present invention. The sequence of the heterologous DNA insert or flanking genomic DNA sequence from corn event MON 95379 can be verified (and corrected if necessary) by amplifying such DNA molecules from corn seed containing event MON 95379 DNA or corn plants grown from the corn seed containing event MON 95379 DNA deposited with the ATCC having accession No. PTA-125027, using primers derived from the sequences provided herein, followed by standard DNA sequencing of the PCR amplicon or cloned DNA fragments thereof.

The diagnostic amplicon produced by these methods may be detected by a plurality of techniques. One such method is Genetic Bit Analysis (Nikiforov, et al. Nucleic Acid Res. 22:4167-4175, 1994) where a DNA oligonucleotide is designed that overlaps both the adjacent flanking genomic DNA sequence and the inserted DNA sequence. The oligonucleotide is immobilized in wells of a microtiter plate. Following PCR of the region of interest (using one primer in the inserted sequence and one in the adjacent flanking genomic sequence), a single-stranded PCR product can be hybridized to the immobilized oligonucleotide and serve as a template for a single base extension reaction using a DNA polymerase and labeled dideoxynucleotide triphosphates (ddNTPs) specific for the expected next base. Readout may be fluorescent or ELISA-based. A signal indicates presence of the transgene/genomic sequence due to successful amplification, hybridization, and single base extension.

Another method is the Pyrosequencing technique as described by Winge (Innov. Pharma. Tech. 00:18-24, 2000). In this method, an oligonucleotide is designed that overlaps the adjacent genomic DNA and insert DNA junction. The oligonucleotide is hybridized to single-stranded PCR product from the region of interest (one primer in the inserted sequence and one in the flanking genomic sequence) and incubated in the presence of a DNA polymerase. ATP, sulfurylase, luciferase, apyrase, adenosine 5′ phosphosulfate and luciferin. DNTPs are added individually and the incorporation results in a light signal that is measured. A light signal indicates the presence of the transgene/genomic sequence due to successful amplification, hybridization, and single or multi-base extension.

Fluorescence Polarization as described by Chen, et al., (Genome Res. 9:492-498, 1999) is a method that can be used to detect the amplicon of the present invention. Using this method an oligonucleotide is designed that overlaps the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene/genomic sequence due to successful amplification, hybridization, and single base extension.

Real-time Polymerase Chain Reaction (PCR) is the ability to monitor the progress of the PCR as it occurs (i.e., in real time). Data is collected throughout the PCR process, rather than at the end of the PCR. In real-time PCR, reactions are characterized by the point in time during cycling when amplification of a target is first detected rather than the amount of target accumulated after a fixed number of cycles. In a real-time PCR assay, a positive reaction is detected by accumulation of a fluorescent signal. The higher the starting copy number of the nucleic acid target, the sooner a significant increase in fluorescence is observed. The cycle threshold (Ct value) is defined as the number of cycles required for the fluorescent signal to cross the threshold (i.e., exceeds background level). Ct levels are inversely proportional to the amount of target nucleic acid in the sample (i.e., the lower the Ct value, the greater the amount of target nucleic acid in the sample).

Taqman® (PE Applied Biosystems, Foster City, CA) is described as a method of detecting and quantifying the presence of a DNA sequence using real-time PCR and is fully understood in the instructions provided by the manufacturer. Briefly, a FRET oligonucleotide probe is designed that overlaps the genomic flanking and insert DNA junction. The FRET probe and PCR primers (one primer in the insert DNA sequence and one in the flanking genomic sequence) are cycled in the presence of a thermalstable polymerase and dNTPs. Hybridization of the FRET probe results in cleavage and release of the fluorescent moiety away from the quenching moiety on the FRET probe. A fluorescent signal indicates the presence of the transgene/genomic sequence due to successful amplification and hybridization.

Molecular Beacons have been described for use in sequence detection as described in Tyangi, et al. (Nature Biotech.14:303-308, 1996). Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermalstable polymerase and dNTPs. Following successful PCR amplification, hybridization of the FRET probe to the target sequence results in the removal of the probe secondary structure and spatial separation of the fluorescent and quenching moieties. A fluorescent signal results. A fluorescent signal indicates the presence of the flanking/transgene insert sequence due to successful amplification and hybridization.

DNA detection kits that are based on DNA amplification methods contain DNA primer molecules that hybridize specifically to a target DNA and amplify a diagnostic amplicon under the appropriate reaction conditions. The kit may provide an agarose gel based detection method or any number of methods of detecting the diagnostic amplicon that are known in the art. DNA detection kits can be developed using the compositions disclosed herein and are useful for identification of corn event MON 95379 DNA in a sample and can be applied to methods for breeding corn plants containing event MON 95379 DNA. A kit that contains DNA primers that are homologous or complementary to any portion of the corn genomic region as set forth in SEQ ID NO:10 and to any portion of the inserted transgenic DNA as set forth in SEQ ID NO:10 is an object of the invention. The DNA molecules can be used in DNA amplification methods (PCR) or as probes in polynucleic acid hybridization methods, i.e., southern analysis, northern analysis. Kits of the invention may optionally also comprise reagents or instructions for performing the detection or diagnostic reactions described herein.

Probes and primers according to the invention may have complete sequence identity with the target sequence, although primers and probes differing from the target sequence that retain the ability to hybridize preferentially to target sequences may be designed by conventional methods. In order for a nucleic acid molecule to serve as a primer or probe it need only be sufficiently complementary in sequence to be able to form a stable double-stranded structure under the particular solvent and salt concentrations employed. Any conventional nucleic acid hybridization or amplification method can be used to identify the presence of transgenic DNA from corn event MON 95379 in a sample. Probes and primers are generally at least about 11 nucleotides, at least about 18 nucleotides, at least about 24 nucleotides, or at least about 30 nucleotides or more in length. Such probes and primers hybridize specifically to a target DNA sequence under stringent hybridization conditions. Conventional stringency conditions are described by Sambrook et al., 1989, and by Haymes et al., In: Nucleic Acid Hybridization. A Practical Approach. IRL Press. Washington. DC (1985).

Any number of methods well known to those skilled in the art can be used to isolate and manipulate a DNA molecule, or fragment thereof, disclosed in the invention, including thermal amplification methods. DNA molecules, or fragments thereof, can also be obtained by other techniques such as by directly synthesizing the fragment by chemical means, as is commonly practiced by using an automated oligonucleotide synthesizer.

The DNA molecules and corresponding nucleotide sequences provided herein are therefore useful for, among other things, identifying corn event MON 95379, detecting the presence of DNA derived from the transgenic corn event MON 95379 in a sample, and monitoring samples for the presence and/or absence of corn event MON 95379 or plant parts derived from corn plants comprising event MON 95379.

The invention provides corn plants, corn plant cells, corn seeds, corn plant parts (such as pollen, ovule, silk, spike, anther, cob, root tissue, stalk tissue, leaf tissue), corn progeny plants, and corn commodity products. These corn plants, corn plant cells, corn seeds, corn plant parts, corn progeny plants, and corn commodity products contain a detectable amount of a polynucleotide of the invention, e.g., such as a polynucleotide having at least one of the sequences provided as SEQ ID NO:1, SEQ ID NO:2. SEQ ID NO:3, SEQ ID NO:4. SEQ ID NO:5, SEQ ID NO:6, SEQ ID NO:7, SEQ ID NO:8, SEQ ID NO:9, and SEQ ID NO:10. Corn plants, plant cells, seeds, plant parts, and progeny plants of the invention may also contain one or more additional transgenes. Such additional transgene may be any nucleotide sequence encoding a protein or RNA molecule conferring a desirable trait including but not limited to increased insect resistance, increased water use efficiency, increased yield performance, increased drought resistance, increased seed quality, and/or increased herbicide tolerance.

The invention provides corn plants, corn plant cells, corn seeds, corn plant parts (such as pollen, ovule, silk, spike, anther, cob, root tissue, stalk tissue, leaf tissue), corn progeny plants derived from a transgenic corn plant containing event MON 95379 DNA. A representative sample of corn seed containing event MON 95379 DNA has been deposited according to the Budapest Treaty with the American Type Culture Collection (ATCC®). The ATCC repository has assigned the Patent Deposit Designation PTA-125027 to the seed containing event MON 95379 DNA.

The invention provides a microorganism comprising a DNA molecule having at least one sequence selected from SEQ ID NO:1, SEQ ID NO:2, SEQ ID NO:3, SEQ ID NO:4. SEQ ID NO:5, SEQ ID NO:6, SEQ ID NO:7, SEQ ID NO:8. SEQ ID NO:9, and SEQ ID NO:10 present in its genome. An example of such a microorganism is a transgenic plant cell. Microorganisms, such as a plant cell of the invention, are useful in many industrial applications, including but not limited to: (i) use as research tool for scientific inquiry or industrial research; (ii) use in culture for producing endogenous or recombinant carbohydrate, lipid, nucleic acid, or protein products or small molecules that may be used for subsequent scientific research or as industrial products; and (iii) use with modern plant tissue culture techniques to produce transgenic plants or plant tissue cultures that may then be used for agricultural research or production. The production and use of microorganisms such as transgenic plant cells utilizes modern microbiological techniques and human intervention to produce a man-made, unique microorganism. In this process, recombinant DNA is inserted into a plant cell's genome to create a transgenic plant cell that is separate and unique from naturally occurring plant cells. This transgenic plant cell can then be cultured much like bacteria and yeast cells using modern microbiology techniques and may exist in an undifferentiated, unicellular state. The transgenic plant cell's new genetic composition and phenotype is a technical effect created by the integration of the heterologous DNA into the genome of the cell. Another aspect of the invention is a method of using a microorganism of the invention. Methods of using microorganisms of the invention, such as transgenic plant cells, include (i) methods of producing transgenic cells by integrating recombinant DNA into the genome of the cell and then using this cell to derive additional cells possessing the same heterologous DNA; (ii) methods of culturing cells that contain recombinant DNA using modem microbiology techniques; (iii) methods of producing and purifying endogenous or recombinant carbohydrate, lipid, nucleic acid, or protein products from cultured cells; and (iv) methods of using modem plant tissue culture techniques with transgenic plant cells to produce transgenic plants or transgenic plant tissue cultures.

Plants of the invention may pass along the event MON 95379 DNA, including transgene inserted in corn event MON 95379, to progeny. As used herein, “progeny” includes any plant, plant cell, seed, and/or regenerable plant part containing the event MON 95379 DNA derived from an ancestor plant and/or comprising a DNA molecule having at least one sequence selected from SEQ ID NO:1, SEQ ID NO:2. SEQ ID NO:3, SEQ ID NO:4, SEQ ID NO:5. SEQ ID NO:6, SEQ ID NO:7. SEQ ID NO:8, SEQ ID NO:9, and SEQ ID NO:10. Plants, progeny, and seeds may be homozygous or heterozygous for the transgene of event MON 95379. Progeny may be grown from seeds produced by a corn event MON 95379 containing plant and/or from seeds produced by a plant fertilized with pollen from a corn event MON 95379 containing plant.

Progeny plants may be self-pollinated (also known as “selfing”) to generate a true breeding line of plants, i.e., plants homozygous for the transgene. Selfing of appropriate progeny can produce plants that are homozygous for both added exogenous genes.

Alternatively, progeny plants may be out-crossed. e.g., bred with another unrelated plant, to produce a varietal or a hybrid seed or plant. The other unrelated plant may be transgenic or non-transgenic. A varietal or hybrid seed or plant of the invention may thus be derived by sexually crossing a first parent that lacks the specific and unique DNA of the corn event MON 95379 with a second parent comprising corn event MON 95379, resulting in a hybrid comprising the specific and unique DNA of the corn event MON 95379. Each parent can be a hybrid or an inbred/varietal, so long as the cross or breeding results in a plant or seed of the invention, i.e., a seed having at least one allele containing the DNA of corn event MON 95379 and/or a DNA molecule having at least one sequence selected from SEQ ID NO:1, SEQ ID NO:2, SEQ ID NO:3, SEQ ID NO:4, SEQ ID NO:5, SEQ ID NO:6, SEQ ID NO:7. SEQ ID NO:8, SEQ ID NO:9, and SEQ ID NO:10. Two different transgenic plants may thus be crossed to produce hybrid offspring that contain two independently segregating, added, exogenous genes. For example, event MON 95379 containing Cry1B.868 and Cry1Da_7 conferring insect resistance to corn can be crossed with other transgenic corn plants to produce a plant having the characteristics of both transgenic parents. One example of this would be a cross of event MON 95379 containing Cry1B.868 and Cry1 Da_7 conferring Lepidopteran resistance to corn with a plant having one or more additional traits such as herbicide tolerance, insect resistance, or drought tolerance, resulting in a progeny plant or seed that has resistance to Lepidopteran insect pests and has at least one or more additional traits. Back-crossing to a parental plant and out-crossing with a non-transgenic plant are also contemplated, as is vegetative propagation. Descriptions of other breeding methods that are commonly used for different traits and crops can be found in one of several references, e.g., Fehr, in Breeding Methods for Cultivar Development, Wilcox J. ed., American Society of Agronomy. Madison WI (1987).

Plants, progeny, seeds, cells and plant parts of the invention may also contain one or more additional corn trait(s) or transgenic events, particularly those introduced by crossing a corn plant containing corn event MON 95379 with another corn plant containing the additional trait(s) or transgenic events. Such trait(s) or transgenic events include, but are not limited to, increased insect resistance, herbicide tolerance, increased water use efficiency, increased yield performance, increased drought resistance, increased seed quality, improved nutritional quality, hybrid seed production, or disease or fungal resistance. Corn transgenic events are known to those of skill in the art. For example, a list of such traits is provided by the United States Department of Agriculture's (USDA) Animal and Plant Health Inspection Service (APHIS) and can be found on their website: www.aphis.usda.gov. Two or more transgenic events may thus be combined in a progeny seed or plant by crossing two parent plants each comprising one or more transgenic events, collecting the progeny seed, and selecting for progeny seed or plants that contain the two or more transgenic events. These steps may be repeated until the desired combination of transgenic events in a progeny is achieved. Back-crossing to a parental plant and out-crossing with a non-transgenic plant are also contemplated, and is vegetative propagation.

The invention provides a plant part that is derived from corn plants comprising event MON 95379. As used herein, a “plant part” refers to any part of a plant which is comprised of material derived from a corn plant comprising event MON 95379. Plant parts include but are not limited to pollen, ovule, silk, spike, anther, cob, root tissue, stalk tissue, and leaf tissue. Plant parts may be viable, nonviable, regenerable, and/or nonregenerable.

The invention provides a commodity product that is derived from corn plants comprising event MON 95379 and that contains a detectable amount of a nucleic acid specific for event MON 95379. As used herein, a “commodity product” refers to any composition or product which is comprised of material derived from a corn plant, whole or processed corn seed, or one or more plant cells and/or plant parts containing the corn event MON 95379 DNA. Nonviable commodity products include but are not limited to nonviable seeds, whole or processed seeds, seed parts, and plant parts; animal feed comprising corn, corn oil, corn meal, corn flour, corn flakes, corn bran, pasta made with corn, corn biomass, and fuel products produced using corn and corn parts. Viable commodity products include but are not limited to seeds, plants, and plant cells. The corn plants comprising event MON 95379 can thus be used to manufacture any commodity product typically acquired from corn. Any such commodity product that is derived from corn plants comprising event MON 95379 may contain at least a detectable amount of the specific and unique DNA corresponding to corn event MON 95379, and specifically may contain a detectable amount of a polynucleotide comprising a DNA molecule having at least one sequence selected from SEQ ID NO:1. SEQ ID NO:2. SEQ ID NO:3. SEQ ID NO:4. SEQ ID NO:5. SEQ ID NO:6. SEQ ID NO:7. SEQ ID NO:8, SEQ ID NO:9, and SEQ ID NO:10. Any standard method of detection for nucleotide molecules may be used, including methods of detection disclosed herein. A commodity product is with the scope of the invention if there is any detectable amount of a DNA molecule having at least one sequence selected from SEQ ID NO:1, SEQ ID NO:2, SEQ ID NO:3, SEQ ID NO:4, SEQ ID NO:5, SEQ ID NO:6. SEQ ID NO:7, SEQ ID NO:8, SEQ ID NO:9, and SEQ ID NO:10 in the commodity product.

The corn plants, corn plant cells, corn seeds, corn plant parts (such as pollen, ovule, silk, spike, anther, cob, root tissue, stalk tissue, leaf tissue), corn progeny plants, and commodity products of the invention are therefore, useful for, among other things, growing plants for the purpose of producing seed and/or plant parts comprising corn event MON 95379 for agricultural purposes, producing progeny comprising corn event MON 95379 for plant breeding and research purposes, use with microbiological techniques for industrial and research applications, and sale to consumers.

Methods for producing an insect resistant corn plant comprising the DNA sequences specific and unique to event MON 95379 of the invention are provided. Transgenic plants used in these methods may be homozygous or heterozygous for the transgene. Progeny plants produced by these methods may be varietal or hybrid plants; may be grown from seeds produced by corn event MON 95379 containing plant and/or from seeds produced by a plant fertilized with pollen from a corn event MON 95379 containing plant; and may be homozygous or heterozygous for the transgene. Progeny plants may be subsequently self-pollinated to generate a true breeding line of plants, i.e., plants homozygous for the transgene, or alternatively may be out-crossed, e.g., bred with another unrelated plant, to produce a varietal or a hybrid seed or plant.

Methods of detecting the presence of DNA derived from a corn cell, corn tissue, corn seed, or corn plant comprising corn event MON 95379 in a sample are provided. One method consists of (i) extracting a DNA sample from at least one corn cell, corn tissue, corn seed, or corn plant; (ii) contacting the DNA sample with at least one primer that is capable of producing DNA sequence specific to event MON 95379 DNA under conditions appropriate for DNA sequencing; (iii) performing a DNA sequencing reaction; and then (iv) confirming that the nucleotide sequence comprises a nucleotide sequence specific for event MON 95379, of the construct comprised therein, such as one selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Another method consists of (i) extracting a DNA sample from at least one corn cell, corn tissue, corn seed, or corn plant; (ii) contacting the DNA sample with a primer pair that is capable of producing an amplicon from event MON 95379 DNA under conditions appropriate for DNA amplification; (iii) performing a DNA amplification reaction; and then (iv) detecting the amplicon molecule and/or confirming that the nucleotide sequence of the amplicon comprises a nucleotide sequence specific for event MON 95379, such as one selected from the group consisting of SEQ ID NO:1. SEQ ID NO:2, SEQ ID NO:3, SEQ ID NO:4, SEQ ID NO:5, SEQ ID NO:6, SEQ ID NO:7. SEQ ID NO:8. SEQ ID NO:9, and SEQ ID NO:10. The amplicon should be one that is specific for event MON 95379, such as an amplicon that comprises SEQ ID NO:1, or SEQ ID NO:2, or SEQ ID NO:3, or SEQ ID NO:4, or SEQ ID NO:5, or SEQ ID NO:6, or SEQ ID NO:7, or SEQ ID NO:8, or SEQ ID NO:9, or SEQ ID NO:10. The detection of a nucleotide sequence specific for event MON 95379 in the amplicon is determinative and/or diagnostic for the presence of the corn event MON 95379 specific DNA in the sample. An example of a primer pair that is capable of producing an amplicon from event MON 95379 DNA under conditions appropriate for DNA amplification is provided as SEQ ID NO:15 and SEQ ID NO:16.

Other primer pairs may be readily designed by one of skill in the art and would produce an amplicon comprising SEQ ID NO:1, or SEQ ID NO:2, or SEQ ID NO:3, or SEQ ID NO:4, or SEQ ID NO:5, or SEQ ID NO:6, or SEQ ID NO:7, or SEQ ID NO:8 wherein such a primer pair comprises at least one primer within the genomic region flanking the insert and a second primer within the insert. Another method of detecting the presence of DNA derived from a corn cell, corn tissue, corn seed, or corn plant comprising corn event MON 95379 in a sample consists of (i) extracting a DNA sample from at least one corn cell, corn tissue, corn seed, or corn plant; (ii) contacting the DNA sample with a DNA probe specific for event MON 95379 DNA; (iii) allowing the probe and the DNA sample to hybridize under stringent hybridization conditions; and then (iv) detecting hybridization between the probe and the target DNA sample. An example of the sequence of a DNA probe that is specific for event MON 95379 is provided as SEQ ID NO:17. Other probes may be readily designed by one of skill in the art and would comprise at least one fragment of genomic DNA flanking the insert and at least one fragment of insert DNA such as the sequence provided in SEQ ID NO:1, SEQ ID NO:2, SEQ ID NO:3, SEQ ID NO:4. SEQ ID NO:5. SEQ ID NO:6, SEQ ID NO:7, SEQ ID NO:8, and SEQ ID NO:10. Detection of probe hybridization to the DNA sample is diagnostic for the presence of corn event MON 95379 specific DNA in the sample. Absence of hybridization is alternatively diagnostic of the absence of corn event MON 95379 specific DNA in the sample.

DNA detection kits are provided that are useful for the identification of corn event MON 95379 DNA in a sample and can also be applied to methods for breeding corn plants containing the appropriate event DNA. Such kits contain DNA primers and/or probes comprising fragments of SEQ ID NO:1, SEQ ID NO:2, SEQ ID NO:3. SEQ ID NO:4, SEQ ID NO:5, SEQ ID NO:6, SEQ ID NO:7. SEQ ID NO:8. SEQ ID NO:9, and SEQ ID NO:10. One example of such a kit comprises at least one DNA molecule of sufficient length of continuous nucleotides of SEQ ID NO:10 to function as a DNA probe useful for detecting the presence and/or absence of DNA derived from transgenic corn plants comprising event MON 95379 in a sample. The DNA derived from transgenic corn plants comprising event MON 95379 would comprise a DNA molecule having at least one sequence selected from SEQ ID NO:1. SEQ ID NO:2, SEQ ID NO:3. SEQ ID NO:4. SEQ ID NO:5. SEQ ID NO:6, SEQ ID NO:7, SEQ ID NO:8, SEQ ID NO:9, and SEQ ID NO:10. A DNA molecule sufficient for use as a DNA probe is provided that is useful for determining, detecting, or diagnosing the presence and/or absence of corn event MON 95379 DNA in a sample is provided as SEQ ID NO: 17. Other probes may be readily designed by one of skill in the art and should comprise at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 contiguous nucleotides of SEQ ID NO:10 and be sufficiently unique to corn event MON 95379 DNA in order to identify DNA derived from the event.

Another type of kit comprises a primer pair useful for producing an amplicon useful for detecting the presence and/or absence of DNA derived from transgenic corn event MON 95379 in a sample. Such a kit would employ a method comprising contacting a target DNA sample with a primer pair as described herein, then performing a nucleic acid amplification reaction sufficient to produce an amplicon comprising a DNA molecule having at least one sequence selected from SEQ ID NO:1, SEQ ID NO:2, SEQ ID NO:3, SEQ ID NO:4, SEQ ID NO:5, SEQ ID NO:6. SEQ ID NO:7. SEQ ID NO:8. SEQ ID NO:9, and SEQ ID NO:10 and then detecting the presence and/or absence of the amplicon. Such a method may also include sequencing the amplicon or a fragment thereof, which would be determinative of, i.e., diagnostic for, the presence of the corn event MON 95379 specific DNA in the target DNA sample. Other primer pairs may be readily designed by one of skill in the art and should comprise at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of sequences provided in, but not limited to SEQ ID NO:1, SEQ ID NO:2. SEQ ID NO:3. SEQ ID NO:4. SEQ ID NO:5, SEQ ID NO:6, SEQ ID NO:7. SEQ ID NO:8, SEQ ID NO:9, and SEQ ID NO:10, and be sufficiently unique to corn event MON 95379 DNA in order to identify DNA derived from the event.

The kits and detection methods of the invention are useful for, among other things, identifying corn event MON 95379, selecting plant varieties or hybrids comprising corn event MON 95379, detecting the presence of DNA derived from the transgenic corn plant comprising event MON 95379 in a sample, and monitoring samples for the presence and/or absence of corn plants comprising event MON 95379, or plant parts derived from corn plants comprising event MON 95379.

The sequences of the heterologous DNA insert, junction sequences, or flanking sequence from corn event MON 95379 can be verified (and corrected if necessary) by amplifying such sequences from the event using primers derived from the sequences provided herein followed by standard DNA sequencing of the amplicon or of the cloned DNA.

Methods of detecting the zygosity of the transgene allele of DNA derived from a corn cell, corn tissue, corn seed, or corn plant comprising corn event MON 95379 in a sample are provided. One method consists of (i) extracting a DNA sample from at least one corn cell, corn tissue, corn seed, or corn plant; (ii) contacting the DNA sample with a primer pair that is capable of producing a first amplicon diagnostic for event MON 95379; (iii) contacting the DNA sample with a primer pair that is capable of producing a second amplicon diagnostic for native corn genomic DNA not comprising event MON 95379; (iv) performing a DNA amplification reaction; and then (v) detecting the amplicons, wherein the presence of only the first amplicon is diagnostic of a homozygous event MON 95379 DNA in the sample, and the presence of both the first amplicon and the second amplicon is diagnostic of a corn plant heterozygous for event MON 95379 allele. An exemplary set of primers pairs are presented as SEQ ID NO:15 and SEQ ID NO:16 which produce an amplicon diagnostic for event MON 95379; and SEQ ID NO:15 and SEQ ID NO:27 which produces an amplicon diagnostic for non-inserted wild-type corn genomic DNA not comprising event MON 95379. A set of probes can also be incorporated into such an amplification method to be used in a real-time PCR format using the primer pair sets described above. An exemplary set of probes are presented as SEQ ID NO:17 (diagnostic for the amplicon for the event MON 95379) and SEQ ID NO:28 (diagnostic for the amplicon for wild-type corn genomic DNA not comprising event MON 95379).

Another method for determining zygosity consists of (i) extracting a DNA sample from at least one corn cell, corn tissue, corn seed, or corn plant; (ii) contacting the DNA sample with a probe set which contains at least a first probe that specifically hybridizes to event MON 95379 DNA and at least a second probe that specifically hybridizes to corn genomic DNA that was disrupted by insertion of the heterologous DNA of event MON 95379 and does not hybridize to event MON 95379 DNA; (iii) hybridizing the probe set with the sample under stringent hybridization conditions, wherein detecting hybridization of only the first probe under the hybridization conditions is diagnostic for a homozygous allele of event MON 95379 DNA in the sample; and wherein detecting hybridization of both the first probe and the second probe under the hybridization conditions is diagnostic for a heterozygous allele of event MON 95379 in a DNA sample.

Yet another method for determining zygosity consists of (i) extracting a DNA sample from at least one corn cell, corn tissue, corn seed, or corn plant; (ii) contacting the DNA sample with a primer pair that is capable of producing an amplicon of one of the toxin coding sequences encoding Cry1B.868 or Cry1Da_7; (iii) contacting the DNA sample with a primer pair that is capable of producing an amplicon of an internal standard known to be single-copy and homozygous in the corn plant; (iv) contacting the DNA sample with a probe set which contains at least a first probe that specifically hybridizes to one of the toxin coding sequences encoding Cry1B.868 or Cry1Da_7, and at least a second probe that specifically hybridizes to the internal standard genomic DNA known to be single-copy and homozygous in the corn plant; (v) performing a DNA amplification reaction using real-time PCR and determining the cycle thresholds (Ct values) of the amplicon corresponding to the toxin coding sequence and the single-copy, homozygous internal standard; (vi) calculating the difference (ΔCt) between the Ct value of the single-copy, homozygous internal standard amplicon and the Ct value of the toxin coding sequence amplicon; and (vii) determining zygosity, wherein a ΔCt of around zero (0) indicates homozygosity of the inserted T-DNA and a ΔCt of around one (1) indicates heterozygosity of the inserted T-DNA. Heterozygous and homozygous events are differentiated by a ΔCt value unit of approximately one (1). Given the normal variability observed in real-time PCR due to multiple factors such as amplification efficiency and ideal annealing temperatures, the range of “about one (1)” is defined as a ΔCt of 0.75 to 1.25. Primer pairs and probes for the above method for determining zygosity can either amplify and detect amplicons from the Cry1B.868 coding sequence and internal standard, or amplify and detect amplicons from the Cry1Da_7 coding sequence and internal standard. Exemplary primer pairs for the detection of the amplicons corresponding to the Cry1B.868 coding sequence and internal standard are presented as SEQ ID NO:18 combined with SEQ ID NO:19 (internal standard) and SEQ ID NO:21 combined with SEQ ID NO:22 (Cry1B.868). The accompanying exemplary probes are presented as SEQ ID NO:20 (internal standard) and SEQ ID NO:23 (Cry1B.868). Exemplary primer pairs for the detection of the amplicons corresponding to the Cry1Da_7 coding sequence and internal standard are presented as SEQ ID NO:18 combined with SEQ ID NO:19 (internal standard) and SEQ ID NO:24 combined with SEQ ID NO:25 (Cry1Da_7). The accompanying exemplary probes are presented as SEQ ID NO:20 (internal standard) and SEQ ID NO:26 (Cry1 Da_7).

### DEPOSIT INFORMATION

A deposit of a representative sample of corn seed containing event MON 95379 was made on Apr. 20, 2018 according to the Budapest Treaty with the American Type Culture Collection (ATCC) having an address at 10801 University Boulevard. Manassas. Virginia USA. Zip Code 20110, and assigned ATCC Accession No. PTA-125027. Access to the deposits will be available during the pendency of the application to the Commissioner of Patents and Trademarks and persons determined by the Commissioner to be entitled thereto upon request. Upon issuance of the patent, all restrictions upon availability to the public will be irrevocably removed. The deposit will be maintained in the depository for a period of thirty (30) years, or five (5) years after the last request, or for the effective life of the patent, whichever is longer, and will be replaced as necessary during that period.

### EXAMPLES

The following Examples are included to more fully describe the invention. Summarized are the construction and testing of one hundred and twenty-five (125) constructs, the production of about ten thousand seven hundred and eighty-five (10.785) events (both proof of concept and commercial), and the analysis of hundreds of thousands of individual plants over six (6) years through the rigorous molecular, agronomic, and field testing required for the creation and selection of corn event MON 95379.

The Examples demonstrate certain preferred embodiments of the invention. It should be appreciated by those of skill in the art that the techniques disclosed in the Examples that follow represent approaches the inventors have found function well in the practice of the invention, and thus can be considered to constitute examples of preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

### Expression Cassette Testing, Construct Design, Plant Testing and Construct Selection

Transgene expression in plants is influenced by numerous different factors. The right combination of insecticidal proteins and different expression elements driving expression in plants, while not resulting in off-phenotypes, must be found. Further, beyond the expression elements themselves and their combination and orientation in a cassette, the expression of transgenes in plants is known to be influenced by chromosomal insertion position, perhaps due to chromatin structure (e.g., heterochromatin) or the proximity of transcriptional regulation elements (e.g., enhancers) close to the integration site (Kurt Weising et al., (1988) Foreign genes in plants: transfer, structure, expression and applications. Annu. Rev. Genet. 22: 421-77). For example, it has been observed in plants and in other organisms that there may be wide variation in the levels of expression of an introduced gene from the same construct among events with different chromosomal insertion positions. Different chromosomal insertion positions may also produce differences in spatial or temporal patterns of expression, that may not correspond to the patterns expected from transcriptional regulatory elements present in the introduced gene construct.

For these reasons, it is often necessary to create and screen a large number of constructs and transformation events in order to identify a construct, and then an event, which demonstrates optimal expression of the introduced genes of interest, while also not producing agronomic or phenotypic off-types.

For these reasons, the development of a transgenic corn plant comprising insecticidal proteins that were active against Lepidopterans without any negative effects on agronomics, yield, or stacking viability required extensive research, development, and analysis. Specifically, over a six (6) year period, approximately ten thousand, seven hundred eight-five (10,785) proof of concept and commercial transgenic events derived from one hundred twenty-five (125) different plasmid vector constructs were developed, tested, and analyzed.

This Example describes the design and testing in corn plants of one hundred and twenty five (125) different constructs to identify the preferred construct for event creation. Each construct varied with respect to the coding sequences for the insecticidal proteins and the transcriptional regulatory elements. Testing was done to select the best construct for use in expressing the insecticidal proteins in plants. Each construct had a unique configuration, varying by expression cassette composition (both insecticidal proteins and expression elements), orientation, and whether or not proteins were targeted to the chloroplast.

In an initial proof of concept and developmental stage, one hundred seventeen (117) constructs comprising different combinations of twenty-six (26) distinct promoters, twenty-six (26) distinct introns, and ten (10) distinct insect toxin coding sequences, were used to generate approximately six thousand (6,000) transformed events. After initial molecular characterization for the presence of the transgene(s), five thousand fifty-two (5,052) single and double-copy transformed corn events were selected for further characterization and efficacy testing. These events were evaluated for phenotypic or agronomic off-types, the level of expression of the insect toxin proteins, and efficacy against selected Lepidopteran insect pest species. The resulting efficacy and protein expression data, along with any information regarding phenotypic and agronomic off-types was used to eliminate inefficacious proteins, expression elements and combinations, and was used to design a smaller number of binary commercial transformation plasmid constructs to be used in the next phase of development.

In the next phase of development, eight (8) new constructs were created. These constructs comprised combinations of two (2) to four (4) insect toxin transgene expression cassettes in different orientations (convergent or divergent). These eight (8) constructs were used to generate a total of five thousand seven hundred thirty-three (5,733) transformed events (also referred to as “transformants”). After shoot formation in culture, a subset of the transformed events were selected based upon visual characteristics and early molecular analysis. A total of eight hundred twenty-three (823) transformed events were selected and transplanted to pots and grown for further study.

The resulting R₀ generation transformed events were analyzed for efficacy against selected Lepidopteran species, toxin protein expression, plant health, seed return, and phenotypic and agronomic off-types. The R₀ generation events were also characterized molecularly to ensure cassette intactness and proper insertion in the corn genome. Many of the events were dropped from testing due to failure to pass the agronomic analysis and molecular characterization testing. In addition, one (1) of the eight (8) constructs was dropped from further study at this R₀ stage because it produced events with off-phenotypes. In addition to these agronomic problems, later mode of action (“MOA”) studies conducted demonstrated that an insect toxin protein contained in this construct demonstrated an overlapping MOA to a commercially-available protein.

Mode of Action studies were conducted on one of the insect proteins common to four (4) of the eight (8) constructs. These studies demonstrated that this insect protein had an overlapping MOA to a commercially-available protein. Proteins that demonstrate an analogous or overlapping MOA to a currently utilized commercial insecticidal protein are not desirable because of resistance development, which could render a protein with a similar MOA ineffective against insect populations. As such, these four (4) constructs, and the events arising therefrom, were dropped. As noted previously, one (1) of the four (4) dropped constructs also produced events with off-phenotypes at the R₀ stage.

In the next stage of development, one hundred fifty (150) events derived from the remaining four (4) constructs were further evaluated at the F₁(heterozygous hybrid)/R₁ (homozygous inbred) and R₂ generation for efficacy, seed return and segregation, phenotypic and agronomic off-types, and further molecular characterization. Two (2) constructs from the remaining four (4) constructs were dropped from further study in this stage for failure to meet one or more of the criteria for advancement, leaving events derived from (2) constructs for further evaluation.

Seventy-seven (77) events, forty-one (41) derived from the construct used to generate event MON 95379 (“Construct MON95”) and thirty-six (36) events derived from the other construct (“Construct 1”), were evaluated as R₂ inbreds and F₁ hybrids for efficacy, seed return and segregation, phenotypic and agronomic off-types and further molecular characterization. Based upon the results of these evaluations, events associated with Construct 1 were de-prioritized, shelved and stored.

Thus, numerous rounds of testing and comparison of various constructs revealed that the transgene cassette provided as SEQ ID NO:13, Construct MON95, was the best option for efficacy against the Lepidopteran pest species Fall Armyworm (FAW, Spodoptera frugiperda), Corn Earworm (CEW. Helicoverpa zea), Southwestern Corn Borer (SWCB, Diatraea grandiosella). Surgarcane Borer (SCB, Diatraea saccharalis), and Lesser Cornstalk Borer (LSCB, Elasmopalpus lignosellus), with the best molecular characterization and agronomic performance.

Table 2 illustrates the number of transformed events derived (“Plugged”), the number of transformed events selected for growth as R0 events (“Transplanted”), and the points at which each respective construct was dropped in the evaluation, research and development process that led to the selection of Construct MON95.

TABLE 2
Event construct selection.

| Construct | Plugged | Transplanted | Dropped |
|---|---|---|---|
| Construct MON 95 | 1202 | 210 | |
| Construct 1 | 799 | 173 | Shelved based |
| | | | on R₂ and F₁ |
| | | | data |
| Construct 2 | 679 | 113 | R₂/F1/R₁ |
| Construct 3 | 1344 | 20 | MOA/R₀ |
| Construct 4 | 232 | 41 | R₂/F₁/R₁ |
| Construct 5 | 544 | 88 | MOA |
| Construct 6 | 584 | 95 | MOA |
| Construct 7 | 349 | 83 | MOA |

### Example 2

### Field Trials, Molecular Testing and Event Selection

This Example describes the molecular characterization, analysis, and testing in field trials of events created with Construct MON95 in multiple locations over several years, which lead to the selection of the final event, MON 95379.

Table 3 illustrates the process used to select the final event, MON 95379. At the commercial transformation R₀ screen, two hundred ten (210) R₀ transformed events from Construct MON95 were derived and selected for growth. Of the initial two hundred ten (210) selected R₀ transformed events, one hundred forty-seven events (147) were dropped due to concerns regarding efficacy, protein expression, seed return and plant health, or molecular characterization. This left sixty-three (63) events for assay and testing in the next stage of development, the F₁ Screen and the R₁ Nursery stage. In this stage, eleven (11) events were dropped due to efficacy concerns in the greenhouse testing. Another three (3) events were dropped because of insufficient return of seed from the nursery and/or segregation analysis of the resulting seed. Finally, another five (5) events were removed due to issues discovered in molecular characterization and three (3) events were removed due to issues discovered in molecular southern analysis, leaving forty-one (41) events for assay in the next generation. At the R₂/F₁ stage of testing, two (2) of the remaining forty-one (41) events were dropped due to issues discovered in further molecular southern characterization, leaving thirty-nine (39) events.

The remaining thirty-nine (39) events were advanced in two different concurrent parallel testing stages: 1) further field trials' and 2) Cre-excision of the selection cassette and the production of gold standard seed. Events were dropped in each of these concurrent parallel testing stages.

During Cre-excision, eleven (11) events were dropped due to issues discovered in molecular characterization after cre-excision of the glyphosate selection cassette. Further, another six (6) events were dropped due to issues discovered in molecular characterization during gold standard seed production.

During the concurrent field testing, based on data collected from the 2016 U.S. Field Trails, another four (4) events were dropped due to efficacy concerns and another twelve (12) events were dropped due to agronomic concerns. Then, based on data collected from the Brazil Field Trials, another event was dropped due to efficacy concerns. Next, bioinformatic analysis conducted during the 2017 U.S. Field Trials resulted in the removal of another three (3) events from further testing, leaving two events: Event 1 and MON 95379. After further analysis ofthe agronomics of the events from multiple field trials in the U.S., Brazil, Argentina and Puerto Rico, event MON 95379 was selected as the event for commercialization because it ranked higher than Event 1 when all the characteristics of molecular characterization, protein expression, efficacy and agronomics of each event were compared.

TABLE 3
MON 95379 event selection.

| | | | Events |
|---|---|---|---|
| | | Events | Remaining |
| Stage | Assay | Removed | 210 |
| Comm. TFN R₀ | Efficacy | 21 | 63 |
| Screen | Expression | 6 | |
| | Seed Return/plant health | 51 | |
| | Molecular characterization | 69 | |
| R₁/F₁ | Greenhouse Efficacy | 11 | 41 |
| | Molecular characterization | 5 | |
| | Nursery return/segregation | 3 | |
| | Molecular southern | 3 | |
| R₂/F₁ | Molecular southern | 2 | 39 |
| Cre Excision | Molecular characterization | 11 | 28 |
| | after CP4 excision | | |
| Gold Standard | Molecular characterization | 6 | 22 |
| Seed | | | |
| U.S. Field 2016 | Efficacy | 4 | 6 |
| | Agronomics | 12 | |
| Brazil Field | Efficacy | 1 | 5 |
| U.S. Field 2017 | Bioinformatic molecular | 3 | 2 |
| | analysis | | |
| Commercial | Further analysis of molecular | 1 | MON 95379 |
| Selection | characterization, protein | | |
| | expression, efficacy | | |
| | and agronomics from | | |
| | multiple field trials | | |

### Example 3

### Cre-excision of the Glyphosate Selection Cassette in Corn Event MON 95379

This Example describes the removal of the glyphosate selection cassette from corn event MON 95379 through in vivo Cre-excision. The glyphosate selection cassette was used to select transformed events. By removal of the selection cassette, a “marker-free” event was created wherein only the insecticidal protein expression cassettes remained in the final event.

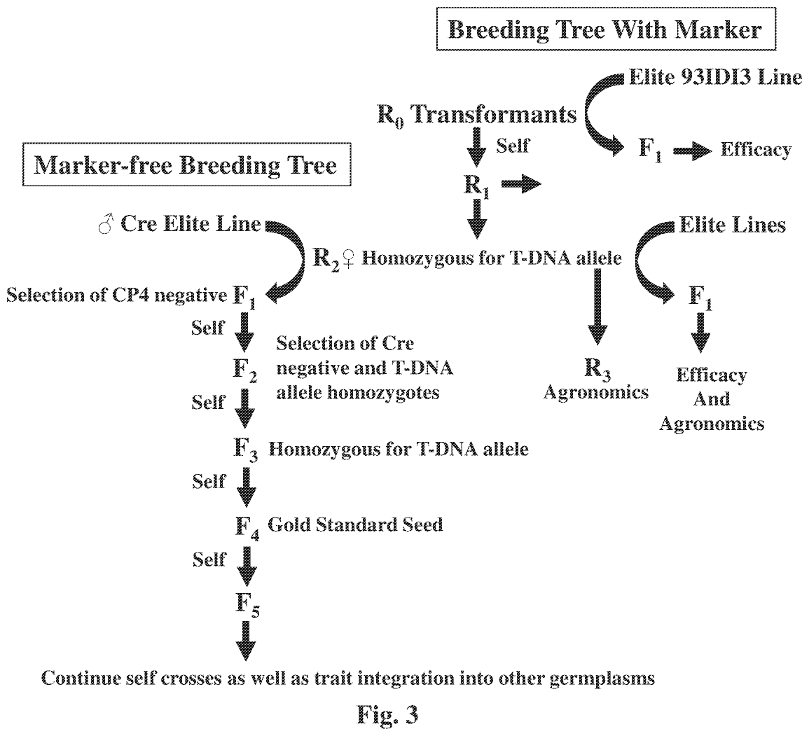

FIG. 3 illustrates the breeding process used to generate the marker-free event MON 95379 corn event. Corn variety LH244 immature embryos were transformed using an Agrobacterium-mediated transformation process with Construct MON95 (presented as SEQ ID NO:13, and illustrated in FIG. 2). Construct MON95 comprises three (3) expression cassettes: two (2) expression cassettes for the expression of the insecticidal proteins Cry1B.868 and Cry1Da_7, and a single cassette used for the selection of transformed plant cells using glyphosate selection. The selection cassette was flanked on both sides with LoxP Cre-recombinase recognition sites.

After transformation, the R₀ transformants were self-pollinated for two (2) generations, during which time many events were removed based upon various assays such as efficacy, protein expression, seed return and plant health, and molecular characterization. By the R₂ generation, thirty-nine (39) events remained from the initial two hundred ten (210) events. The thirty-nine (39) homozygous R₂ generation events were bred with an elite line of transformed corn plants expressing Cre-recombinase enzyme, derived from Enterobacteria phage P1.

This stage in which R₂ generation events were bred with plants expressing Cre-recombinase is identified as “Cre Cross”. Specifically in this stage, de-tasseled (female) R₂ generation plants homozygous for SEQ ID NO:13 were cross-pollinated with transgenic corn plants (male) homozygous for a transgene cassette used for expression of Cre-recombinase enzyme. The Cre-recombinase expressing male donor pollen germinates after landing on the silk tissue of the female plant comprising SEQ ID NO:13. Once the pollen tube enters the embryo sac, the pollen tube ruptures, setting free the two sperms of the Cre-recombinase expressing male donor. The nucleus of one sperm fuses with the egg nucleus, forming the zygote. The other sperm nucleus fuses with one of the two polar nuclei which in turn fuses with the other polar nucleus, thereby establishing the primary endosperm nucleus.

Thus, in using the Cre-recombinase expressing plant as the male pollen donor, both the embryo and endosperm of the resulting cross will express Cre-recombinase as the cells divide and develop and become a corn kernel (i.e., seed). The Cre-recombinase binds to inverted repeats in the LoxP site and catalyzes a crossover in an eight-base pair spacer region of the two LoxP sites that flank the expression cassette, resulting in the excision of the marker cassette with one LoxP site remaining in the integrated T-DNA due to recombination (see FIG. 2, “Inserted T-DNA After Cre-Excision”).

The F₁ progeny resulting from the Cre Cross were selected for the absence of the CP4 selection cassette and allowed to self-pollinate. Through this process, the two alleles—the Cre-recombinase allele and the allele for the T-DNA used to generate event MON 95379—segregate in the resulting F2 population, resulting in progeny homozygous or heterozygous for one or both alleles.

The F2 progeny which demonstrated the absence of the Cre-recombinase allele and homozygosity for SEQ ID NO:9, the transgenic inserted T-DNA after Cre-excision, were selected. These selected F2 progeny were self-pollinated, giving rise to an F3 generation homozygous for SEQ ID NO:9.

A further self-pollination resulted in F3 progeny seed (F4 seed) which were assayed for purity, and were designated as “Gold Standard Seed.” F4 was the first generation of gold standard seed.

Excision of the glyphosate selection marker cassette did not affect the expression of Cry1B.868 and Cry1Da_7. Removing the glyphosate selection cassette from corn event MON 95379 through Cre-excision provided a transgenic corn event which is resistant to Lepidopteran pests without adding tolerance to glyphosate in the final event. This “marker-free” event assures flexibility when building corn breeding stacks with other corn transgenic events to provide a multiplicity of products incorporating event MON 95379 and allowing multiple options for providing additional traits in the final breeding stacks.

### Example 4

### Corn Event MON 95379 Demonstrates Resistance to the Lepidopteran Insect Pests Fall Armyworm, Corn Earworm, Southwestern Corn Borer, Sugarcane Borer

This Example describes the activity of the MON 95379 event against Lepidopteran insect pests. The insect toxin proteins Cry1B.868 and Cry1 Da_7, when expressed together in corn event MON 95379, provide resistant to Fall Armyworm (Spodoptera frugiperda), Corn Earworm (Helicoverpa zea), Southwestern Corn Borer (Diatraea grandiosella), and Surgarcane Borer (Diatraea saccharalis).

After transformation and insertion of Construct MON95, forty-one (41) R₀ events were selected for bioassay using leaf discs. Bioassays using plant leaf disks were performed analogous to those described in U.S. Pat. No. 8,344,207. A non-transformed LH244 corn plant was used to obtain tissue to be used as a negative control. Plates comprising wells with one insect per leaf disc in each well were incubated for three (3) days. After three (3) days, the plates were examined. If at least fifty percent (50%) of the leaf disc in the negative controls was consumed, measurements were taken of the transgenic event leaf discs. If less than fifty percent (50%) of the leaf discs in the negative controls had not yet been consumed, the insects were allowed to continue feeding until the fifty percent (50%) target was achieved. Measurements of leaf damage (“leaf damage ratings” or “LDR”) and mortality were taken for each well. An average of each measure was determined. The leaf damage ratings ranged from one (1) to eleven (11) and reflect a percentage of the consumed leaf disc. Table 4 shows the leaf damage rating scale used for the R₀ leaf disc assays. On this rating scale, the negative controls will always have an LDR of at least 10.

TABLE 4
Leaf Damage Ratings (LDR) scale for R₀ leaf disc assays.

| | Amount of |
|---|---|
| Leaf Damage | feeding |
| Rating (LDR) | damage |
| 1 | ≤5% |
| 2 | ≤10% |
| 3 | ≤15% |
| 4 | ≤20% |
| 5 | ≤25% |
| 6 | ≤30% |
| 7 | ≤35% |
| 8 | ≤40% |
| 9 | ≤45% |
| 10 | ≤50% |
| 11 | >50% |

Table 5 shows the mean leaf damage ratings for the forty-one (41) events transformed with Construct MON95, including the MON 95379 event. As can be seen in Table 5, expression of the two insecticidal proteins, Cry1B.868 and Cry1Da_7, provided resistance to Fall Armyworm (FAW). Corn Earworm (CEW), and Southwestern Corn Borer (SWCB). The LDRs for the negative controls were between 10 and 11. The FAW and SWCB consumed only approximately five percent (5%) of the event MON 95379 leaf disc in comparison to the negative controls which consumed at least fifty percent (50%) of the leaf disc. With respect to CEW, only approximately 6.25% of the leaf discs were consumed in comparison to the negative controls which consumed at least fifty percent (50%) of the leaf disc. In addition, one hundred percent (100%) of the FAW and CEW were killed after consuming the event MON 95379 containing leaf discs.

TABLE 5
Mean Leaf Damage Rating (LDR) scores and Mean Mortality
for R₀ plants expressing Cry1B.868 and Cry1Da_7.

| | FAW | CEW | SWCB |
|---|---|---|---|
| | Mean | Mean | Mean |
| Event | LDR | LDR | LDR |
| MON 95379 | 1.00 | 1.25 | 1.00 |
| Event 2 | 1.00 | 2.00 | 1.00 |
| Event 3 | 1.00 | 1.00 | 1.00 |
| Event 4 | 1.00 | 1.25 | 1.00 |
| Event 5 | 1.00 | 1.50 | 1.00 |
| Event 6 | 1.00 | 3.25 | 1.00 |
| Event 7 | 1.00 | 2.25 | 1.00 |
| Event 8 | 1.00 | 1.50 | 1.00 |
| Event 9 | 1.00 | 1.75 | 1.00 |
| Event 10 | 1.00 | 2.25 | 1.25 |
| Event 11 | 1.00 | 1.75 | 1.00 |
| Event 12 | 1.00 | 1.25 | 1.00 |
| Event 13 | 1.00 | 1.25 | 1.00 |
| Event 14 | 1.00 | 3.75 | 1.00 |
| Event 15 | 1.00 | 1.00 | 1.00 |
| Event 16 | 1.00 | 1.25 | 1.25 |
| Event 17 | 1.00 | 3.00 | 1.00 |
| Event 18 | 1.00 | 1.25 | 1.00 |
| Event 19 | 1.00 | 1.75 | 1.25 |
| Event 20 | 1.00 | 1.00 | 1.25 |
| Event 21 | 1.00 | 5.00 | 1.00 |
| Event 22 | 1.00 | 1.33 | 1.00 |
| Event 23 | 1.00 | 1.00 | 1.00 |
| Event 24 | 1.00 | 1.50 | 1.00 |
| Event 25 | 1.00 | 1.25 | 1.00 |
| Event 26 | 1.00 | 2.33 | 1.25 |
| Event 27 | 1.00 | 1.25 | 1.00 |
| Event 28 | 1.00 | 1.50 | 1.00 |
| Event 29 | 1.00 | 1.00 | 1.00 |
| Event 30 | 1.00 | 2.00 | 1.00 |
| Event 31 | 1.00 | 2.50 | 1.50 |
| Event 32 | 1.00 | 1.00 | 1.00 |
| Event 33 | 1.00 | 1.67 | 1.00 |
| Event 34 | 1.00 | 3.00 | 1.00 |
| Event 35 | 1.00 | 1.25 | 1.00 |
| Event 36 | 1.00 | 1.50 | 1.00 |
| Event 37 | 1.00 | 2.50 | 1.00 |
| Event 38 | 1.00 | 3.50 | 1.25 |
| Event 39 | 1.00 | 1.00 | 1.25 |
| Event 40 | 1.00 | 1.00 | 1.00 |
| Event 41 | 1.00 | 1.25 | 1.00 |

The forty-one (41) events were crossed with non-transgenic 93IDI3 variety plants. Ft heterozygous progeny plants were selected that comprised Construct MON95. Around five (5) F₁ plants for each event were artificially infested in a greenhouse for each insect pest species. With respect to FAW, approximately forty (40) neonates were used to infest each F₁ plant in the V6 to V8 stage whorl. With respect to SWCB, approximately thirty (30) neonates were used to infest the F₁ plant in the V6 to V10 stage whorl. Measures of leaf damage for FAW and SWCB were taken approximately fourteen (14) days after infestation. Tables 6 and 7 show the damage rating scales used to assess the leaf damage.

TABLE 6
Leaf damage rating scale for corn plants infested with FAW.

| Leaf Damage | |
|---|---|
| Rating (LDR) | Description |
| 0 | No visible damage |
| 1 | Only pinhole lesions present on whorl leaves |
| 2 | Pinholes and small, circular lesions present on whorl |
| | leaves |
| 3 | Small, circular lesions and 1-3 small, elongated lesions |
| | present on whorl and furl leaves |
| 4 | 4-6 small to mid-sized, elongated lesions present on |
| | 1-3 whorl and furl leaves |
| 5 | 4-6 large, elongated lesions present on 1-3 whorl and |
| | furl leaves and/or 1-3 small or mid-sized, uniform to |
| | irregular lesions, or both eaten from whorl and/or furl |
| | leaves |
| 6 | 4-6 large, elongated lesions present on 4-6 whorl and |
| | furl leaves and/or 4-6 large uniform to irregular shaped |
| | holes eaten from whorl and furl leaves |
| 7 | 7+ elongated lesions of all sizes present on several whorl |
| | and furl leaves plus 4-6 large uniform to irregular shaped |
| | holes eaten from the whorl and furl leaves |
| 8 | 7+ elongated lesions of all sizes on most whorl and furl |
| | leaves plus 7+ mid- to large-sized uniform to irregular- |
| | shaped holes eaten from the whorl and furl leaves |
| 9 | Whorl and furl leaves almost totally destroyed as well |
| | as the plant showing signs of stunting |

TABLE 7
Leaf damage rating scale for corn plants infested with SWCB.

| Leaf Damage | |
|---|---|
| Rating (LDR) | Description |
| 0 | No lesions |
| 1 | Small amount of pin or fine holes on leaves |
| 2 | Small amount of shot-hole injury on a few |
| | present |
| 3 | Shot-hole injury common on several leaves |
| 4 | Several leaves with shot-hole and elongated |
| | lesions |
| 5 | Several leaves with elongated lesions |
| 6 | Several leaves with elongated lesions-2.5 cm. |
| | Long |
| 7 | Long lesions common on ~50% of leaves |
| 8 | Long lesions common on ~70% of leaves |
| 9 | Most or all leaves with long lesions |

The SWCB infested F₁ plants were also assessed for the length of stalk boring caused by SWCB. To determine the length of stalk boring, corn stalks of the corn plants were broken at approximately eye level and the top portion was used to inspect for boring damage. The stalks were split using a double handled knife and the length of the tunnel bored out by SWCB was measured in centimeters (cm). In these experiments, the tunnel length was capped at ten centimeters (10 cm).

In addition, five (5) F₁ plants for each event were also infested with CEW to measure the amount of damage caused by CEW to the corn ear. Approximately forty (40) CEW nymphs were used to infest each plant and were placed on the green silks of R₁ stage plants. Twenty-one (21) days after infestation, the developing ears were examined, and the damage was recorded as cm² ear damage.

Table 8 shows the mean leaf damage ratings for the F₁ events infested with FAW and SWCB, the stalk boring lengths caused by SWCB, and the ear damage caused by CEW, wherein “NT” indicates not tested.

TABLE 8
Mean leaf damage ratings of F₁ transgenic
corn plants infested with FAW and SWCB, stalk boring
lengths caused by SWCB, and ear damage caused by CEW.

| | | | | CEW |
|---|---|---|---|---|
| | | | SWCB | ear |
| | | | tunnel | damage |
| | FAW | SWCB | length | (cm² |
| Event | LDR | LDR | (cm) | damage) |
| MON 95379 | 0.30 | 1.00 | 0.00 | 3.25 |
| Event 2 | 0.50 | 1.00 | 0.00 | 3.25 |
| Event 3 | 0.30 | 1.00 | 0.00 | 0.00 |
| Event 4 | 0.30 | 1.30 | 0.00 | 2.30 |
| Event 5 | 1.00 | 1.00 | 0.00 | 1.00 |
| Event 6 | 0.30 | 1.00 | 0.00 | 0.00 |
| Event 7 | 0.80 | 0.50 | 0.00 | 3.00 |
| Event 8 | 0.50 | 1.00 | 0.00 | 0.00 |
| Event 9 | 1.30 | 0.80 | 0.00 | 0.25 |
| Event 10 | NT | NT | NT | NT |
| Event 11 | NT | NT | NT | NT |
| Event 12 | 0.30 | 1.00 | 0.00 | 3.50 |
| Event 13 | NT | NT | NT | NT |
| Event 14 | NT | NT | NT | NT |
| Event 15 | NT | NT | NT | NT |
| Event 16 | NT | NT | NT | NT |
| Event 17 | 0.50 | NT | NT | NT |
| Event 18 | 0.00 | 1.30 | 0.00 | 2.50 |
| Event 19 | 0.80 | 1.30 | 0.00 | 0.00 |
| Event 20 | NT | NT | NT | NT |
| Event 21 | NT | NT | NT | NT |
| Event 22 | NT | NT | NT | NT |
| Event 23 | NT | NT | NT | NT |
| Event 24 | 0.50 | 0.50 | 2.50 | 2.75 |
| Event 25 | 0.00 | 1.50 | 2.25 | 0.00 |
| Event 26 | 0.30 | 1.30 | 0.00 | 3.00 |
| Event 27 | 0.50 | 1.50 | 2.50 | 1.25 |
| Event 28 | 0.30 | 1.00 | 1.00 | 1.75 |
| Event 29 | 0.30 | 1.00 | 1.00 | 0.25 |
| Event 30 | NT | NT | NT | NT |
| Event 31 | 0.30 | 1.00 | 0.00 | 2.50 |
| Event 32 | 0.50 | 1.00 | 0.00 | 0.25 |
| Event 33 | 0.50 | 1.00 | 0.00 | 2.25 |
| Event 34 | 0.30 | 1.00 | 0.00 | 1.50 |
| Event 35 | 0.30 | 1.30 | 1.25 | 0.00 |
| Event 36 | NT | NT | NT | NT |
| Event 37 | 0.50 | 1.00 | 0.00 | 1.50 |
| Event 38 | 1.30 | 1.00 | 0.00 | 0.50 |
| Event 39 | 0.00 | 1.00 | 1.75 | 2.00 |
| Event 40 | 0.30 | 0.50 | 1.25 | 1.75 |
| Event 41 | 0.00 | 0.80 | 0.00 | 2.25 |
| Negative Control | 7.80 | 9.00 | 10.00 | 13.25 |

As can be seen in Table 8, leaf damage to corn event MON 95379 was minimal for both FAW and SWCB when compared to the negative controls. Essentially, once the insects started to feed on the event MON 95379 F₁ leaf, expression of the Cry1B.868 and Cry1Da_7 insecticidal proteins in the corn leaves containing event MON 95379 caused the insect to cease consuming the leaf. SWCB tunneling was not observed in event MON 95379 while the negative controls showed extensive tunneling. With respect to CEW car damage, the damage to the ear was much less compared to the negative control, and was comparable to the ear damage observed in several commercially-available transgenic corn events. Infestation of the magnitude used in the F₁ assays was much higher than what is usually seen in nature. The F₁ assays demonstrated that corn event MON 95379 provides superior control of FAW, SWCB, and CEW.

In the summer of 2016, the F₁ progeny from the remaining thirty-nine (39) events after R₂/F₁ described in Example 2/Table 3 were assayed for resistance to FAW. CEW, and SWCB in field experiments using artificial infestation. Multiple locations were used to assay resistance.

FAW resistance was assayed in three (3) locations: Jerseyville. IL; Thomasboro, IL; and Union City, TN. In each location, each event was assayed in three (3) field plots using one (1) row per plot and thirty (30) seeds per row. Forty (40) FAW neonates were used to infest each plant twice, at the early and mid-whorl stage (V4 and V7 vegetative stage). Leaf feeding damage ratings were assessed using the scale as provided in Table 6.

SWCB resistance was assayed in three (3) locations: one (1) in Jonesboro. AR and two (2) in Union City, TN. In each location, each event was assayed in three (3) field plots using one (1) row per plot and thirty (30) seeds per row. Thirty (30) SWCB neonates were used to infest each plant at the mid-whorl stage (V7-V8). At the time of fifty percent (50%) pollen shed, the plants were infested again with thirty (30) SWCB neonates per plant. Stalk tunneling damage was assessed as previously described.

CEW resistance was assayed in five (5) locations: Jerseyville. IL. Jonesboro. AR. Monmouth, IL. Thomasboro. IL, and Union City, TN. In each location, each event was assayed in three (3) field plots using one (1) row per plot and thirty (30) seeds per row. Plants were infested when the silks were fresh and green, and some ear formation had started (R₁ through R₃ stage). CEW egg strips were used for infestation. Each strip contained approximately forty (40) eggs. One (1) strip was placed between the ear and stalk of each plant, with the eggs facing ear and close to the silks. Evaluation of ear damage was determined twenty-one (21) to twenty-eight (28) days after infestations. By this time, the insect has progressed from larval to pupal stage. Damage to the ears was measured as previously described.

For FAW and SWCB, data from all three (3) locations was used. For CEW, due to various field conditions, only data from Jonesboro, AR could be used. Table 9 shows the mean FAW leaf damage ratings, the SWCB tunnel lengths, and the CEW ear damage measurements for each of the tested events and the negative control.

TABLE 9
Mean FAW leaf damage ratings, SWCB tunnel length, and
CEW ear damage for 2016 field efficacy trials.

| | | SWCB | CEW |
|---|---|---|---|
| | | Tunnel | Ear |
| | FAW | Length | Damage |
| Event | LDR | (cm) | (cm2) |
| MON 95379 | 1.56 | 0.37 | 2.86 |
| Event 2 | 1.28 | 1.84 | 4.41 |
| Event 3 | 1.33 | 0.14 | 3.28 |
| Event 4 | 2.01 | 0.11 | 3.00 |
| Event 5 | 1.83 | 0.89 | 4.09 |
| Event 6 | 1.46 | 1.09 | 2.89 |
| Event 7 | 1.67 | 0.39 | 3.40 |
| Event 8 | 1.44 | 0.72 | 3.19 |
| Event 9 | 1.59 | 0.00 | 3.98 |
| Event 10 | 2.85 | 0.35 | 2.96 |
| Event 11 | 1.50 | 2.59 | 2.84 |
| Event 12 | 1.39 | 0.42 | 3.04 |
| Event 13 | 1.74 | 0.00 | 3.34 |
| Event 14 | 2.52 | 1.22 | 3.01 |
| Event 15 | 1.53 | 0.22 | 3.09 |
| Event 16 | 1.50 | 1.61 | 3.24 |
| Event 17 | 2.31 | 1.72 | 2.80 |
| Event 18 | 1.70 | 0.13 | 4.02 |
| Event 19 | 1.28 | 1.00 | 3.96 |
| Event 20 | 1.28 | 0.52 | 2.79 |
| Event 21 | 1.39 | 0.39 | 3.55 |
| Event 22 | 1.72 | 0.94 | 4.34 |
| Event 23 | 1.86 | 0.22 | 3.04 |
| Event 24 | 1.93 | 0.00 | 3.60 |
| Event 25 | 1.57 | 0.06 | 2.28 |
| Event 26 | 1.65 | 0.00 | 2.28 |
| Event 27 | 1.65 | 0.28 | 2.73 |
| Event 28 | 1.63 | 2.72 | 3.60 |
| Event 29 | 2.62 | 0.78 | 3.42 |
| Event 30 | 4.78 | 12.59 | 5.25 |
| Event 31 | 1.78 | 1.81 | 3.79 |
| Event 32 | 1.63 | 0.00 | 3.08 |
| Event 33 | 1.37 | 0.00 | 3.40 |
| Event 34 | 1.80 | 0.19 | 3.20 |
| Event 35 | 2.20 | 0.84 | 3.99 |
| Event 36 | 1.96 | 0.22 | 3.78 |
| Event 37 | 1.35 | 0.06 | 3.07 |
| Event 38 | 1.22 | 0.27 | 3.94 |
| Event 39 | 1.46 | 0.14 | 2.87 |
| Negative Control | 7.15 | 7.21 | 32.25 |

As demonstrated in Table 9, corn event MON 95379 provided excellent control of FAW, SWCB, and CEW when compared to the negative control. The level of infestation in these assays was much higher than what would normally be encountered in the field under natural conditions, demonstrating the superior performance of event MON 95379 under high insect pressure.

During concurrent field trials and Cre-excision of the selection cassette and the production of Gold Standard Seed, further characterization of the events was performed. As a result of extensive molecular characterization, efficacy, expression, and agronomic studies, events were dropped from testing, leaving two (2) events: Event 1 and MON 95379. Event 1 was de-prioritized based on observed yield drag in agronomic studies, leaving event MON 95379 for advancement.

During the 2016 to 2017 growing season in Argentina, event MON 95379 was assayed for resistance to FAW, CEW, and SCB in temperate and subtropical regions under natural infestation conditions. FAW leaf damage ratings were determined for event MON 95379 grown in the sub-tropical region of Argentina using the scale provided in Table 6. SCB tunneling data was obtained for event MON 95379 from two (2) locations in the temperate region of Argentina. CEW ear damage data was obtained for event MON 95379 from two (2) locations in the temperate region and three (3) locations in the subtropical regions of Argentina. Table 11 shows the mean FAW leaf damage ratings, SCB tunnel length, and CEW ear damage under natural infestation conditions for event MON 95379 and a negative control during the 2016-2017 Argentina growing seasons.

TABLE 10
Mean FAW leaf damage ratings, SCB tunnel length, and
CEW ear damage for 2016-2017 Argentina field efficacy trials.

| | | | CEW Ear |
|---|---|---|---|
| | FAW | SCB Tunnel | Damage |
| Event | (LDR) | Length (cm) | (cm2) |
| MON 95379 | 1.27 | 0.00 | 1.32 |
| Negative Control | 7.48 | 4.43 | 5.83 |

As can be seen in Table 10, event MON 95379 provided resistance to FAW. SCB, and CEW when compared to the negative control under natural infestation conditions in Argentina.

Event MON 95379 was also evaluated for resistance against FAW resistant to a commercially-available corn event (MON89034, which expresses Cry1a.105 and Cry2Ab2) over three (3) growing seasons in Puerto Rico (January 2016, July 2016, and January 2017). Table 11 shows the mean leaf damage ratings based upon the scale presented in Table 6 for each of the three (3) growing seasons compared with event MON89034 and the negative control.

TABLE 11
Mean leaf damage ratings for event MON 95379 and event
MON89034 naturally-infested with event MON89034-resistant FAW.

| | January | July | January |
|---|---|---|---|
| Event | 2016 | 2016 | 2017 |
| MON 95379 | 2.30 | 2.22 | 1.40 |
| MON89034 | 5.68 | 4.54 | 7.36 |
| Negative Control | 8.73 | 6.84 | 9.00 |

As can be seen in Table 11, corn event MON 95379 demonstrated resistance to event MON89034-resistant FAW under high natural pressure relative to the negative control.

In the summer of 2017, event MON 95379 was evaluated for resistance against FAW, SWCB, and CEW in the United States using methods similar to that described for the summer of 2016. FAW resistance was assayed at three (3) locations: Jerseyville. IL; Thomasboro, IL; and Monmouth, IL. In each location, each event was assayed in three (3) field plots using one (1) row per plot and thirty (30) seeds per row. Forty (40) FAW neonates were used to infest each plant two times. The first infestation occurred around V5 stage. The second infestation for plants in Monmouth. IL and Jerseyville. IL occurred around V8 stage. Due to a low hatch rate and poor weather, a second infestation was not possible in Thomasboro. IL. FAW leaf feeding damage ratings were assessed using the scale as provided in Table 6.

SWCB resistance was assayed at three (3) locations, one (1) in Jonesboro. AR and two (2) in Union City, IL. In each location, each event was assayed in three (3) field plots using one (1) row per plot and thirty (30) seeds per row. Thirty (30) SWCB neonates were used to infest each plant two times. Under normal conditions, the first infest is performed at the mid-whorl stage (V7-V8) in half of the row, but infestation was delayed about a week. Regardless, strong insect pressure was established. At the time of fifty percent (50%) pollen shed the second half of the row of plants were infested with thirty (30) SWCB neonates per plant. Stalk tunneling damage was assessed as previously described.

CEW resistance was assayed at six (6) locations: Jerseyville, IL, Jonesboro, AR, Paragould, AR. Monmouth, IL, and two locations in Union City. TN. In each location, each event was assayed in three (3) field plots using one (1) row per plot and thirty (30) seeds per row. Due to a shortage of insects, infestations in Monmouth, I L and Jerseyville, IL were infested two (2) to three (3) weeks later than when silks are fresh and green. In Monmouth, approximately twenty-two (22) neonates were used to infest each plant. In Jerseyville, IL, twenty-three (23) to twenty-four (24) neonates were used to infest partially opened corn ears. In Jonesboro. AR, one (1) of the three (3) rows received approximately thirty (30) neonates per plant, and the other two (2) rows received sixteen (16) to (18) neonates per plant. In Paragould, AR, all three (3) rows received approximately thirty (30) neonates per plant. Infestation was delayed in the two locations of Union City. TN due to insect availability. Both locations received eighteen (18) neonates per plant. Evaluation of ear damage was determined twenty-one (21) to twenty-eight (28) days after infestations. Damage to the ears was expressed as previously described. Artificial infestations were conducted on both marker and marker-free event MON 95379 plants. In addition, assays were also conducted using the natural insect pressure at the locations for the marker-containing event MON 95379 plants. Tables 12 and 13 show the FAW leaf damage ratings. SWCB tunnel lengths, and the CEW ear damage for marker-containing and marker-free event MON 95379 plants.

TABLE 12
Mean FAW leaf damage ratings, SWCB tunnel length, and CEW ear
damage for event MON 95379 plants before Cre-excision of the
selection marker under conditions of artificial and natural infestation.

| | Before Cre-excision of CP4 Marker | | | | |
|---|---|---|---|---|---|
| | | | | | SWCB |
| | | | | | Artificial |
| | FAW | FAW | CEW | CEW | Infestation |
| | Artificial | Natural | Artificial | Natural | Tunnel |
| | Infestation | Infestation | Infestation | Infestation | Length |
| Event | (LDR) | (LDR) | (cm2) | (cm2) | (cm) |
| MON | 1.17 | 1.15 | 4.93 | 5.81 | 0.00 |
| 95379 | | | | | |
| Negative | 7.08 | 8.15 | 8.49 | 14.57 | 14.34 |
| Control | | | | | |

TABLE 13
Mean FAW leaf damage ratings, SWCB tunnel length, and CEW
ear damage for marker-free event MON 95379 plants under
artificial infestation.

| | After Cre-excision of CP4 Marker | | |
|---|---|---|---|
| | | | SWCB |
| | FAW | CEW | Artificial |
| | Artificial | Artificial | Infestation |
| | Infestation | Infestation | Tunnel |
| Event | (EDR) | (cm2) | Length (cm) |
| MON | 1.20 | 5.39 | 0.27 |
| 95379 | | | |
| Negative | 7.08 | 8.49 | 14.34 |
| Control | | | |

As can be seen in Tables 12 and 13, event MON 95379 provided resistance against FAW, SWCB, and CEW under artificial (marker and marker-free) and natural (marker-free) infestation conditions.

In 2018, a hybrid cross of event MON 95379 with event MON89034 was assayed for resistance to FAW in a Brazil field trial under natural infestation conditions. The field trial was conducted in Santa Helena de Goias, State of Goids. In this location there are FAW populations resistant to the transgenic corn event MON89034. Transgenic corn plants corresponding to the cross of events MON 95379×MON89034, event MON89034, and a conventional corn plant (negative control) were planted. At V6 stage, leaf damage rating scores were determined for sixty (60) plants corresponding to the cross of events MON 95379×MON89034, thirty (30) plants corresponding to event MON89034, and thirty (30) negative controls using the scale presented in Table 6. In addition, the number of FAW neonates, larvae greater than two millimeters (2 mm) and less than or equal to 1.5 centimeters, and larvae greater than 1.5 centimeters were recorded for each plant. Table 14 shows the mean leaf damage ratings for the cross of events MON 95379×MON89034, event MON89034, and the negative control, along with the numbers of neonates and larvae observed on the corn plants.

TABLE 14
Mean FAW leaf damage rating and number of neonates and
larvae from Brazil, 2018 field trials for the cross of events MON
95379 × MON89034, event MON89034 and negative control.

| | FAW | | Larvae >2 mm | Larvae |
|---|---|---|---|---|
| Event | (LDR) | Neonates | and ≤1.5 cm | >1.5 cm |
| MON 95379 × | 0.62 | 0 | 0 | 0 |
| MON89034 | | | | |
| MON 89034 | 1.47 | 6 | 16 | 0 |
| Negative Control | 5.20 | 0 | 22 | 9 |

As can be seen in Table 14, the cross of events MON 95379×MON89034 provided resistance to FAW under natural infestation conditions relative to the negative control. The cross of events MON 95379×MON89034 also performed better than event MON89034 under conditions where event MON89034-resistant FAW are within the population of FAW. With respect to neonates and larvae, none were observed on the plants corresponding to the cross of events MON 95379×MON89034. Neonates and larvae between two (2) millimeters and one and a half (1.5) centimeters were observed on event MON89034 plants. The negative control plants were observed to have even more larvae than event MON89034 plants, and had larvae that had grown greater than 1.5 centimeters.

### Example 5

### Assay of Activity of Corn Event MON 95379 Against Lesser Cornstalk Borer

This Example describes the assay of activity of transgenic corn event MON 95379 against the Lepidopteran insect pest, Lesser Cornstalk Borer (LSCB. Elasmopalpus lignosellus).

Event MON 95379 was grown in a greenhouse along with negative control plants and infested with LSCB neonates. Ten (10) event MON 95379 plants and nine (9) negative control plants were grown in individual pots. Nine (9) days after planting, each plant was infested with ten (10) LSCB neonates per plant. Twenty-two (22) days after infestation, the plants were examined and rated for damage using a 0-4 damage rating scale as presented in Table 15.

TABLE 15
LSCB plant damage rating scale.

| LSCB | | |
|---|---|---|
| Damage | Severity of | |
| Rating | Injury | Description |
| 0 | No damage | Plants without injury |
| 1 | Slight injury | Plants with scratches in leaves and/or |
| | | stalk |
| 2 | Average damage | Plants with stalk parts damaged |
| 3 | Serious damage | Plants with stalk parts damage and |
| | | dead hear symptoms |
| 4 | Dead plant | Dead plants |

The resulting LSCB damage ratings for each plant is presented in Table 16.

TABLE 16
LSCB plant damage for each infested plant.

| Plant | MON 95379 | Negative Control |
|---|---|---|
| 1 | 0 | 4 |
| 2 | 0 | 2 |
| 3 | 0 | 3 |
| 4 | 0 | 3 |
| 5 | 0 | 4 |
| 6 | 0 | 3 |
| 7 | 0 | 3 |
| 8 | 0 | 4 |
| 9 | 0 | 4 |
| 10 | 0 | |

As can be seen in Table 16, LSCB produced extensive damage to the negative control plants, four (4) of which were rated as “Dead,” four (4) of which were rated as “Serious damage.” and only one (1) rated as “Average damage.” In contrast, the event MON 95379 LSCB infested plants showed no damage.

Transgenic corn event MON 95379 provides resistance to Lesser Cornstalk Borer (LSCB. Elasnopalpus lignosellus).

### Example 6

### Corn Event MON 95379 Provides Consistent Yield and Similar Agronomics to Untransformed LH244 Corn Plants

This Example demonstrates that transgenic corn event MON 95379 provides consistent yields and similar agronomics in the field to untransformed LH244 corn plants.

Field trials were conducted with plants corresponding to event MON 95379 prior to Cre-excision of the glyphosate selection cassette to determine various aspects of yield and agronomics in comparison to control plants. Measurements of yield were calculated and expressed as bushels per acre (bu/acre). Plant height and ear height were measured in inches (in). Fifty percent (50%) pollen shed and fifty percent (50%) silking were expressed as days after planting (DAP). Test weight, which is a measurement of bulk density, or the weight of a unit volume, of grain was expressed in pounds per bushel (lb/bu). The USDA established the standard test weight of a bushel of corn as fifty-six pounds per bushel (56 lb/bu) based upon a 15.5% moisture content. The percent moisture of the corn kernel was expressed on a wet weight basis. The moisture content is the amount of water in the seed and is usually expressed as a percentage. It can be expressed on either a wet weight basis (where it is expressed as a percentage of the fresh weight of the seed) or on a dry weight basis (where it is expressed as a percentage of the dry weight of the seed). Determination of percent moisture is destructive to the seed. Percent moisture (wet basis) can be calculated with the simple formula:

M_{wb}=(W_{w}/W_{w}+W_{d})×100

Where W_{w} is equal to the weight of the water and W_{d} is equal to the weight of the dry matter.

In the growing season of 2016 in the United States, yield and agronomic measures were determined for event MON 95379 inbreds and hybrids pre-Cre-excision of the glyphosate maker cassette. Tables 17 and 18 show the yield and agronomic characteristics measured for event MON 95379 inbreds and hybrids, respectively. The negative control plants for the inbred comparisons was untransformed variety LH244. Hybrids containing event MON 95379 were created by cross pollinating the inbred event MON 95379 with corn variety 93ID13, and the untransformed control was an LH244×93ID13 cross.

TABLE 17
Yield and agronomics for event MON 95379 inbreds
relative to non-transgenic controls.

| | | | Plant | | Ear | | 50% Pollen | | 50% | |
|---|---|---|---|---|---|---|---|---|---|---|
| | Yield | | Height | | Height | | Shed | | Silking | |
| | (bu/acre) | | (in) | | (in) | | (DAP) | | (DAP) | |
| Event | Mean | SE | Mean | SE | Mean | SE | Mean | SE | Mean | SE |
| MON 95379 | 111.08 | 5.17 | 85.40 | 2.72 | 41.29 | 1.45 | 65.11 | 2.00 | 65.77 | 2.05 |
| LH244 | 114.18 | 5.02 | 82.05 | 2.32 | 38.73 | 1.33 | 62.60 | 1.79 | 63.54 | 1.82 |

TABLE 18
Yield and agronomic for event MON 95379 hybrids
relative to non-transgenic controls.

| | Yield | | Test Weight | | Percent | |
|---|---|---|---|---|---|---|
| | (bu/acre) | | (lb/bu) | | Moisture | |
| Event | Mean | SE | Mean | SE | Mean | SE |
| MON 95379 × 93IDI3 | 203.48 | 7.04 | 58.78 | 0.61 | 16.04 | 0.42 |
| LH244 × 93IDI3 | 196.45 | 6.81 | 58.31 | 0.61 | 15.89 | 0.43 |

As can be seen in Tables 17 and 18, the yield and other agronomic measures for event MON 95379 in the 2016 United States field trials were relatively the same for both inbreds and hybrids relative to the controls. The variability between the inbreds and hybrids and their respective controls was within acceptable limits and demonstrate there were no negative impacts on yield and other agronomic characteristics caused by insertion of the T-DNA into the corn genome of event MON 95379.

Yield and agronomics were also studied in Argentina during the 2016 to 2017 growing season for event MON 95379 inbreds and hybrids pre-Cre-excision of the glyphosate marker cassette. Tables 19 and 20 show the yield and agronomic characteristics measured for event MON 95379 inbreds. The negative control plants for the inbred comparisons was untransformed variety LH244. Hybrids containing event MON 95379 were created by cross-pollinating events MON89034×MON895379. The transgenic control was event MON88017×event MON89034. The non-transgenic control was a LH244×931DI3 cross. Table 21 shows the yield and agronomic characteristics measured for event MON 95379 hybrids, wherein “NC” indicates not calculated.

TABLE 19
Yield and agronomics for event MON 95379 inbreds
relative to non-transgenic controls.

| | | | Plant | | Ear | | 50% Pollen | | 50% | |
|---|---|---|---|---|---|---|---|---|---|---|
| | Yield | | Height | | Height | | Shed | | Silking | |
| | (bu/acre) | | (in) | | (in) | | (DAP) | | (DAP) | |
| Event | Mean | SE | Mean | SE | Mean | SE | Mean | SE | Mean | SE |
| MON 95379 | 113.39 | 4.57 | 73.89 | 1.05 | 38.38 | 0.95 | 64.32 | 0.60 | 64.68 | 0.62 |
| LH244 | 105.49 | NC | 71.69 | NC | 36.66 | NC | 64.86 | NC | 65.17 | NC |

TABLE 20
Yield and agronomics for event MON 95379 inbreds relative
to non-transgenic controls.

| | Test Weight | | | |
|---|---|---|---|---|
| | (lb/bu) | | Percent Moisture | |
| Event | Mean | SE | Mean | SE |
| MON 95379 | 59.28 | 0.10 | 16.54 | 0.26 |
| LH244 | 59.32 | NC | 16.01 | NC |

TABLE 21
Yield and agronomics for event MON 95379 hybrids relative to
transgenic and non-transgenic controls.

| | Yield | | Test Weight | | Percent | |
|---|---|---|---|---|---|---|
| | (bu/acre) | | (lb/bu) | | Moisture | |
| Event | Mean | SE | Mean | SE | Mean | SE |
| MON89034 × MON 95379 | 165.80 | 5.94 | 60.06 | 0.20 | 15.12 | 0.18 |
| MON88017 × MON89034 | 164.87 | 5.94 | 59.62 | 0.20 | 15.45 | 0.18 |
| LH244 × 93IDI3 | 166.82 | NC | 59.70 | NC | 15.38 | NC |

As can be seen in Tables 19 through 21, the measures of yield and other agronomic characteristics were relatively the same for event MON 95379 inbreds and hybrids relative to the controls.

In 2017, yield and agronomics were again measured in field trials in the United States for event MON 95379 inbreds and hybrids pre-Cre-excision of the glyphosate marker cassette. Inbred and hybrid controls were similar to those used in the 2016 United States field trials. Table 22 shows the yield and agronomic characteristics for event MON 95379 inbreds relative to non-transgenic controls, and Tables 23 and 24 show the yield and agronomic characteristics measured for event MON 95379 hybrids relative to non-transgenic controls in the 2017 United States field trials.

TABLE 22
Yield and agronomics for event MON 95379
inbreds relative to non-transgenic controls.

| | Yield | | Test Weight | | Percent | |
|---|---|---|---|---|---|---|
| | (bu/acre) | | (lb/bu) | | Moisture | |
| Event | Mean | SE | Mean | SE | Mean | SE |
| MON 95379 | 116.49 | 5.45 | 57.86 | 0.59 | 21.16 | 0.76 |
| LH244 | 124.46 | 5.39 | 58.60 | 0.58 | 20.57 | 0.76 |

TABLE 23
Yield and agronomic for event MON 95379 hybrids
relative to non-transgenic controls.

| | | | | | | | 50% | | | |
|---|---|---|---|---|---|---|---|---|---|---|
| | | | Plant | | Ear | | Pollen | | 50% | |
| | Yield | | Height | | Height | | Shed | | Silking | |
| | (bu/acre) | | (in) | | (in) | | (DAP) | | (DAP) | |
| Event | Mean | SE | Mean | SE | Mean | SE | Mean | SE | Mean | SE |
| MON 95379 × 93IDI3 | 213.15 | 4.84 | 99.14 | 2.50 | 45.27 | 1.77 | 56.65 | 0.93 | 57.17 | 1.06 |
| LH244 × 93IDI3 | 217.86 | 4.44 | 97.42 | 2.28 | 45.61 | 1.45 | 55.87 | 0.91 | 56.47 | 1.04 |

TABLE 24
Yield and agronomic for event MON 95379 hybrids relative
to non-transgenic controls.

| | Test Weight | | | |
|---|---|---|---|---|
| | (lb/bu) | | Percent Moisture | |
| Event | Mean | SE | Mean | SE |
| MON 95379 × 93IDI3 | 57.98 | 0.37 | 19.73 | 0.51 |
| LH244 × 93IDI3 | 57.53 | 0.36 | 19.74 | 0.50 |

As can be seen in Tables 22 through 24, the yield and other agronomic properties event MON 95379 demonstrated in the 2017 United Stated field trials were similar to the untransformed controls for both inbred and hybrid lines.

During the 2018 to 2019 growing season in Argentina, agronomics and yield were measured in field trials for event MON 95379 inbreds and hybrids post-Cre-excision of the glyphosate marker cassette. Inbred controls were similar to those used in the 2017 United States field trials. The hybrids were produced through crosses with the elite variety 80IDM2. Tables 25 and 26 show the yield and agronomic characteristics for event MON 95379 inbreds relative to non-transgenic controls, and Table 27 shows the yield and agronomic characteristics measured for event MON 95379 hybrids relative to non-transgenic controls in the 2018 to 2019 Argentina field trials.

TABLE 25
Yield and agronomics for event MON 95379 inbreds
relative to non-transgenic controls.

| | | | | | | | 50% | | | |
|---|---|---|---|---|---|---|---|---|---|---|
| | | | Plant | | Ear | | Pollen | | 50% | |
| | Yield | | Height | | Height | | Shed | | Silking | |
| | (bu/acre) | | (in) | | (in) | | (DAP) | | (DAP) | |
| Event | Mean | SE | Mean | SE | Mean | SE | Mean | SE | Mean | SE |
| MON 95379 | 92.81 | 7.30 | 85.14 | 2.45 | 40.57 | 2.06 | 62.74 | 0.61 | 63.77 | 0.65 |
| LH244 | 103.46 | 7.68 | 83.93 | 1.72 | 38.57 | 1.24 | 62.93 | 0.61 | 63.88 | 0.71 |

TABLE 26
Yield and agronomics for event MON 95379 inbreds relative
to non-transgenic controls.

| | Test Weight | | | |
|---|---|---|---|---|
| | (lb/bu) | | Percent Moisture | |
| Event | Mean | SE | Mean | SE |
| MON | 59.59 | 1.52 | 17.49 | 0.96 |
| 95379 | | | | |
| LH244 | 60.34 | 0.97 | 17.24 | 0.69 |

TABLE 27
Yield and agronomics for event MON 95379 hybrids
relative to non-transgenic controls.

| | | | Test | | | |
|---|---|---|---|---|---|---|
| | Yield | | Weight | | Percent | |
| | (bu/acre) | | (lb/bu) | | Moisture | |
| Event | Mean | SE | Mean | SE | Mean | SE |
| MON 95379 × | 206.86 | 8.37 | 59.42 | 0.46 | 19.03 | 0.84 |
| 80IDM2 | | | | | | |
| LH244 x 80IDM2 | 207.81 | 8.11 | 59.22 | 0.45 | 19.12 | 0.83 |

As can be seen in Tables 25 through 27, the yield and other agronomic properties event MON 95379 demonstrated in the 2017 to 2018 Argentina field trials were similar to the untransformed controls for both inbred and hybrid lines.

Thus, in sum, corn event MON 95379 demonstrated similar yield and other agronomic properties over four (4) separate growing seasons in the United States and Argentina. Event MON 95379 does not negatively affect yield or cause a change in other agronomic properties measured compared to non-transgenic and transgenic controls.

### Example 7

### Corn Event MON 95379 Event-Specific Endpoint TAQMAN® Assays

The following Example describes methods useful in identifying the presence of event MON 95379 in a corn sample. A pair of PCR primers and a probe were designed for the purpose of identifying the unique junction formed between the corn genomic DNA and the inserted DNA of event MON 95379 in an event-specific endpoint TAQMAN® PCR. Examples of conditions utilized for identifying the presence of event MON 95379 in a corn sample in an event-specific endpoint TAQMAN® PCR are described in Table 28 and Table 29.

The sequence of the oligonucleotide forward primer SQ21529 (SEQ ID NO:15) is identical to the nucleotide sequence corresponding to positions 833-852 of SEQ ID NO:10. The sequence of the oligonucleotide reverse primer SQ21524 (SEQ ID NO:16) is identical to the reverse complement of the nucleotide sequence corresponding to positions 905-934 of SEQ ID NO:10. The sequence of the oligonucleotide probe PB10269 (SEQ ID NO:17) is identical to the reverse complement of the nucleotide sequence corresponding to positions 886-901 of SEQ ID NO:10. The primers SQ21529 (SEQ ID NO:15) and SQ21524 (SEQ ID NO:16) with probe PB10269 (SEQ ID NO:17), which may be fluorescently labeled (e.g., a 6-FAM™ fluorescent label), can be used in an endpoint TAQMAN® PCR assay to identify the presence of DNA derived from event MON 95379 in a sample.

In addition to SQ21529 (SEQ ID NO:15). SQ21524 (SEQ ID NO:16), and PB10269 (SEQ ID NO:17), it should be apparent to persons skilled in the art that other primers and/or probes can be designed to either amplify or hybridize to sequences within SEQ ID NO:10 which are unique to, and useful for, detecting the presence of DNA derived from event MON 95379 in a sample.

Following standard molecular biology laboratory practices, PCR assays for event identification were developed for detection of event MON 95379 in a sample. Parameters of either a standard PCR assay or a TAQMAN® PCR assay were optimized with each set of primer pairs and probes (e.g., probes labeled with a fluorescent tag such as 6-FAM™) used to detect the presence of DNA derived from event MON 95379 in a sample. A control for the PCR reaction includes internal control primers and an internal control probe (e.g., VIC®-labeled) specific to a region within the corn genome that is used as an internal control, and are primers SQ20222 (SEQ ID NO:18), SQ20221 (SEQ ID NO:19), and VIC® labeled probe PB50237 (SEQ ID NO:20).

Generally, the parameters which were optimized for detection of event MON 95379 in a sample included primer and probe concentration, amount of templated DNA, and PCR amplification cycling parameters. The controls for this analysis include a positive control from corn containing event MON 95379, a negative control from non-transgenic corn, and a negative control that contains no template DNA.

TABLE 28
MON 95379 event-specific endpoint TAQMAN ® PCR
reaction components.

| | | Stock | | Final | |
|---|---|---|---|---|---|
| | | Concen- | | Concen- | |
| | | tration | Volume | tration | |
| Step | Reagent | (μM) | (μl) | (μM) | Comments |
| | Reaction volume | | 5 | | |
| 1 | 2× Master Mix | | 2.28 | | 1× final |
| | | | | | concentration |
| 2 | Event Specific | 100 | 0.05 | 0.9 | |
| | Primer SQ51219 | | | | |
| 3 | Event Specific | 100 | 0.05 | 0.9 | |
| | Primer SQ21524 | | | | |
| 4 | Event Specific | 100 | 0.01 | 0.2 | Probe is |
| | 6FAM ™ probe | | | | light |
| | PB10269 | | | | sensitive |
| 5 | Internal Control | 100 | 0.05 | 0.9 | |
| | Primer SQ20222 | | | | |
| 6 | Internal Control | 100 | 0.05 | 0.9 | |
| | Primer SQ20221 | | | | |
| 7 | Internal Control | 100 | 0.01 | 0.2 | Probe is |
| | VIC ® probe | | | | light |
| | PB50237 | | | | sensitive |
| 8 | Extracted DNA | | 2.5 | | Separate |
| | (template): | | | | reactions |
| | Leaf Samples to | | | | are made |
| | be analyzed | | | | for each |
| | Negative control | | | | template. |
| | (non-transgenic | | | | |
| | DNA) | | | | |
| | Negative water | | | | |
| | control (No | | | | |
| | template control) | | | | |
| | Positive Qualitative | | | | |
| | control(s) | | | | |
| | MON 95379 | | | | |
| | DNA | | | | |

TABLE 29
Endpoint TAQMAN ® thermocycler conditions.

| Step | Number | |
|---|---|---|
| No. | of Cycles | Settings |
| 1 | 1 | 95° C. 20 seconds |
| 2 | 40 | 95° C. 3 seconds |
| | | 60° C. 20 seconds |
| 3 | 1 | 10° C. |

### Example 8

### Assays for Determining Zygosity for Event MON 95379 Using TAQMAN® and Detection of the Insect Toxin Transgenes

The following Example describes methods useful in identifying the zygosity of event MON 95379 and detection of the insect toxin transgenes in event MON 95379 in a corn sample. Pairs of PCR primers and a probe are designed for the purpose of identifying specific properties of alleles positive and negative for the T-DNA insertion that gave rise to event MON 95379.

A zygosity assay is useful for determining if a plant comprising an event is homozygous for the event DNA (i.e., comprising the exogenous DNA in the same location on each chromosome of the chromosomal pair), heterozygous for the event DNA (i.e., comprising the exogenous DNA on only one chromosome of the chromosomal pair), or wild-type (i.e., null for the event DNA).

An endpoint TAQMAN® thermal amplification method was used to develop a zygosity assay for event MON 95379. The assay uses a primer pair and a probe to detect amplicons corresponding to one of the two insect toxin coding sequences encoding Cry1B.868 and Cry1 Da_7 comprised within the T-DNA used to generate corn event MON 95379. In addition, a primer pair and probe are used to detect a single-copy internal control that is located within the corn genome and is known to be present as a homozygous allele.

For this assay two (2) primer pairs and two (2) probes were mixed together with the sample. The DNA primers used in the zygosity assay which detects the presence of the Cry1B.868 toxin coding sequence were primers SQ50998 (SEQ ID NO:21) and SQ50997 (SEQ ID NO:22). The VIC®-labeled DNA probe used in the zygosity assay which detects the presence of the Cry1B.868 toxin coding sequence was PB54340 (SEQ ID NO:23). The DNA primers used in the zygosity assay which detect the presence of the Cry1Da_7 toxin coding sequence were primers SQ50485 (SEQ ID NO:24) and SQ50484 (SEQ ID NO:25). The VIC®-labeled DNA probe used in the zygosity assay which detects the presence of the Cry1Da_7 toxin coding sequence was PB50138 (SEQ ID NO:26). Both zygosity detection assays use the same internal control. The primers for the internal control were SQ20222 (SEQ ID NO: 18) and SQ20221 (SEQ ID NO: 19), and the 6FAM™-labeled probe for the internal control was PB50237 (SEQ ID NO:20). The DNA primers and probe for either Cry1B.868 or Cry1 Da_7 were mixed with the primers and probe for the internal control as shown in Tables 30 and 31.

TABLE 30
Corn event MON 95379 zygosity TAQMAN ® PCR for the
detection of Cry1B.868.

| | | Stock | | Final | |
|---|---|---|---|---|---|
| | | Concen- | | Concen- | |
| | | tration | Volume | tration | |
| Step | Reagent | (μl) | (μl) | (μM) | Comments |
| | Reaction volume | | 5 | | |
| 1 | 2× Master Mix | | 2.4 | | 1× final |
| | | | | | concentration |
| 2 | Cry1B.868 specific | 100 | 0.0225 | 0.45 | |
| | primer SQ50998 | | | | |
| 3 | Cry1B.868 specific | 100 | 0.0225 | 0.45 | |
| | primer SQ50997 | | | | |
| 4 | Cry1B.868 | 100 | 0.005 | 0.1 | Probe is |
| | 6FAM ™ probe | | | | light |
| | PB50340 | | | | sensitive |
| 5 | Internal Control | 100 | 0.0225 | 0.45 | |
| | Primer SQ20222 | | | | |
| 6 | Internal Control | 100 | 0.0225 | 0.45 | |
| | Primer SQ20221 | | | | |
| 7 | Internal Control | 100 | 0.005 | 0.1 | Probe is |
| | VIC ® probe | | | | light |
| | PB50237 | | | | sensitive |
| 8 | Extracted DNA | | 2.5 | | Separate |
| | (template): | | | | reactions |
| | Leaf Samples to | | | | are made |
| | be analyzed | | | | for each |
| | Negative control | | | | template. |
| | (non-transgenic | | | | |
| | DNA) | | | | |
| | Negative water | | | | |
| | control (No | | | | |
| | template control) | | | | |
| | Positive copy | | | | |
| | number control(s) | | | | |
| | Cry1B.868 | | | | |

TABLE 31
Corn event MON 95379 zygosity TAQMAN ® PCR for the
detection of Cry1Da_7.

| | | Stock | | Final | |
|---|---|---|---|---|---|
| | | Concen- | | Concen- | |
| | | tration | Volume | tration | |
| Step | Reagent | (μl) | (μl) | (μM) | Comments |
| | Reaction volume | | 5 | | |
| 1 | 2× Master Mix | | 2.4 | | 1× final |
| | | | | | concentration |
| 2 | Cry1Da_7 specific | 100 | 0.0225 | 0.45 | |
| | Primer SQ50485 | | | | |
| 3 | Cry1Da_7 specific | 100 | 0.0225 | 0.45 | |
| | Primer SQ50484 | | | | |
| 4 | Cry1Da_7 specific | 100 | 0.005 | 0.1 | Probe is |
| | 6FAM ™ probe | | | | light |
| | PB50138 | | | | sensitive |
| 5 | Internal Control | 100 | 0.0225 | 0.45 | |
| | Primer SQ20222 | | | | |
| 6 | Internal Control | 100 | 0.0225 | 0.45 | |
| | Primer SQ20221 | | | | |
| 7 | Internal Control | 100 | 0.005 | 0.1 | Probe is |
| | VIC ® probe | | | | light |
| | PB50138 | | | | sensitive |
| 8 | Extracted DNA | | 2.5 | | Separate |
| | (template): | | | | reactions |
| | Leaf Samples to | | | | are made |
| | be analyzed | | | | for each |
| | Negative control | | | | template. |
| | (non-transgenic | | | | |
| | DNA) | | | | |
| | Negative water | | | | |
| | control (No | | | | |
| | template control) | | | | |
| | Positive copy | | | | |
| | number | | | | |
| | control(s) | | | | |
| | Cry1Da_7 | | | | |

Separate reactions are mixed using DNA derived from a leaf sample for which zygosity is not known, a negative control of DNA derived from an untransformed corn plant, a negative control lacking DNA, and a positive control using DNA derived from a transgenic plant homozygous for Cry11B.868 or Cry11 Da-7, depending upon which toxin coding sequence is used for detection. The reactions are then subjected to the thermal cycles presented in Table 32.

TABLE 32
Zygosity TAQMAN ® Thermocycler conditions.

| Step | Number | |
|---|---|---|
| No. | of Cycles | Settings |
| 1 | 1 | 95° C. 20 seconds |
| 2 | 40 | 95° C. 3 seconds |
| | | 60° C. 20 seconds |
| 3 | 1 | 10° C. |

After amplification, the cycle thresholds (Ct values) were determined for the amplicon corresponding to the toxin coding sequence and the single-copy, homozygous internal standard. The difference (ΔCt) between the Ct value of the single-copy, homozygous internal standard amplicon and the Ct value of the toxin coding sequence amplicon was determined. With respect to zygosity, a ΔCt of around zero (0) indicated homozygosity of the inserted event MON 95379 T-DNA and ΔCt of around one (1) indicated heterozygosity of the inserted event MON 95379 T-DNA. Lack of an amplicon corresponding to the insect toxin coding sequence indicated the sample is null for the inserted event MON 95379 T-DNA. The Ct values in the TAQMAN® thermal amplification method will have some variability due to multiple factors such as amplification efficiency and ideal annealing temperatures. Therefore, the range of “about one (1)” is defined as a ΔCt of 0.75 to 1.25.

For each progeny derived from a cross with event MON 95379, assays were performed for both toxin coding sequences to assure accuracy in the determination of zygosity of the progeny.

### Example 9

### Assays for Determining Zygosity for Corn Event MON 95379 Using TAQMAN®

The following Example describes a method useful in identifying the zygosity of event MON 95379 in a corn sample.

Pairs of PCR primers and a probe are designed for the purpose of identifying specific properties of alleles positive and negative for the T-DNA insertion that gave rise to event MON 95379. Examples of conditions that may be used in an event-specific zygosity TAQMAN® PCR are provide in Tables 33 and 34. For this assay, three primers and two probes were mixed together with the sample. The DNA primers used in the zygosity assay were primers SQ50219 (SEQ ID NO:15). SQ21524 (SEQ ID NO:16), and PWTDNA (SEQ ID NO:27). The probes used in the zygosity assay were 6FAM™-labeled probe PB10269 (SEQ ID NO:17) and VIC®-labeled probe PRWTDNA (SEQ ID NO:28). Primers SQ50219 (SEQ ID NO:15) and SQ21524 (SEQ ID NO:16) and the 6FAM™-labeled probe PB10269 (SEQ ID NO:17) are diagnostic for event MON 95379 DNA. SQ50219 (SEQ ID NO:15) and PWTDNA (SEQ ID NO:27) and the VIC®-labeled probe PRWTDNA (SEQ ID NO:28) are diagnostic when there is no copy of event MON 95379; i.e., they are diagnostic for the wild type allele.

When the three primers and two probes are mixed together in a PCR reaction with DNA extracted from a plant heterozygous for event MON 95379, there is a fluorescent signal from both the 6FAM™-labeled probe PB10269 (SEQ ID NO:17) and the VIC®-labeled probe PRWTDNA (SEQ ID NO:28) which is indicative of and diagnostic for a plant heterozygous for event MON 95379. When the three primers and two probes are mixed together in a PCR reaction with DNA extracted from a plant homozygous for event MON 95379, there is a fluorescent signal from only the 6FAM™-labeled probe PB10269 (SEQ ID NO:17) and not the VIC®-labeled probe PRWTDNA (SEQ ID NO:28). When the three primers and the two probes are mixed together in a PCR reaction with DNA extracted from a plant which is null for event MON 95379 (i.e., the wild-type), there is a fluorescent signal from only the VIC®-labeled probe PRWTDNA (SEQ ID NO:28). The template DNA samples and controls for this analysis were a positive control from corn containing event MON 95379 DNA (from both a known homozygous and a known heterozygous sample), a negative control from non-transgenic corn and a negative control that contains no template DNA.

TABLE 33
Event MON 95379 zygosity TAQMAN ® PCR

| | | Stock | | Final | |
|---|---|---|---|---|---|
| | | Concen- | | Concen- | |
| | | tration | Volume | tration | |
| Step | Reagent | (μl) | (μl) | (μM) | Comments |
| | Reaction volume | | 5 | | |
| 1 | 18 megohm water | | 0.33 | | Adjust for |
| | | | | | final volume |
| 2 | 2× Master Mix | | 2.5 | | 1× final |
| | | | | | concentration |
| 3 | Event Specific | 100 | 0.05 | 0.9 | |
| | Primer SQ51219 | | | | |
| 4 | Event Specific | 100 | 0.05 | 0.9 | |
| | Primer SQ21524 | | | | |
| 5 | Event Specific | 100 | 0.01 | 0.2 | Probe is |
| | 6FAM ™ probe | | | | light |
| | PB10269 | | | | sensitive |
| 6 | WT allele Primer | 100 | 0.05 | 0.9 | |
| | PNEGDNA | | | | |
| 7 | WT allele | 100 | 0.01 | 0.2 | Probe is |
| | VIC ® probe | | | | light |
| | PRBNEGDNA | | | | sensitive |
| 8 | Extracted DNA | | 2.5 | | Separate |
| | (template): | | | | reactions |
| | Leaf Samples to | | | | are made |
| | be analyzed | | | | for each |
| | Negative control | | | | template. |
| | (non-transgenic | | | | |
| | DNA) | | | | |
| | Negative water | | | | |
| | control (No | | | | |
| | template control) | | | | |
| | Positive Qualitative | | | | |
| | control(s) | | | | |
| | MON 95379 | | | | |
| | DNA | | | | |

TABLE 34
Zygosity TAQMAN ® thermocycler conditions

| Step | Number | |
|---|---|---|
| No. | of Cycles | Settings |
| 1 | 1 | 95° C. 20 seconds |
| 2 | 40 | 95° C. 3 seconds |
| | | 60° C. 20 seconds |
| 3 | 1 | 10° C. |

### Example 10

### Identification of Corn Event MON 95379 in any MON 95379 Breeding Event

The following Example describes how one may identify the MON 95379 event within progeny of any breeding activity using corn event MON 95379.

DNA primer pairs are used to produce an amplicon diagnostic for corn event MON 95379. An amplicon diagnostic for event MON 95379 comprises at least one junction sequence. The junction sequences for event MON 95379 are SEQ ID NO:1, SEQ ID NO:2, SEQ ID NO:3, SEQ ID NO:4. SEQ ID NO:5. SEQ ID NO:6. SEQ ID NO:7, and SEQ ID NO:8 ([1], [2], [3], [4], [5], [6], [7], and [8], respectively in FIG. 1). SEQ ID NO:1 is a fifty (50) nucleotide sequence representing the 5′ junction region of corn genomic DNA and the integrated transgenic expression cassette. SEQ ID NO:1 is positioned in SEQ ID NO:10 at nucleotide position 838-887. SEQ ID NO:2 is a fifty (50) nucleotide sequence representing the 3′ junction region of corn genomic DNA and the integrated transgenic expression cassette. SEQ ID NO:2 is positioned in SEQ ID NO:10 at nucleotide position 14156-14205. SEQ ID NO:3 is a one hundred (100) nucleotide sequence representing the 5′ junction region of corn genomic DNA and the integrated transgenic expression cassette. SEQ ID NO:3 is positioned in SEQ ID NO:10 at nucleotide position 813-912. SEQ ID NO:4 is a one hundred (100) nucleotide sequence representing the 3′ junction region of corn genomic DNA and the integrated transgenic expression cassette. SEQ ID NO:4 is positioned in SEQ ID NO:10 at nucleotide position 14,131-14,230. SEQ ID NO:5 is a two hundred (200) nucleotide sequence representing the 5′ junction region of corn genomic DNA and the integrated transgenic expression cassette. SEQ ID NO:5 is positioned in SEQ ID NO:10 at nucleotide position 763-962. SEQ ID NO:6 is a two hundred (200) nucleotide sequence representing the 3′ junction region of corn genomic DNA and the integrated transgenic expression cassette. SEQ ID NO:6 is positioned in SEQ ID NO:10 at nucleotide position 14,081-14.280. SEQ ID NO:7 is a one thousand one hundred sixty (1.160) nucleotide sequence representing the 5′ junction region of corn genomic DNA and the integrated transgenic expression cassette. SEQ ID NO:7 is positioned in SEQ ID NO:10 at nucleotide positions 1-1.160. SEQ ID NO:8 is a one thousand one hundred seventy eight (1,178) nucleotide sequence representing the 3′ junction region of the integrated transgenic expression cassette and the corn genomic DNA. SEQ ID NO:8 is positioned in SEQ ID NO:10 at nucleotide positions 14,039-15.216.

Primer pairs that will produce an amplicon diagnostic for event MON 95379 include primer pairs based upon the flanking sequences (SEQ ID NO:11 and SEQ ID NO:12) and the inserted T-DNA (SEQ ID NO:9). To acquire a diagnostic amplicon in which SEQ ID NO:1, or SEQ ID NO:3, or SEQ ID NO:5, or SEQ ID NO:7 is found, one would design a forward primer molecule based upon the 5′ flanking corn genomic DNA (SEQ ID NO:11; from bases 1 to 862 of SEQ ID NO:10) and a reverse primer molecule based upon the inserted T-DNA (SEQ ID NO:9; from positions 863 through 14.180 of SEQ ID NO:10) in which the primer molecules are of sufficient length of contiguous nucleotides to specifically hybridize to SEQ ID NO:1 1 and SEQ ID NO:9. To acquire a diagnostic amplicon in which SEQ ID NO:2, or SEQ ID NO:4, or SEQ ID NO:6, or SEQ ID NO:8 is found, one would design a forward primer molecule based upon the inserted T-DNA (SEQ ID NO:9; from positions 863 through 14,180 of SEQ ID NO:10) and a reverse primer molecule based upon the 3′ flanking corn genomic DNA (SEQ ID NO:12; from positions 14,181 through 15,216 of SEQ ID NO:10) in which the primer molecules are of sufficient length of contiguous nucleotides to specifically hybridize to SEQ ID NO:9 and SEQ ID NO:12.

For practical purposes, one should design primers which produce amplicons of a limited size range, preferably between 200 to 1000 bases. Smaller sized amplicons in general are more reliably produced in PCR reactions, allow for shorter cycle times, and can be easily separated and visualized on agarose gels or adapted for use in endpoint TAQMAN®-like assays. In addition, amplicons produced using said primer pairs can be cloned into vectors, propagated, isolated and sequenced, or can be sequenced directly with methods well established in the art. Any primer pair derived from the combinations of SEQ ID NO:11 and SEQ ID NO:9 or SEQ ID NO:12 and SEQ ID NO:9 that are useful in a DNA amplification method to produce an amplicon diagnostic for event MON 95379 or progeny thereof is an aspect of the present invention. Any single isolated DNA polynucleotide primer molecule comprising at least eleven (11) contiguous nucleotides of SEQ ID NO:11. SEQ ID NO:9 or SEQ ID NO:12 or their complements that is useful in a DNA amplification method to produce an amplicon diagnostic for event MON 95379 or progeny thereof is an aspect of the present invention.

An example of the amplification conditions for this analysis is illustrated in Tables 28 and 29. Any modification of these methods or the use of DNA primers homologous or complementary to SEQ ID NO:11 or SEQ ID NO:12, or DNA sequences of the genetic elements contained in the transgene insert (SEQ ID NO:9) of event MON 95379, that produce an amplicon diagnostic for event MON 95379 is within the art. A diagnostic amplicon comprises a DNA molecule homologous or complementary to at least one transgene/genomic junction DNA or a substantial portion thereof.

An analysis for an event MON 95379 containing plant tissue sample should include a positive tissue control from a plant that contains event MON 95379, a negative control from a corn plant that does not contain event MON 95379 (e.g., LH244), and a negative control that contains no corn genomic DNA. A primer pair will amplify an endogenous corn DNA molecule and will serve as an internal control for the DNA amplification conditions. Additional primer sequences can be selected from SEQ ID NO: 11, SEQ ID NO: 12, or SEQ ID NO: 9 by those skilled in the art of DNA amplification methods. Conditions selected for the production of an amplicon by the methods shown in Table 28 and Table 29 may differ, but result in an amplicon diagnostic for event MON 95379 DNA. The use of DNA primer sequences within or with modifications to the methods of Table 28 and Table 29 are within the scope of the invention. An amplicon produced by at least one DNA primer sequence derived from SEQ ID NO:11, SEQ ID NO:12, or SEQ ID NO:9 that is diagnostic for event MON 95379 is an aspect of the invention.

DNA detection kits that contain at least one DNA primer of sufficient length of contiguous nucleotides derived from SEQ ID NO:11. SEQ ID NO:12, or SEQ ID NO:9 that, when used in a DNA amplification method, produces a diagnostic amplicon for event MON 95379 or its progeny is an aspect of the invention. A corn plant or seed, wherein its genome will produce an amplicon diagnostic for event MON 95379, when tested in a DNA amplification method is an aspect of the invention. The assay for the event MON 95379 amplicon can be performed by using an Applied Biosystems GeneAmp™ PCR System 9700, Stratagene Robocycler®, Eppendorf® Mastercycler® Gradient thermocycler or any other amplification system that can be used to produce an amplicon diagnostic of event MON 95379 as shown in Table 29.

All publications and published patent documents cited in this specification, and which are material to the invention, are incorporated herein by reference to the same extent as if each individual publication or patent application was specifically and individually indicated to be incorporated by reference.

Having illustrated and described the principles of the present invention, it should be apparent to persons skilled in the art that the invention can be modified in arrangement and detail without departing from such principles. We claim all modifications that are within the spirit and scope of the appended claims.

1. A recombinant DNA molecule comprising a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10 and a complete complement thereof.

2. The recombinant DNA molecule of claim 1, wherein the recombinant DNA molecule is derived from corn event MON 95379, a representative sample of seed comprising said event having been deposited as ATCC Accession No. PTA-125027.

3-7. (canceled)

8. A corn plant, corn plant part, corn cell, or part thereof comprising a recombinant polynucleotide molecule comprising the recombinant DNA molecule of claim 1.

9-11. (canceled)

12. A method for protecting a corn plant from insect infestation, wherein said method comprises providing in the diet of a Lepidopteran insect pest an insecticidally effective amount of cells or tissue of the corn plant comprising the recombinant DNA molecule of claim 1.

13. (canceled)

14. A method of producing an insect resistant corn plant comprising:
a) sexually crossing two different corn plants, wherein at least one of the two different corn plants comprises the recombinant DNA molecule of claim 1;
b) sampling seed or tissue from progeny of said crossing;
c) detecting the presence of a DNA segment diagnostic for corn event MON 95379 DNA in said sample from step b) to identify progeny comprising corn event MON 95379 DNA; and
d) selecting said progeny comprising corn event MON 95379 DNA.

15. A corn seed comprising a detectable amount of the recombinant DNA molecule of claim 1.

16. A nonliving corn plant material comprising a detectable amount of the recombinant DNA molecule of claim 1.

17. A microorganism comprising a detectable amount of the recombinant DNA molecule of claim 1.

18. (canceled)

19. A commodity product comprising the recombinant DNA molecule of claim 1.

20. (canceled)

21. A corn plant, corn plant part, or corn seed thereof comprising DNA functional as a template when tested in DNA amplification method producing an amplicon diagnostic for the presence of event MON 95379 DNA.

22. A method of determining the zygosity of a corn plant or corn seed comprising event MON 95379 comprising:
a) contacting a sample comprising corn DNA with a primer pair that is capable of producing an amplicon of one of the toxin coding sequences encoding Cry1B.868 or Cry1Da_7;
b) contacting said sample comprising corn DNA with a primer pair that is capable of producing an amplicon of an internal standard known to be single-copy and homozygous in the corn plant;
c) contacting the DNA sample with a probe set which contains at least a first probe that specifically hybridizes to one of the toxin coding sequences encoding Cry1B.868 or Cry1Da_7, and a second probe that specifically hybridizes to the internal standard genomic DNA known to be single-copy and homozygous in the corn plant;
d) performing a DNA amplification reaction using real-time PCR and determining the cycle thresholds (Ct values) of the amplicon corresponding to the toxin coding sequence and the single-copy, homozygous internal standard;
e) calculating the difference (ΔCt) between the Ct value of the single-copy, homozygous internal standard amplicon and the Ct value of the toxin coding sequence amplicon; and
f) determining zygosity, wherein a ΔCt of about zero (0) indicates homozygosity of the inserted T-DNA of event MON 95739 and a ΔCt of about one (1) indicates heterozygosity of the inserted T-DNA of event MON 95379.

23-25. (canceled)

26. A method of determining the zygosity of a corn plant or corn seed comprising event MON 95379 comprising:
a) contacting a sample comprising corn DNA with a set of primer pairs comprising at least two different primer pairs capable of producing a first amplicon diagnostic for event MON 95379 and a second amplicon diagnostic for native corn genomic DNA not comprising event MON 95379;
b) performing a nucleic acid amplification reaction with the sample and the set of primer pairs; and
c) detecting in the nucleic acid amplification reaction the first amplicon diagnostic for event MON 95379, or the second amplicon diagnostic for native corn genomic DNA not comprising event MON 95379, wherein the presence of only the first amplicon is diagnostic of a corn plant or corn seed homozygous for event MON 95379, and the presence of both the first amplicon and the second amplicon is diagnostic of a corn plant or corn seed heterozygous for event MON 95379; or
i) contacting a sample comprising corn DNA with a probe set which contains at least a first probe that specifically hybridizes to event MON 95379 DNA and at least a second probe that specifically hybridizes to corn genomic DNA that was disrupted by insertion of the heterologous DNA of event MON 95379 and does not hybridize to event MON 95379 DNA; and
ii) hybridizing the probe set with the sample under stringent hybridization conditions, wherein detecting hybridization of only the first probe under the hybridization conditions is diagnostic for a corn plant or corn seed homozygous for event MON 95379, and wherein detecting hybridization of both the first probe and the second probe under the hybridization conditions is diagnostic for a corn plant or corn seed heterozygous for event MON 95379.

27-28. (canceled)

29. A corn plant, corn plant part, corn seed, or corn cell comprising a recombinant polynucleotide molecule comprising nucleotides 863 to 14,027 of SEQ ID NO:10 and at least one fragment of at least 25 consecutive nucleotides of the genomic flanking sequence of SEQ ID NO:10.

30. The corn plant, corn plant part, corn seed, or corn cell of claim 29, wherein the corn plant, corn plant part, corn seed, or corn cell is insecticidal when provided in the diet of a Lepidopteran insect pest.

31. The corn plant, corn plant part, corn seed, or corn cell of claim 30, wherein the Lepidopteran insect pest is selected from the group consisting of Fall Armyworm (Spodoptera frugiperda), Corn Earworm (Helicoverpa zea), Southwestern Corn Borer (Diatraea grandiosella), Sugarcane Borer (Diatraea saccharalis), and Lesser Cornstalk Borer (Elasmopalpus lignosellus).

32. The corn plant, corn plant part, corn seed, or corn cell of claim 29, wherein the corn plant is further defined as progeny of any generation of a corn plant comprising the corn event MON 95379.

33. A method for protecting a corn plant from insect infestation, wherein said method comprises providing in the diet of a Lepidopteran insect pest an insecticidally effective amount of cells or tissue of the corn plant of claim 29.

34. The method of claim 33, wherein said Lepidopteran insect pest is selected from the group consisting of Fall Armyworm (Spodoptera frugiperda), Corn Earworm (Helicoverpa zea), Southwestern Corn Borer (Diatraea grandiosella), Sugarcane Borer (Diatraea saccharalis), and Lesser Cornstalk Borer (Elasmopalpus lignosellus).

35. A method of producing an insect resistant corn plant comprising:
a) sexually crossing two different corn plants, wherein at least one of the two different corn plants comprises a recombinant polynucleotide molecule comprising nucleotides 863 to 14,027 of SEQ ID NO:10 and at least one fragment of at least 25 consecutive nucleotides of the genomic flanking sequence of SEQ ID NO:10;
b) sampling seed or tissue from progeny of said crossing;
c) detecting the presence of a DNA segment diagnostic for the recombinant polynucleotide molecule in said sample from step b) to identify progeny comprising the recombinant polynucleotide molecule; and
d) selecting said progeny comprising the recombinant polynucleotide molecule.

36. A nonliving corn plant material or commodity product comprising a detectable amount of a recombinant polynucleotide molecule comprising nucleotides 863 to 14,027 of SEQ ID NO:10 and at least one fragment of at least 25 consecutive nucleotides of the genomic flanking sequence of SEQ ID NO:10.

37. A plant cell comprising a detectable amount of a recombinant polynucleotide molecule comprising nucleotides 863 to 14,027 of SEQ ID NO:10 and at least one fragment of at least 25 consecutive nucleotides of the genomic flanking sequence of SEQ ID NO:10.

38. The non-living corn plant material or commodity product of claim 36, wherein the commodity product is selected from the group consisting of whole or processed corn seed, animal feed comprising corn, corn oil, corn meal, corn flour, corn flakes, corn bran, corn biomass, and fuel products produced using corn and corn parts.

39. A corn plant, corn plant part, corn seed, or corn cell comprising a recombinant polynucleotide molecule comprising nucleotides 863 to 14,027 of SEQ ID NO:10 and at least one fragment of at least 25 consecutive nucleotides of the genomic flanking sequence of SEQ ID NO:10 functional as a template to produce an amplicon diagnostic for the presence of the recombinant polynucleotide molecule when tested in DNA amplification method.


SEQUENCE LISTING (28 sequences)

SEQ ID NO: 1 - DNA, 50 bp, synthetic construct
  source 1..50: mol_type = other DNA; note = 50 nucleotide sequence representing the 5' junction region of corn genomic DNA and the integrated transgenic expression cassette.
aagcaaatgtgtggtagtggtccaattttttttcaattcaaaaatgtaga

SEQ ID NO: 2 - DNA, 50 bp, synthetic construct
  source 1..50: mol_type = other DNA; note = 50 nucleotide sequence representing the 3' junction region of the integrated transgenic expression cassette and the corn genomic DNA.
gctattatgggtattatgggtaggcacatgggaatatagtgggaggcaga

SEQ ID NO: 3 - DNA, 100 bp, synthetic construct
  source 1..100: mol_type = other DNA; note = 100 nucleotide sequence representing the 5' junction region of corn genomic DNA and the integrated transgenic expression cassette.
cgtacgttcgaggtacctgtcctgcaagcaaatgtgtggtagtggtccaattttttttcaattcaaaaatgtagatgtccgcagcgttattataaaatga

SEQ ID NO: 4 - DNA, 100 bp, synthetic construct
  source 1..100: mol_type = other DNA; note = 100 nucleotide sequence representing the 3' junction region of the integrated transgenic expression cassette and the corn genomic DNA.
cgccaagaagaacgattggcaaacagctattatgggtattatgggtaggcacatgggaatatagtgggaggcagaggcgtgcctgtgcctgctcttaatt

SEQ ID NO: 5 - DNA, 200 bp, synthetic construct
  source 1..200: mol_type = other DNA; note = 200 nucleotide sequence representing the 5' junction region of corn genomic DNA and the integrated transgenic expression cassette.
tcgctagctatagcttggtagtagtcaccaacaacgacctagggagctagcgtacgttcgaggtacctgtcctgcaagcaaatgtgtggtagtggtccaattttttttcaattcaaaaatgtagatgtccgcagcgttattataaaatgaaagtacattttgataaaacgacaaattacgatccgtcgtatttataggcg

SEQ ID NO: 6 - DNA, 200 bp, synthetic construct
  source 1..200: mol_type = other DNA; note = 200 nucleotide sequence representing the 3' junction region of the integrated transgenic expression cassette and the corn genomic DNA.
aggcgcgggtaccgtcggtccgggcctagtaggccaagcaggacgtggcgcgccaagaagaacgattggcaaacagctattatgggtattatgggtaggcacatgggaatatagtgggaggcagaggcgtgcctgtgcctgctcttaatttgagctcctcccctggccctgatagggcatgtgcatgcgcacaataatca

SEQ ID NO: 7 - DNA, 1160 bp, synthetic construct
  source 1..1160: mol_type = other DNA; note = 1160 nucleotide sequence representing the 5' junction region of corn genomic DNA and the integrated transgenic expression cassette.
cgcagcgcaggtccgtgtggcctgtttgttttggcttctggtagcttctggccaccaaaagctgctgcggactgccaaacgctcagcttttcagccagcttctataaaatttgttggggggcaaaaaccatccaaaatcaacataaacacataatcagttgagtcgttgtaatagtaggaattcgtcactttctagatcctgagccctatgaacaattttatcttcctccacacgtaatcgtaatgatattcagattctccccacagccagattctcctcacagccagattttcagaaaagctggtcagaaaaaagctgaaccaaacaggccctaggacacacataggaatggatcaaaaaacatagcgccgtacccgtacgtacgtgcagtcacagtcagctgctagctagctagctgcgccggcggcggcgttgtcggtcttgactcaacgacgtccgcggctggcgccgtcggccagcggcttgttgccgccggtcccatccggcggagtccggctgccagtggccggcggcggcttcttcttcgtcgtcgtcctgccctttgccatccccgctgccacctggacgagggttctggctcccgtcgacgtcgacgagagacgtggcgccgcatttggctgccgccgtatcgtcgtcgtcgtcgagattgaaatcgagagggagcgcgcgggatggctgccgccggtggcgcgacatggtctcgcccggccggcgcctgatcgacttctcttccgccgccttcgctagctatagcttggtagtagtcaccaacaacgacctagggagctagcgtacgttcgaggtacctgtcctgcaagcaaatgtgtggtagtggtccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcgatgcggccaccacgagtcgacataacttcgtatagcatacattatacgaagttatagcggccgcaaatcaacctcactctatttaaatgaggtggtaggatt

SEQ ID NO: 8 - DNA, 1178 bp, synthetic construct
  source 1..1178: mol_type = other DNA; note = 1178 nucleotide sequence representing the 3' junction region of the integrated transgenic expression cassette and the corn genomic DNA.
taagggtcccaagtaaggccggccaagtaacggtccgaagtaaggcgcgggtaccgtcggtccgggcctagtaggccaagcaggacgtggcgcgccaagaagaacgattggcaaacagctattatgggtattatgggtaggcacatgggaatatagtgggaggcagaggcgtgcctgtgcctgctcttaatttgagctcctcccctggccctgatagggcatgtgcatgcgcacaataatcattggagctgcctgaatgatttgcatgcgctcgtgtaaaacgttcacggtccatgccagtccaagaccgtcggcaagagatgagggccatgcgcagtacgtacatttgtatacatatacatatttttttggatgatgcgatgcgtgtgcatatctggctgcgccgctccctcgtccatcgtcgtcgcggtgtacaacgaattttccgtgggatgggctgaacttataatgggcctactgggcgggccgtatgaaatagcacacaattcaccctgcttttctcaccaggaatgcattttcgcgggagccatcgtcctaacttggcagacagacacacaaacacaggacaggagttcgccaaccccagcatgagagaaacctatccggacagctcctccagcctatgggccgttgggccttcagcctcagcgccccggccccgtgggcgggcaaaacactccacagtccacacggctgctgctgctgcccttgttccgtcggacctggggtaactttatagctatgatgttcgcccttcccaaccaatgtgcgagcgttaaggatgttaagtaacactagttaggattatgtggccaaatacctactttgctaggaacttgtacacccctcataatatagagaaaagaaagatgccagtggtccaactctataccatgtgtctcgtgtgtttgttatgtcgtgcttatgtgaagggagacgagtcttctacagcttctcacgcctaatctgttgacgggagagaagagagcggatctggtgatccgtggtaacgtagttctcaacaaatgacacattgagcacaccgttgtcatctcctcagctaaacattgagggggatttttttttaagaaaggctgagattcccttacgctccctttggcagggctcgtccaacagcttcttgaaaaattctataa

SEQ ID NO: 9 - DNA, 13318 bp, synthetic construct
  source 1..13318: mol_type = other DNA; note = Sequence corresponding to the transgenic inserted T-DNA of corn event MON 95379.
ttttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcgatgcggccaccacgagtcgacataacttcgtatagcatacattatacgaagttatagcggccgcaaatcaacctcactctatttaaatgaggtggtaggatttgccgcgtgagacctattaccctgttatccctagtttaaacacctgcaggaaaagattaattaacatcgatcatcatgttcaattgacagcaaattaaactagttatatatagggttcatgagatgagagtagctaattattaacacaagtggaagaaacttttatttgacggtatacaaataacaagatacagagatcaggctgatggatcaatcatatcatatcagtgatagatcgatcgttcgacacatatatatgtatgacaaaacatagtataagtacgtgcgaaagggtttatatatatatggatattgatgcatggtgtaatggtatggtaattaagggatcggaggatcgattaatgggtccctatcactcctccatcagtagcagctcgaccgagtccacgatgaaagtgccctccgtctcgccgatctcgatccagactttgtcggtctccgggaagtactccagctccttggtgacgtacccggcgggcagcggagtgtagtcaccgtagccacggttggactcgcaagggttgtccctccgtccgtcggtgtacgccttctcctcgtaagcggacgcataatccgcaggcacgctgctgttggactcgtacgcgccatcatagccgcgattccgggaagtgtaagtgccctcgtactcctcttgcgtggcagtgtagtcgttgcaagtaacggtgttgttcgggtacacctcctcctccacacagttcgagaacttgagctcgtcggtgttgttctcgatctcgtggatagtcacgcagccttcaccgtagccttccttgtacgcagtcacacggaggatgtagccgcgaccagggcagacgcggacctcctggctcacctctgcctcccactccggcacgaccagcacggaccggtggttgttctgctcctcgacgtccacatggcccttgacgttccagcaactcagcccgttgttgaagtcaccgttcttaataacattcctggcatcgtacaggctgaatgctgtgaagatgcggccctccagttcctcgaagatcgccgcgttcacaccagggatcactgacagctcgggcaggtacgcctcacggatgctgtggacccgcttgtcagccgcgtggatcatggcgatgttggtgtcagcctggaggcggtcgtactggctgttgacgaagagtgcgtccactgactccttggcctccttgtacacgatgttggtctcccactccagcttctcccgcttgtctcgccatttcttctcggcgcgcttcacacgagcgagggcctcgcccacgagaggcttctcctcaaggaactccaggttgccgaggcgggcatgtccgtcctgggtcttaatcttgaagataacccagacacccaggtcctcgttgaggtcggtgcagcccacgtcgatgtccagagagaagtggtgcgagtggtgagcgcactttccgattggagagggagcgctcaatggccaaagtgagcccgtgcctggcacgttcactgtctcgtgcttggcgttgtatcggatcaggtagatttccaggtcctgactatcctcgatgtacccgcgaagctggtagcgggtgtacgccttcaatttactctcgtctatcttctggtagagatacgtcgggtagcattcgtcgaaagtcccgagcagcgtcacatagttctccttgaacacatcgtcgcctccctggatggtgatgtctgtactcccgcgccaaccacggtcgagctggcggttgatgccacggaagttcgggtcctggagcaaattccgctcgtcactcagacgcttcgcgtgtttgaccttctcggacagctccttcttctcgtcgaggcagaactcgtcggacaggcactccacaaggttggacacttggtcaatgtgatagtccgtgacgtcggtcttgagcccaatctggttgctgctggtgaacagttcgttgacagccttctgagcccgctccaggtcagactcggcctcgaacgtcgcatccgccaggatgatctcaatcttgtcgatgtacagttcgccagagctgatggagccagcaccgaacagcggttgctcggagatcccaatgatgtcagggttcgctctgaatgagaaagggttcgagaaatcagtgtaccggaaggttcggctagtcaagttctcgcctatctccatcgtcttctggagaggcatgttgactgagacttggcctcccacgcctgtggacgcggctccggtcaggacaatgaccctcgcgtcgcggcttgaagcgtagcggaacctcagacggtaacgctgggtgatcggcgagttgatgttcacttgcaacgaaacgaagtcgccgaaagtgttgcgccgcagtatgtctccaccggtgaagcccggaccggtgatgacgctcgtgccaccccacacacggaagcccttcacgagcgggatctggttaatggagtcggaagagatcgtgttggtccggtcggcgctgcggtgtgtccaggagtacaccggcgcacgcagcgtgttgccagagatgagccggatgttggacaggcggtggctgtaggactcgtagttcgggcgttccgtggtctcgggcggcagctcagtttcactgtcgaagagctgggtgccgacgcccgtgtacccgatggtgtagagcaatgacccgcgcagggagttcaacgggttacgccagttgaaccgggcccacgggacgccgttgacgggcgttgtcagaaggatgttaatgcccgcgaacgactcagtccggtaaacgtcgcggctcgtgaactgtagcgtaactgggttgatggaggtgttggtgttgccgtgggtactggtgctcaagctgccacggatcgtcctactctccaatcggtgcccgacccagtagttcatgtactgtgtgttggaccagcgtgagagcacggagaagatcgtgagctgctcgggaaagtcgagcaggtgaggagggcggatgacggcggcctcgattgccgagaaggagggtgcgttgttgttgaaccaattcgtggatgcgaaaccggagggtgcgttcgtccggccgattgggtctgtgtagatctcccgcgtgagttgcgcgctagtgttcattgggtacacccgtgtgtcgtaagacgggaatagcgccaccaggtctaggacacccaaagtcaagtcgcggcggaactggttgtaacggagccatgactcagcgttggtgccgcgcaggttgttgagccctgtgttgtaccatcgagcgcagtagtcactgtactcacgggtcttctccacttggcgctcgtagtaacgctggatttcttgagatgtgagtccgaactcggagccgaacaggctggcgtcccgcagcaggagcagatgtaagttggcggcctgggcgtacaccataaggagtggaacttcctggttcctgatggcgaacagcggcatagcgttcaggaagtcgagttcgagagcgatgtactgggtgtagagtaccgagcgtgttcgcgcgtcatccctgttctccaaccagtcctccagtgactgctggtatgcccgaaaggagtttcccaggccctgtagcctggccagagcagtatcgcgggtgttctccgtgacttgctgccggatcaactgctccacatgctccaggaagatctcccacgggtcacgaccgcgaggccacagctcgcccacaaggaacgagtagaaggacgcgatctgaccggcgaatgggacaccgaggacgccgaggatacggcccgcgatgttgatgccggtctgaaccgtgctggccgacacgaatgggtcgatgttgttgccctcggctatgcagagggagtcctcgatgcgcgcgtcggtggacaggttcatctgcgcggagtggtttgaaacagcagggatcgagagggcgttgatgatctcgttctcgttcttccggttgctcgtcatggtggtggccggccaagtaacggtccgacctgcagaagtatcaccaaacaacagggtgagcatcggacaaaagaaacagtaccaagcaaataaatagcgtatgaaggcagggctaaaaaatccacatatagctgctgcatatgccatcatccaagtatatcaagatcaaaataattataaaacatacttgtttattataatagataggtactcaaggttagagcatatgaatagatgccgcatatgccatcatgtatatgcatcagtaaaacccacatcaacatgtatacctatcctagatcaatatttccatccatcatcttaaactcgtaactatgaagatgtatggcacacacataaagatacaaaattaataaatccaccaggtagtttgaaacagtattctactccgatctagaacgaccgcccaaccagaccacatcatcacaaccaagcgaaaaaaagcatctctgtatatgcatcagtaaaacccgcatcaacatgtatacctatcctagatcgatatttccatccatcatcttaaactcgtaactatgaagatgtaggcacatacatacagatcctttaataaatccaccaggtagtttgaaacagtattctactccgatctagaacgaccctttatcctttaataaatccaccaggtagtttgaaacagtattctactccgatctagaacgaccgcccaaccagaccatatcatcacaaccaagcgagaaaaaaagcatctctgtatatgcatcagtaaaacccgcatcaacatgtatacctatcctagatcgatatttccatccatcatcttaaactcgtaactatgaagatgtatggcacacacatacagatacaaaattaataaatccaccaggtagcttgaaacagtatcctactccgatctagaacgaccgcccaaccagaccacatcatcacaaacatgaacatgaacatgtttgctctaacacaaacatgaacagaagtagaactaccgggccctaaccatggaccggatcgccgatctagagaaggtagagagagggggggggggaggatgagcggcgtaccttgaagcggaggtgccgacggctggatttgggggagatctggttgcgtgtgtgtgcgctccgaacgaacacgaggttggggaaagagggtgtggagggggtgtctatttattacggcgggcgaggaagggaaagcgaaggagcggtgggaaaggaatcccccgtagctgccggtgccgtgagaggaggaagaggccgcctgccgtgccgcctcacgtctgccgctccgccacgcaatttctggatgccgacagcggagcaagtccaacggtggagcggaactctcgagaggggtccagaggcagctacagagatgccgtgccgtctgcttcgcttggcccgacgcgacgctgctggttcgctggttggtgtccgttagactcgtcgacggcgttgaacagcctgtcattatctactcgaaacaagaaaaatgtttgcttagtttttttatttcttaaagggtatttgttttatttgtagtcaatttattttatttcattttatatctaaattattaaataaaaaactaaaatagagttttagttttaaaaaatttagagactaaaaagaataaaatggatgtactaaaaattagtctatagaaaccatcaaccctaaatcctaaatggatgtactaataaaatggatgaagtattatataggtcaagctatttgcaaaaaaaacagagagatcacatgcacactaaaaagataaaactgtagattgtcaaaatattcaattatcctttagaccatgtctagcagtttatttatatgatcctctaaaacactaatattattttagtattatagactatattatttgtagagtgaagtttaaatatatgtatagagatagataaactacacttcaaataagtgtgacaaaaaaatatgtggtaattttttatactttagacatgcaatgctctttatctctagagaggggcacgacgcatgcaagcttgttaacgcggccgctaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattagcttgagctcaggatttagcagcattccagattgggttcaatcaacaaggtacgagccatatcactttattcaaattggtatcgccaaaaccaagaaggaactcccatcctcaaaggtttgtaaggaaggtcgaggccgctgtactgtcatattgtcgtggtttttcaattgctgtacctgatgcaaacgtaatgggtttactaatcttgcacccgccggcttcaaaatgaagagtgctaatttggtccacgtcaccatcaccggttcgaactgtctagaatggcaggcaaagatgattggacaggcatgcagggaaaaagagcaccgttgacgatgtatgcgagttcccaccattgcgagcaatgattatcagccacacgacttactcttcagagctaaccactgccatgcagagaaaaagtgaatcatattgtcatgatctacaacgaagtgaaacaatcaggcatgctaaagtgctgaaactttactgatctctcatgttggacaacaaagaatacgggaatacatcagcaacgcaactcttgagctttgcttgccgaatgaccagctagaatttccaagcatttacagaaacatgactttaagtttcagaaaaacaaatacaaggccactaaataagcgtggggataacatatcctccagatgacaggcaatctgcaacttgcagccattcaaatgtacgattaacaaaatatttaagcgccacatgagataatatatcctccaattagggcctttagtattgtcattagctcataaccatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccgcgagagggcgacgccatgtgtataaaaggggcctaagctgagtggacttgctgcatcagattagtaagcaatctcaagcgcagagagccaaagctttcggtgtagctcgaagagcaaagcgaaggcaaggcgcgcctgtaacctacctaccaacctcgccattcctctccaaactgttgtgctgctgtctagatctcccacactacactagttactcctcgtagatctcggctacctggctcaagatccggggtcagatccgggtccggggattttctttgtgccctatggctgtattttggcgtctgtggctgatgacagcgtgtgttctcgagtgcggatgcaatctgagttatataggcaaatggccttgtcaactcgggcagcggcattgctttgctcagtgtgtttgaatgtgctgaaattcatgtagtaggctgtaggctgtgcatttcttgatttgcgtcttgcataattcactggtggattttctaaacctaacaagtttaaaattagaccattcaaccaaagacaggaggaataagtgaagctgttgtagtcacagcttatggccgatccaaaatttgttaggaatgtgaatatgtgatgctacaaacatatccttgtaagctaccatgctatttatcatgttccatcatggtgattggtgagcactcatgaaaatttcagatccaaacctagtgttacatgtggatttgtgctctgcaatctatcgccagtaataaaatggttgagtgatccagctactacaaaatcacattgcatactttttttttttgtagattatgcatcctggttttgggtggtgggttcctgatgtcaggaatataaatttagcctgctgatttaggtagcactgccggtgcacactttggtttttgaatacttgtagtcttccagcttcttgtagaactggtacaatgtgggccatatataagaagggctgtcaactagcacatgctcactaattagtctaaacatttatgtttttattcattcaggtcaggtgcaatcatagaagtagttaatgacaatactttagttgttctaatattatttatgtatggactcaaattaacatgcaaaacatatgagattagtggcatgcattctttttcttaatagtggaaaatacgagataatgataactgtgaagctctgttagtactcttcattactctatttgagtggcagcatatctcatgctagccataaagcaagttctagacgtattctgttgttaattacttgtagctatataacccaacctagtcattccagcttatgtctcttagagatcatgtttattagcacctcaagatttcctctgcacagtatagtaactatcgaaaaagatattatttctttgtttttaattgacaaccttcacgtgctacttatttttgcagcttggccggccaccaaccatggctgagatcaacaaccagaaccagtgcgtcccgtacaactgcctgagcaaccctaaggagatcatcctgggtgaggaacgcctggagaccggcaacaccgtagccgacattagcctgggcctcatcaacttcctctacagcaacttcgtgcccggcggtggcttcatcgtgggcctcctggagcttatctggggcttcatcggcccgtcccagtgggacatcttcctcgcccagatcgagcaactgatcagccagcggatcgaggagttcgctaggaaccaggccatctcccgcctggagggactctccaacctctacaaggtgtacgtgcgcgcgttcagcgactgggagaaggacccgaccaacccggccctccgcgaggaaatgcgtatccagttcaacgatatgaactcggccctcatcaccgccatcccgctcttccgcgtgcagaactacgaggtggccctcctgtccgtgtacgttcaagccgccaacctccacctctccatcctccgcgacgtgagcgtgttcggcgagcgctggggctacgacaccgccaccatcaacaaccgctactccgacctcacctccctcatccacgtttacaccaaccactgcgtggacacgtacaaccagggcctccgccgcctggagggccgcttcctctccgactggatcgtgtacaaccgcttccgccgccagctcaccatctccgtcctggacatcgtcgccttctttcccaactacgacatccgcacctaccctatccagaccgccacccagctcacccgcgaggtctacctcgacctcccgttcatcaacgagaacctcagcccggccgccgtctacccgaccttctccgccgctgagtccgccatcattcgcagcccgcacctcgtggacttcctcaactccttcaccatctacaccgactccctcgcccgcagcgcctactggggcggtcacctcgtgaactccttccgcaccggcaccactaccaacctcatccgcagcccgctctacggccgcgagggcaacaccgagcgcccggtgaccatcaccgccagcccgagcgtgcccatcttccgcaccctcagctaccccaccggcctggacaacagcaaccctgtggcgggcatcgagggcgtggagttccagaacaccatctccaggagcatctaccgcaagagcggccctatcgacagcttcagcgagctgcctcctcaggacgccagcgtgagccctgccatcggctacagccacaggctgtgccacgccaccttcctggagcgcatcagcggccctcgcatcgcgggcaccgtgttctcgtggacccaccgcagcgcctctcctacgaacgaggtgtctcctagtcgcatcacccagatcccttgggtcaaggcccacaccctggctagtggcgctagtgtcatcaagggccctggcttcaccggtggtgacatcctgaccaggaactctatgggcgagctgggcactctgagggtcactttcactggccgcctgcctcagtcttactacatccgcttccgctacgctagtgtcgctaaccgctctggtactttccgctactctcagcctccgtcttacggtatctctttccctaagactatggacgctggtgagcctctgaccagtaggagcttcgctcacactactctgttcactcctatcactttctctagggctcaggaggagttcgacctgtacatccagtctggtgtgtacatcgacaggatcgagttcatccccgtgaccgccacgttcgaggccgagtacgaccttgagcgcgcccagaaggtggtgaacgccctcttcactagcactaaccagctaggcctgaagactgacgtgaccgactaccacatcgaccaagtgagcaacctagtggcctgcctctccgacgagttctgcctcgacgagaagcgcgagctgtccgagaaggtgaagcacgccaagcgcctctccgacgagcgcaacctgctccaggaccccaacttcaggggcatcaacaggcagcccgaccgcggctggcgcggctccaccgacatcaccatccagggcggtgacgacgtattcaaggagaactacgttaccctccccggcaccttcgacgagtgttaccccacctacctctaccagaagatcgacgagtccaagctgaaggcctacacccgctaccagctccgcggctacatcgaggactcccaggacctggaaatctacctcatccgctacaacgccaagcacgagatcgtgaacgtgcctggcaccggcagcctctggcctctcagcgtggagaaccagatcggcccttgcggcgagcctaaccgctgcgcccctcacctcgagtggaaccctgacctccactgctcgtgcagggacggcgagaagtgcgcccaccatagccaccacttctctctggacatcgacgtgggctgcaccgacctgaacgaggacctgggcgtgtgggttatcttcaagatcaagacccaggacggtcacgccaggctgggtaacctggagttccttgaggaaaagcctctgctgggtgaggccctggccagggtcaagagggctgagaagaaatggagggataagagggagaccctgcagctggagaccactatcgtctacaaggaggctaaggagtctgtcgatgctctgttcgtcaactctcagtacgatagactgcaagctgataccaacatcgctatgatccacgctgcggataagcgggtccaccggatccgggaggcttaccttccggagctttctgtcatcccgggtgtcaacgctgcgatcttcgaggaacttgaggaacggatcttcactgcgtttagtctttacgatgcgcggaacatcatcaagaacggggacttcaacaatggtctgctgtgctggaacgtcaagggtcatgtcgaggtcgaggaacaaaacaatcatcgtagtgtccttgtcattcctgagtgggaggcggaggtctctcaagaggtccgtgtttgcccggggcgtgggtacattcttcgtgttactgcgtacaaggaggggtacggggaggggtgcgttactattcatgagattgagaacaatactgatgagcttaagttcaacaattgtgttgaggaggaggtttacccgaacaatactgttacgtgcatcaactacacggcaacgcaagaggaatacgaggggacgtacacctcgcgtaatagagggtatgatgaggcgtacggaaacaacccgtcggttccagcagattatgcctcggtttatgaggagaagtcgtacacggatagacgacgcgagaatccatgtgagtcaaatcgaggatacggagattacacaccattaccagcaggatacgttacaaaggagttggaatacttcccggaaacagataaagtttggattgaaatcggagaaacagaaggaacattcatcgtcgactcagtagaattgttgttgatggaagaatgatagggaccccggacccgccaaaaccattgcaaagactatagtttggggtggagtatacttggttgtgtacatgcctgcgtgttccattgtacacacaaaacctagccacctcttgactcttgagtgtatgcttgttatccgtgtgttgaagtttgtaagaggcaccatcactatagatgatggcttgtgtccctctttcatcaagattgaataatatatgctactttgagagcgctatcctgcttgcctgattgtgttaatacttacatccgtcccacactcccacaatataaggaaataaggtattttggcagtttagagcaaaattcccttatatttttgggacggatgtcctcttttctgcatttttttatgttcatatgttcctgaagagtaaggtggatcttgatcaacctgtcggtttatggtgattgatttgagtggaatagaatgggccaacgtccggcatacagttatgcttcagttaattaaaagtaagggtcccaagtaaggccggccaagtaacggtccgaagtaaggcgcgggtaccgtcggtccgggcctagtaggccaagcaggacgtggcgcgccaagaagaacgattggcaaacagctattatgggtattatgggtaggc

SEQ ID NO: 10 - DNA, 15216 bp, synthetic construct
  source 1..15216: mol_type = other DNA; note = Sequence corresponding to the contig nucleotide sequence of the 5' genomic flanking DNA nucleotide sequence, the inserted T-DNA sequence in event MON 95379, and the 3' genomic flanking DNA nucleotide sequence.
cgcagcgcaggtccgtgtggcctgtttgttttggcttctggtagcttctggccaccaaaagctgctgcggactgccaaacgctcagcttttcagccagcttctataaaatttgttggggggcaaaaaccatccaaaatcaacataaacacataatcagttgagtcgttgtaatagtaggaattcgtcactttctagatcctgagccctatgaacaattttatcttcctccacacgtaatcgtaatgatattcagattctccccacagccagattctcctcacagccagattttcagaaaagctggtcagaaaaaagctgaaccaaacaggccctaggacacacataggaatggatcaaaaaacatagcgccgtacccgtacgtacgtgcagtcacagtcagctgctagctagctagctgcgccggcggcggcgttgtcggtcttgactcaacgacgtccgcggctggcgccgtcggccagcggcttgttgccgccggtcccatccggcggagtccggctgccagtggccggcggcggcttcttcttcgtcgtcgtcctgccctttgccatccccgctgccacctggacgagggttctggctcccgtcgacgtcgacgagagacgtggcgccgcatttggctgccgccgtatcgtcgtcgtcgtcgagattgaaatcgagagggagcgcgcgggatggctgccgccggtggcgcgacatggtctcgcccggccggcgcctgatcgacttctcttccgccgccttcgctagctatagcttggtagtagtcaccaacaacgacctagggagctagcgtacgttcgaggtacctgtcctgcaagcaaatgtgtggtagtggtccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcgatgcggccaccacgagtcgacataacttcgtatagcatacattatacgaagttatagcggccgcaaatcaacctcactctatttaaatgaggtggtaggatttgccgcgtgagacctattaccctgttatccctagtttaaacacctgcaggaaaagattaattaacatcgatcatcatgttcaattgacagcaaattaaactagttatatatagggttcatgagatgagagtagctaattattaacacaagtggaagaaacttttatttgacggtatacaaataacaagatacagagatcaggctgatggatcaatcatatcatatcagtgatagatcgatcgttcgacacatatatatgtatgacaaaacatagtataagtacgtgcgaaagggtttatatatatatggatattgatgcatggtgtaatggtatggtaattaagggatcggaggatcgattaatgggtccctatcactcctccatcagtagcagctcgaccgagtccacgatgaaagtgccctccgtctcgccgatctcgatccagactttgtcggtctccgggaagtactccagctccttggtgacgtacccggcgggcagcggagtgtagtcaccgtagccacggttggactcgcaagggttgtccctccgtccgtcggtgtacgccttctcctcgtaagcggacgcataatccgcaggcacgctgctgttggactcgtacgcgccatcatagccgcgattccgggaagtgtaagtgccctcgtactcctcttgcgtggcagtgtagtcgttgcaagtaacggtgttgttcgggtacacctcctcctccacacagttcgagaacttgagctcgtcggtgttgttctcgatctcgtggatagtcacgcagccttcaccgtagccttccttgtacgcagtcacacggaggatgtagccgcgaccagggcagacgcggacctcctggctcacctctgcctcccactccggcacgaccagcacggaccggtggttgttctgctcctcgacgtccacatggcccttgacgttccagcaactcagcccgttgttgaagtcaccgttcttaataacattcctggcatcgtacaggctgaatgctgtgaagatgcggccctccagttcctcgaagatcgccgcgttcacaccagggatcactgacagctcgggcaggtacgcctcacggatgctgtggacccgcttgtcagccgcgtggatcatggcgatgttggtgtcagcctggaggcggtcgtactggctgttgacgaagagtgcgtccactgactccttggcctccttgtacacgatgttggtctcccactccagcttctcccgcttgtctcgccatttcttctcggcgcgcttcacacgagcgagggcctcgcccacgagaggcttctcctcaaggaactccaggttgccgaggcgggcatgtccgtcctgggtcttaatcttgaagataacccagacacccaggtcctcgttgaggtcggtgcagcccacgtcgatgtccagagagaagtggtgcgagtggtgagcgcactttccgattggagagggagcgctcaatggccaaagtgagcccgtgcctggcacgttcactgtctcgtgcttggcgttgtatcggatcaggtagatttccaggtcctgactatcctcgatgtacccgcgaagctggtagcgggtgtacgccttcaatttactctcgtctatcttctggtagagatacgtcgggtagcattcgtcgaaagtcccgagcagcgtcacatagttctccttgaacacatcgtcgcctccctggatggtgatgtctgtactcccgcgccaaccacggtcgagctggcggttgatgccacggaagttcgggtcctggagcaaattccgctcgtcactcagacgcttcgcgtgtttgaccttctcggacagctccttcttctcgtcgaggcagaactcgtcggacaggcactccacaaggttggacacttggtcaatgtgatagtccgtgacgtcggtcttgagcccaatctggttgctgctggtgaacagttcgttgacagccttctgagcccgctccaggtcagactcggcctcgaacgtcgcatccgccaggatgatctcaatcttgtcgatgtacagttcgccagagctgatggagccagcaccgaacagcggttgctcggagatcccaatgatgtcagggttcgctctgaatgagaaagggttcgagaaatcagtgtaccggaaggttcggctagtcaagttctcgcctatctccatcgtcttctggagaggcatgttgactgagacttggcctcccacgcctgtggacgcggctccggtcaggacaatgaccctcgcgtcgcggcttgaagcgtagcggaacctcagacggtaacgctgggtgatcggcgagttgatgttcacttgcaacgaaacgaagtcgccgaaagtgttgcgccgcagtatgtctccaccggtgaagcccggaccggtgatgacgctcgtgccaccccacacacggaagcccttcacgagcgggatctggttaatggagtcggaagagatcgtgttggtccggtcggcgctgcggtgtgtccaggagtacaccggcgcacgcagcgtgttgccagagatgagccggatgttggacaggcggtggctgtaggactcgtagttcgggcgttccgtggtctcgggcggcagctcagtttcactgtcgaagagctgggtgccgacgcccgtgtacccgatggtgtagagcaatgacccgcgcagggagttcaacgggttacgccagttgaaccgggcccacgggacgccgttgacgggcgttgtcagaaggatgttaatgcccgcgaacgactcagtccggtaaacgtcgcggctcgtgaactgtagcgtaactgggttgatggaggtgttggtgttgccgtgggtactggtgctcaagctgccacggatcgtcctactctccaatcggtgcccgacccagtagttcatgtactgtgtgttggaccagcgtgagagcacggagaagatcgtgagctgctcgggaaagtcgagcaggtgaggagggcggatgacggcggcctcgattgccgagaaggagggtgcgttgttgttgaaccaattcgtggatgcgaaaccggagggtgcgttcgtccggccgattgggtctgtgtagatctcccgcgtgagttgcgcgctagtgttcattgggtacacccgtgtgtcgtaagacgggaatagcgccaccaggtctaggacacccaaagtcaagtcgcggcggaactggttgtaacggagccatgactcagcgttggtgccgcgcaggttgttgagccctgtgttgtaccatcgagcgcagtagtcactgtactcacgggtcttctccacttggcgctcgtagtaacgctggatttcttgagatgtgagtccgaactcggagccgaacaggctggcgtcccgcagcaggagcagatgtaagttggcggcctgggcgtacaccataaggagtggaacttcctggttcctgatggcgaacagcggcatagcgttcaggaagtcgagttcgagagcgatgtactgggtgtagagtaccgagcgtgttcgcgcgtcatccctgttctccaaccagtcctccagtgactgctggtatgcccgaaaggagtttcccaggccctgtagcctggccagagcagtatcgcgggtgttctccgtgacttgctgccggatcaactgctccacatgctccaggaagatctcccacgggtcacgaccgcgaggccacagctcgcccacaaggaacgagtagaaggacgcgatctgaccggcgaatgggacaccgaggacgccgaggatacggcccgcgatgttgatgccggtctgaaccgtgctggccgacacgaatgggtcgatgttgttgccctcggctatgcagagggagtcctcgatgcgcgcgtcggtggacaggttcatctgcgcggagtggtttgaaacagcagggatcgagagggcgttgatgatctcgttctcgttcttccggttgctcgtcatggtggtggccggccaagtaacggtccgacctgcagaagtatcaccaaacaacagggtgagcatcggacaaaagaaacagtaccaagcaaataaatagcgtatgaaggcagggctaaaaaatccacatatagctgctgcatatgccatcatccaagtatatcaagatcaaaataattataaaacatacttgtttattataatagataggtactcaaggttagagcatatgaatagatgccgcatatgccatcatgtatatgcatcagtaaaacccacatcaacatgtatacctatcctagatcaatatttccatccatcatcttaaactcgtaactatgaagatgtatggcacacacataaagatacaaaattaataaatccaccaggtagtttgaaacagtattctactccgatctagaacgaccgcccaaccagaccacatcatcacaaccaagcgaaaaaaagcatctctgtatatgcatcagtaaaacccgcatcaacatgtatacctatcctagatcgatatttccatccatcatcttaaactcgtaactatgaagatgtaggcacatacatacagatcctttaataaatccaccaggtagtttgaaacagtattctactccgatctagaacgaccctttatcctttaataaatccaccaggtagtttgaaacagtattctactccgatctagaacgaccgcccaaccagaccatatcatcacaaccaagcgagaaaaaaagcatctctgtatatgcatcagtaaaacccgcatcaacatgtatacctatcctagatcgatatttccatccatcatcttaaactcgtaactatgaagatgtatggcacacacatacagatacaaaattaataaatccaccaggtagcttgaaacagtatcctactccgatctagaacgaccgcccaaccagaccacatcatcacaaacatgaacatgaacatgtttgctctaacacaaacatgaacagaagtagaactaccgggccctaaccatggaccggatcgccgatctagagaaggtagagagagggggggggggaggatgagcggcgtaccttgaagcggaggtgccgacggctggatttgggggagatctggttgcgtgtgtgtgcgctccgaacgaacacgaggttggggaaagagggtgtggagggggtgtctatttattacggcgggcgaggaagggaaagcgaaggagcggtgggaaaggaatcccccgtagctgccggtgccgtgagaggaggaagaggccgcctgccgtgccgcctcacgtctgccgctccgccacgcaatttctggatgccgacagcggagcaagtccaacggtggagcggaactctcgagaggggtccagaggcagctacagagatgccgtgccgtctgcttcgcttggcccgacgcgacgctgctggttcgctggttggtgtccgttagactcgtcgacggcgttgaacagcctgtcattatctactcgaaacaagaaaaatgtttgcttagtttttttatttcttaaagggtatttgttttatttgtagtcaatttattttatttcattttatatctaaattattaaataaaaaactaaaatagagttttagttttaaaaaatttagagactaaaaagaataaaatggatgtactaaaaattagtctatagaaaccatcaaccctaaatcctaaatggatgtactaataaaatggatgaagtattatataggtcaagctatttgcaaaaaaaacagagagatcacatgcacactaaaaagataaaactgtagattgtcaaaatattcaattatcctttagaccatgtctagcagtttatttatatgatcctctaaaacactaatattattttagtattatagactatattatttgtagagtgaagtttaaatatatgtatagagatagataaactacacttcaaataagtgtgacaaaaaaatatgtggtaattttttatactttagacatgcaatgctctttatctctagagaggggcacgacgcatgcaagcttgttaacgcggccgctaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattagcttgagctcaggatttagcagcattccagattgggttcaatcaacaaggtacgagccatatcactttattcaaattggtatcgccaaaaccaagaaggaactcccatcctcaaaggtttgtaaggaaggtcgaggccgctgtactgtcatattgtcgtggtttttcaattgctgtacctgatgcaaacgtaatgggtttactaatcttgcacccgccggcttcaaaatgaagagtgctaatttggtccacgtcaccatcaccggttcgaactgtctagaatggcaggcaaagatgattggacaggcatgcagggaaaaagagcaccgttgacgatgtatgcgagttcccaccattgcgagcaatgattatcagccacacgacttactcttcagagctaaccactgccatgcagagaaaaagtgaatcatattgtcatgatctacaacgaagtgaaacaatcaggcatgctaaagtgctgaaactttactgatctctcatgttggacaacaaagaatacgggaatacatcagcaacgcaactcttgagctttgcttgccgaatgaccagctagaatttccaagcatttacagaaacatgactttaagtttcagaaaaacaaatacaaggccactaaataagcgtggggataacatatcctccagatgacaggcaatctgcaacttgcagccattcaaatgtacgattaacaaaatatttaagcgccacatgagataatatatcctccaattagggcctttagtattgtcattagctcataaccatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccgcgagagggcgacgccatgtgtataaaaggggcctaagctgagtggacttgctgcatcagattagtaagcaatctcaagcgcagagagccaaagctttcggtgtagctcgaagagcaaagcgaaggcaaggcgcgcctgtaacctacctaccaacctcgccattcctctccaaactgttgtgctgctgtctagatctcccacactacactagttactcctcgtagatctcggctacctggctcaagatccggggtcagatccgggtccggggattttctttgtgccctatggctgtattttggcgtctgtggctgatgacagcgtgtgttctcgagtgcggatgcaatctgagttatataggcaaatggccttgtcaactcgggcagcggcattgctttgctcagtgtgtttgaatgtgctgaaattcatgtagtaggctgtaggctgtgcatttcttgatttgcgtcttgcataattcactggtggattttctaaacctaacaagtttaaaattagaccattcaaccaaagacaggaggaataagtgaagctgttgtagtcacagcttatggccgatccaaaatttgttaggaatgtgaatatgtgatgctacaaacatatccttgtaagctaccatgctatttatcatgttccatcatggtgattggtgagcactcatgaaaatttcagatccaaacctagtgttacatgtggatttgtgctctgcaatctatcgccagtaataaaatggttgagtgatccagctactacaaaatcacattgcatactttttttttttgtagattatgcatcctggttttgggtggtgggttcctgatgtcaggaatataaatttagcctgctgatttaggtagcactgccggtgcacactttggtttttgaatacttgtagtcttccagcttcttgtagaactggtacaatgtgggccatatataagaagggctgtcaactagcacatgctcactaattagtctaaacatttatgtttttattcattcaggtcaggtgcaatcatagaagtagttaatgacaatactttagttgttctaatattatttatgtatggactcaaattaacatgcaaaacatatgagattagtggcatgcattctttttcttaatagtggaaaatacgagataatgataactgtgaagctctgttagtactcttcattactctatttgagtggcagcatatctcatgctagccataaagcaagttctagacgtattctgttgttaattacttgtagctatataacccaacctagtcattccagcttatgtctcttagagatcatgtttattagcacctcaagatttcctctgcacagtatagtaactatcgaaaaagatattatttctttgtttttaattgacaaccttcacgtgctacttatttttgcagcttggccggccaccaaccatggctgagatcaacaaccagaaccagtgcgtcccgtacaactgcctgagcaaccctaaggagatcatcctgggtgaggaacgcctggagaccggcaacaccgtagccgacattagcctgggcctcatcaacttcctctacagcaacttcgtgcccggcggtggcttcatcgtgggcctcctggagcttatctggggcttcatcggcccgtcccagtgggacatcttcctcgcccagatcgagcaactgatcagccagcggatcgaggagttcgctaggaaccaggccatctcccgcctggagggactctccaacctctacaaggtgtacgtgcgcgcgttcagcgactgggagaaggacccgaccaacccggccctccgcgaggaaatgcgtatccagttcaacgatatgaactcggccctcatcaccgccatcccgctcttccgcgtgcagaactacgaggtggccctcctgtccgtgtacgttcaagccgccaacctccacctctccatcctccgcgacgtgagcgtgttcggcgagcgctggggctacgacaccgccaccatcaacaaccgctactccgacctcacctccctcatccacgtttacaccaaccactgcgtggacacgtacaaccagggcctccgccgcctggagggccgcttcctctccgactggatcgtgtacaaccgcttccgccgccagctcaccatctccgtcctggacatcgtcgccttctttcccaactacgacatccgcacctaccctatccagaccgccacccagctcacccgcgaggtctacctcgacctcccgttcatcaacgagaacctcagcccggccgccgtctacccgaccttctccgccgctgagtccgccatcattcgcagcccgcacctcgtggacttcctcaactccttcaccatctacaccgactccctcgcccgcagcgcctactggggcggtcacctcgtgaactccttccgcaccggcaccactaccaacctcatccgcagcccgctctacggccgcgagggcaacaccgagcgcccggtgaccatcaccgccagcccgagcgtgcccatcttccgcaccctcagctaccccaccggcctggacaacagcaaccctgtggcgggcatcgagggcgtggagttccagaacaccatctccaggagcatctaccgcaagagcggccctatcgacagcttcagcgagctgcctcctcaggacgccagcgtgagccctgccatcggctacagccacaggctgtgccacgccaccttcctggagcgcatcagcggccctcgcatcgcgggcaccgtgttctcgtggacccaccgcagcgcctctcctacgaacgaggtgtctcctagtcgcatcacccagatcccttgggtcaaggcccacaccctggctagtggcgctagtgtcatcaagggccctggcttcaccggtggtgacatcctgaccaggaactctatgggcgagctgggcactctgagggtcactttcactggccgcctgcctcagtcttactacatccgcttccgctacgctagtgtcgctaaccgctctggtactttccgctactctcagcctccgtcttacggtatctctttccctaagactatggacgctggtgagcctctgaccagtaggagcttcgctcacactactctgttcactcctatcactttctctagggctcaggaggagttcgacctgtacatccagtctggtgtgtacatcgacaggatcgagttcatccccgtgaccgccacgttcgaggccgagtacgaccttgagcgcgcccagaaggtggtgaacgccctcttcactagcactaaccagctaggcctgaagactgacgtgaccgactaccacatcgaccaagtgagcaacctagtggcctgcctctccgacgagttctgcctcgacgagaagcgcgagctgtccgagaaggtgaagcacgccaagcgcctctccgacgagcgcaacctgctccaggaccccaacttcaggggcatcaacaggcagcccgaccgcggctggcgcggctccaccgacatcaccatccagggcggtgacgacgtattcaaggagaactacgttaccctccccggcaccttcgacgagtgttaccccacctacctctaccagaagatcgacgagtccaagctgaaggcctacacccgctaccagctccgcggctacatcgaggactcccaggacctggaaatctacctcatccgctacaacgccaagcacgagatcgtgaacgtgcctggcaccggcagcctctggcctctcagcgtggagaaccagatcggcccttgcggcgagcctaaccgctgcgcccctcacctcgagtggaaccctgacctccactgctcgtgcagggacggcgagaagtgcgcccaccatagccaccacttctctctggacatcgacgtgggctgcaccgacctgaacgaggacctgggcgtgtgggttatcttcaagatcaagacccaggacggtcacgccaggctgggtaacctggagttccttgaggaaaagcctctgctgggtgaggccctggccagggtcaagagggctgagaagaaatggagggataagagggagaccctgcagctggagaccactatcgtctacaaggaggctaaggagtctgtcgatgctctgttcgtcaactctcagtacgatagactgcaagctgataccaacatcgctatgatccacgctgcggataagcgggtccaccggatccgggaggcttaccttccggagctttctgtcatcccgggtgtcaacgctgcgatcttcgaggaacttgaggaacggatcttcactgcgtttagtctttacgatgcgcggaacatcatcaagaacggggacttcaacaatggtctgctgtgctggaacgtcaagggtcatgtcgaggtcgaggaacaaaacaatcatcgtagtgtccttgtcattcctgagtgggaggcggaggtctctcaagaggtccgtgtttgcccggggcgtgggtacattcttcgtgttactgcgtacaaggaggggtacggggaggggtgcgttactattcatgagattgagaacaatactgatgagcttaagttcaacaattgtgttgaggaggaggtttacccgaacaatactgttacgtgcatcaactacacggcaacgcaagaggaatacgaggggacgtacacctcgcgtaatagagggtatgatgaggcgtacggaaacaacccgtcggttccagcagattatgcctcggtttatgaggagaagtcgtacacggatagacgacgcgagaatccatgtgagtcaaatcgaggatacggagattacacaccattaccagcaggatacgttacaaaggagttggaatacttcccggaaacagataaagtttggattgaaatcggagaaacagaaggaacattcatcgtcgactcagtagaattgttgttgatggaagaatgatagggaccccggacccgccaaaaccattgcaaagactatagtttggggtggagtatacttggttgtgtacatgcctgcgtgttccattgtacacacaaaacctagccacctcttgactcttgagtgtatgcttgttatccgtgtgttgaagtttgtaagaggcaccatcactatagatgatggcttgtgtccctctttcatcaagattgaataatatatgctactttgagagcgctatcctgcttgcctgattgtgttaatacttacatccgtcccacactcccacaatataaggaaataaggtattttggcagtttagagcaaaattcccttatatttttgggacggatgtcctcttttctgcatttttttatgttcatatgttcctgaagagtaaggtggatcttgatcaacctgtcggtttatggtgattgatttgagtggaatagaatgggccaacgtccggcatacagttatgcttcagttaattaaaagtaagggtcccaagtaaggccggccaagtaacggtccgaagtaaggcgcgggtaccgtcggtccgggcctagtaggccaagcaggacgtggcgcgccaagaagaacgattggcaaacagctattatgggtattatgggtaggcacatgggaatatagtgggaggcagaggcgtgcctgtgcctgctcttaatttgagctcctcccctggccctgatagggcatgtgcatgcgcacaataatcattggagctgcctgaatgatttgcatgcgctcgtgtaaaacgttcacggtccatgccagtccaagaccgtcggcaagagatgagggccatgcgcagtacgtacatttgtatacatatacatatttttttggatgatgcgatgcgtgtgcatatctggctgcgccgctccctcgtccatcgtcgtcgcggtgtacaacgaattttccgtgggatgggctgaacttataatgggcctactgggcgggccgtatgaaatagcacacaattcaccctgcttttctcaccaggaatgcattttcgcgggagccatcgtcctaacttggcagacagacacacaaacacaggacaggagttcgccaaccccagcatgagagaaacctatccggacagctcctccagcctatgggccgttgggccttcagcctcagcgccccggccccgtgggcgggcaaaacactccacagtccacacggctgctgctgctgcccttgttccgtcggacctggggtaactttatagctatgatgttcgcccttcccaaccaatgtgcgagcgttaaggatgttaagtaacactagttaggattatgtggccaaatacctactttgctaggaacttgtacacccctcataatatagagaaaagaaagatgccagtggtccaactctataccatgtgtctcgtgtgtttgttatgtcgtgcttatgtgaagggagacgagtcttctacagcttctcacgcctaatctgttgacgggagagaagagagcggatctggtgatccgtggtaacgtagttctcaacaaatgacacattgagcacaccgttgtcatctcctcagctaaacattgagggggatttttttttaagaaaggctgagattcccttacgctccctttggcagggctcgtccaacagcttcttgaaaaattctataa

SEQ ID NO: 11 - DNA, 862 bp, Zea mays
  source 1..862: mol_type = genomic DNA; note = 862 nucleotide sequence representing the 5' flanking corn genomic DNA.
cgcagcgcaggtccgtgtggcctgtttgttttggcttctggtagcttctggccaccaaaagctgctgcggactgccaaacgctcagcttttcagccagcttctataaaatttgttggggggcaaaaaccatccaaaatcaacataaacacataatcagttgagtcgttgtaatagtaggaattcgtcactttctagatcctgagccctatgaacaattttatcttcctccacacgtaatcgtaatgatattcagattctccccacagccagattctcctcacagccagattttcagaaaagctggtcagaaaaaagctgaaccaaacaggccctaggacacacataggaatggatcaaaaaacatagcgccgtacccgtacgtacgtgcagtcacagtcagctgctagctagctagctgcgccggcggcggcgttgtcggtcttgactcaacgacgtccgcggctggcgccgtcggccagcggcttgttgccgccggtcccatccggcggagtccggctgccagtggccggcggcggcttcttcttcgtcgtcgtcctgccctttgccatccccgctgccacctggacgagggttctggctcccgtcgacgtcgacgagagacgtggcgccgcatttggctgccgccgtatcgtcgtcgtcgtcgagattgaaatcgagagggagcgcgcgggatggctgccgccggtggcgcgacatggtctcgcccggccggcgcctgatcgacttctcttccgccgccttcgctagctatagcttggtagtagtcaccaacaacgacctagggagctagcgtacgttcgaggtacctgtcctgcaagcaaatgtgtggtagtggtccaa

SEQ ID NO: 12 - DNA, 1036 bp, Zea mays
  source 1..1036: mol_type = genomic DNA; note = 1036 nucleotide sequence representing the 3' flanking corn genomic DNA.
acatgggaatatagtgggaggcagaggcgtgcctgtgcctgctcttaatttgagctcctcccctggccctgatagggcatgtgcatgcgcacaataatcattggagctgcctgaatgatttgcatgcgctcgtgtaaaacgttcacggtccatgccagtccaagaccgtcggcaagagatgagggccatgcgcagtacgtacatttgtatacatatacatatttttttggatgatgcgatgcgtgtgcatatctggctgcgccgctccctcgtccatcgtcgtcgcggtgtacaacgaattttccgtgggatgggctgaacttataatgggcctactgggcgggccgtatgaaatagcacacaattcaccctgcttttctcaccaggaatgcattttcgcgggagccatcgtcctaacttggcagacagacacacaaacacaggacaggagttcgccaaccccagcatgagagaaacctatccggacagctcctccagcctatgggccgttgggccttcagcctcagcgccccggccccgtgggcgggcaaaacactccacagtccacacggctgctgctgctgcccttgttccgtcggacctggggtaactttatagctatgatgttcgcccttcccaaccaatgtgcgagcgttaaggatgttaagtaacactagttaggattatgtggccaaatacctactttgctaggaacttgtacacccctcataatatagagaaaagaaagatgccagtggtccaactctataccatgtgtctcgtgtgtttgttatgtcgtgcttatgtgaagggagacgagtcttctacagcttctcacgcctaatctgttgacgggagagaagagagcggatctggtgatccgtggtaacgtagttctcaacaaatgacacattgagcacaccgttgtcatctcctcagctaaacattgagggggatttttttttaagaaaggctgagattcccttacgctccctttggcagggctcgtccaacagcttcttgaaaaattctataa

SEQ ID NO: 13 - DNA, 18376 bp, synthetic construct
  source 1..18376: mol_type = other DNA; note = Sequence representing the transgene cassette comprised within the binary plasmid transformation vector used to transform corn to produce corn event MON 95379.
aaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcgatgcggccaccacgagtcgacataacttcgtatagcatacattatacgaagttatactagttggtgttttctgttacagagcaactttatcatagttaggaacttgggtgtggctctagccgctcccagtcgaatgtttctaaattaaaaatccaaacaaaaatcactgaggtttacttgggacacaatctttacacagctacgtattcacgcgttctcttttcacacagctacgtattctggtgaggagcatttacttaggaacatcacttagctcacctaagcagcgacgatctcaagattgcgaaccagagaatcagatatccaaaactagtttcacgccaactaaaggtcacaattattctcgagcaaaaaaacgatttcaatgctatagacacaactttacgaagtagaggcttcgttttacgacaaaccacgaacttggatattaaagatgccacaaagaaggttcacataaacaacacaggtcacagcagcgaaagctacaagcaaaccctacataatcgaaaagcaaatgtttttcaaaaccagacacacaaactgatagatagaagcccacacggacagatcataggatagcagcagtaatcaagcattgccaaggcaccaggcaagaaccctggagctcatcaggcagccttcgtatcggagagttcgatcttcgcgcccagcccggccatcaggtccatgaactccgggaagctcgtggcgatcatcgtggcatcgtccaccgtgacagggttttccgacacgaggcccatgacgaggaagctcatggcgatgcggtgatcgagatgggtggcgacggcggcgcccgaggcgttgccgagccccttgccgtcagggcggccacgcacgacgagcgacgtctcgccctcatcgcaatccacgccattgagcttgaggccattggcgacggccgagaggcggtcgctttccttgacgcggagttcttccagaccgttcatcacggtcgccccttccgcgaaggcggcggcgacagcgagaatcggatattcgtcgatcatcgaaggcgcgcggtcttccggcaccgtgacgcccttcagcgtggaggagcgaacgcgcaggtccgccacgtcttcgccgccggcaaggcgcgggttgatgacttcgatgtcggcgcccatttcctgcagcgtcaggatgaggccggtgcgggtggggttcatcagcacgttgaggatggtgacgtcggagcccggaacaagcagggccgcaaccagcgggaaggccgtcgaggacgggtcgcccggcacgtcgatgacttggccggtgagcttgccgcggccttccaggcggatggtgcgcacgccgtccgcatccgtctcgacggtaaggttggcgccaaagccctgcagcatcttttccgtatgatcgcgcgtcatgatcggctcgatgaccgtcgtgatgccgggcgtgttgaggccggcgagcagcacggcggacttcacctgtgcggaggccatcggcacgcggtaggtgatcggcgtcggcgtcttcggcccgcgcaaggtaacgggaagacggtcaccgtcttccgatttcacctgcacgcccatttcgcgcagcgggttcaacacgcggcccatcgggcgctttgtgagcgaggcgtcgccgatgaaggtgctgtcgaaatcgtagaccccgacgaggcccatcgtcaggcggcagcccgtggcggcattgccgaaatcgagcggcgcctcaggcgccaggaggccgccattgccgacgccatcgatgatccaggtgtcgccttccttacggatgcgggcgcccatcgcctgcatggccttgcccgtattgatgacgtcctcgccttccagaaggccggtgatgcgcgtttcaccgctcgcgagaccgccgaacatgaaggaccggtgggagatcgacttgtcgccgggaatgcggacggttccggaaaggccagaggatttgcgggcggttgcgggccggctgcttgcaccgtgaagcatgcacgccgtggaaacagaagacatgaccttaagaggacgaagctcagagccaattaacgtcatcccactcttcttcaatccccacgacgacgaaatcggataagctcgtggatgctgctgcgtcttcagagaaaccgataagggagatttgcgttgactggatttcgagagattggagataagagatgggttctgcacaccattgcagattctgctaacttgcgccatggatcaggctgcataggacaaaacaagatttcaacaaattacgccttaagaattttttttatcacaggatttatcaagacaagacgaacagggagtcacaggtgaaccaatccacataatacagcctttcagatagcgttctaatgacctttaagaatataatagcacctatcttcagatcagatcaaaagattactacgcagaaacgagcactacaaagcatgataccaaagaccatcaaatagttgcccaatctgtctaaatatttttttctgtgcttgctctgaaatagttcttagctcgcagcaatgtacatactacagatccacatactccgtccatacatgtgcctaagttacagatctattcataataagcaacaaattaagtataaacgggagtacaaatctaggtgaacagcagtaaaatatcaagatccatcaaccaaaggggcatttcagcacggatcatggcaatccaaccagatccgcatgccattcctatacaacctaggtagaaaatttgaaaaaacaaacaagcattgatcagatctatccgaaacaaacatacactaaccagatctaccaaaaaggaacagattatcagcagggagacagactaattcagtggatccccatccgaaacccccctaatattgcatcatttcccgattcttagcgtctcactgccagatccaaaacatttcgccagatccacgaactaccactgatcaaaacgcagatccggacatcgacaaccacagaaggccagatccaccgacagatctatcaaacagaacgaaccacaccccaggatcacccagatccacccacagatcgaagcgcgctactacgagaaacggaggaatggaagcatcagatccgtttttgtttgttttacctggatggctagccggtggcggcggagacgaacggcggcgagcggcggcggaggagggcgcggagagaggcgagtacgaagacgcctttctctgcggtgtggtagggtttccacctcccactcctcccctttctatagagcgggaggcgcctcggcgtttttgcgtttaaccccctcgattaagtttcccaattttacactccgctccactttggctcctccaacggtcaggaactgtactggtttggaatatcggaatttttctgagggttttagtgcaaaatattcatgccgttttgtttagtgtgggtgggtaagagtccagatctacccgccgctcactgggaggtgggcccactggttgctgcgagcgactgacgtctcgcttgtaggtgggcccacaagggtgggaccggattgtcagtggcgtgtgggttggtgggcgtagttggggtagaattcgaggggttgttccgtcattttccgcccgctccgtcggcggccagtcgtgccgatatttttggattttgaaattcaagagcgggagccaggagggggagggtggtggcaggcctggcccatgatctattgtttgctcggatgggccttgggcctaactgttagttttgattctgtttgtaattaactctttttgaaaggaataaaaagaaccatgctacctgacgggttgatgttttaccattgagatttcaaaggggttaaagtgaaattgaaactcaattcgaacatgattttaattgttttgaactcccttcgaattgagggcagttttagtggttataaatcttcatcgagagaatttttctgatgtttttatggaatttgatgtcctaagagcacctctaacaggcaacatcctctccaaatggctctcaaactatttttttttgacaattctagtaaaaagatactaccccaataacctttccaaccggatggccaaatccctccccttattggcctaacttggcaacccaaaataacttgccaaacggtaggccccatgtactattcctcctccccacatggctactctcctcttctctctctctcttatgttgacggagaggaggcgttgacggtggctgttgggcgaagctagggtagtgcggtggtggaggtgaagcggtggctgtgctaaagctcgcccctggcattggtggtgtgggagagctggccagtggctagtgtgacaatggaggtgaccaggcgacactttctccctccctcttccctccatgttgatgagaagcatgttgttgtccctaggataacttcgtatagcatacattatacgaagttatagcggccgcaaatcaacctcactctatttaaatgaggtggtaggatttgccgcgtgagacctattaccctgttatccctagtttaaacacctgcaggaaaagattaattaacatcgatcatcatgttcaattgacagcaaattaaactagttatatatagggttcatgagatgagagtagctaattattaacacaagtggaagaaacttttatttgacggtatacaaataacaagatacagagatcaggctgatggatcaatcatatcatatcagtgatagatcgatcgttcgacacatatatatgtatgacaaaacatagtataagtacgtgcgaaagggtttatatatatatggatattgatgcatggtgtaatggtatggtaattaagggatcggaggatcgattaatgggtccctatcactcctccatcagtagcagctcgaccgagtccacgatgaaagtgccctccgtctcgccgatctcgatccagactttgtcggtctccgggaagtactccagctccttggtgacgtacccggcgggcagcggagtgtagtcaccgtagccacggttggactcgcaagggttgtccctccgtccgtcggtgtacgccttctcctcgtaagcggacgcataatccgcaggcacgctgctgttggactcgtacgcgccatcatagccgcgattccgggaagtgtaagtgccctcgtactcctcttgcgtggcagtgtagtcgttgcaagtaacggtgttgttcgggtacacctcctcctccacacagttcgagaacttgagctcgtcggtgttgttctcgatctcgtggatagtcacgcagccttcaccgtagccttccttgtacgcagtcacacggaggatgtagccgcgaccagggcagacgcggacctcctggctcacctctgcctcccactccggcacgaccagcacggaccggtggttgttctgctcctcgacgtccacatggcccttgacgttccagcaactcagcccgttgttgaagtcaccgttcttaataacattcctggcatcgtacaggctgaatgctgtgaagatgcggccctccagttcctcgaagatcgccgcgttcacaccagggatcactgacagctcgggcaggtacgcctcacggatgctgtggacccgcttgtcagccgcgtggatcatggcgatgttggtgtcagcctggaggcggtcgtactggctgttgacgaagagtgcgtccactgactccttggcctccttgtacacgatgttggtctcccactccagcttctcccgcttgtctcgccatttcttctcggcgcgcttcacacgagcgagggcctcgcccacgagaggcttctcctcaaggaactccaggttgccgaggcgggcatgtccgtcctgggtcttaatcttgaagataacccagacacccaggtcctcgttgaggtcggtgcagcccacgtcgatgtccagagagaagtggtgcgagtggtgagcgcactttccgattggagagggagcgctcaatggccaaagtgagcccgtgcctggcacgttcactgtctcgtgcttggcgttgtatcggatcaggtagatttccaggtcctgactatcctcgatgtacccgcgaagctggtagcgggtgtacgccttcaatttactctcgtctatcttctggtagagatacgtcgggtagcattcgtcgaaagtcccgagcagcgtcacatagttctccttgaacacatcgtcgcctccctggatggtgatgtctgtactcccgcgccaaccacggtcgagctggcggttgatgccacggaagttcgggtcctggagcaaattccgctcgtcactcagacgcttcgcgtgtttgaccttctcggacagctccttcttctcgtcgaggcagaactcgtcggacaggcactccacaaggttggacacttggtcaatgtgatagtccgtgacgtcggtcttgagcccaatctggttgctgctggtgaacagttcgttgacagccttctgagcccgctccaggtcagactcggcctcgaacgtcgcatccgccaggatgatctcaatcttgtcgatgtacagttcgccagagctgatggagccagcaccgaacagcggttgctcggagatcccaatgatgtcagggttcgctctgaatgagaaagggttcgagaaatcagtgtaccggaaggttcggctagtcaagttctcgcctatctccatcgtcttctggagaggcatgttgactgagacttggcctcccacgcctgtggacgcggctccggtcaggacaatgaccctcgcgtcgcggcttgaagcgtagcggaacctcagacggtaacgctgggtgatcggcgagttgatgttcacttgcaacgaaacgaagtcgccgaaagtgttgcgccgcagtatgtctccaccggtgaagcccggaccggtgatgacgctcgtgccaccccacacacggaagcccttcacgagcgggatctggttaatggagtcggaagagatcgtgttggtccggtcggcgctgcggtgtgtccaggagtacaccggcgcacgcagcgtgttgccagagatgagccggatgttggacaggcggtggctgtaggactcgtagttcgggcgttccgtggtctcgggcggcagctcagtttcactgtcgaagagctgggtgccgacgcccgtgtacccgatggtgtagagcaatgacccgcgcagggagttcaacgggttacgccagttgaaccgggcccacgggacgccgttgacgggcgttgtcagaaggatgttaatgcccgcgaacgactcagtccggtaaacgtcgcggctcgtgaactgtagcgtaactgggttgatggaggtgttggtgttgccgtgggtactggtgctcaagctgccacggatcgtcctactctccaatcggtgcccgacccagtagttcatgtactgtgtgttggaccagcgtgagagcacggagaagatcgtgagctgctcgggaaagtcgagcaggtgaggagggcggatgacggcggcctcgattgccgagaaggagggtgcgttgttgttgaaccaattcgtggatgcgaaaccggagggtgcgttcgtccggccgattgggtctgtgtagatctcccgcgtgagttgcgcgctagtgttcattgggtacacccgtgtgtcgtaagacgggaatagcgccaccaggtctaggacacccaaagtcaagtcgcggcggaactggttgtaacggagccatgactcagcgttggtgccgcgcaggttgttgagccctgtgttgtaccatcgagcgcagtagtcactgtactcacgggtcttctccacttggcgctcgtagtaacgctggatttcttgagatgtgagtccgaactcggagccgaacaggctggcgtcccgcagcaggagcagatgtaagttggcggcctgggcgtacaccataaggagtggaacttcctggttcctgatggcgaacagcggcatagcgttcaggaagtcgagttcgagagcgatgtactgggtgtagagtaccgagcgtgttcgcgcgtcatccctgttctccaaccagtcctccagtgactgctggtatgcccgaaaggagtttcccaggccctgtagcctggccagagcagtatcgcgggtgttctccgtgacttgctgccggatcaactgctccacatgctccaggaagatctcccacgggtcacgaccgcgaggccacagctcgcccacaaggaacgagtagaaggacgcgatctgaccggcgaatgggacaccgaggacgccgaggatacggcccgcgatgttgatgccggtctgaaccgtgctggccgacacgaatgggtcgatgttgttgccctcggctatgcagagggagtcctcgatgcgcgcgtcggtggacaggttcatctgcgcggagtggtttgaaacagcagggatcgagagggcgttgatgatctcgttctcgttcttccggttgctcgtcatggtggtggccggccaagtaacggtccgacctgcagaagtatcaccaaacaacagggtgagcatcggacaaaagaaacagtaccaagcaaataaatagcgtatgaaggcagggctaaaaaatccacatatagctgctgcatatgccatcatccaagtatatcaagatcaaaataattataaaacatacttgtttattataatagataggtactcaaggttagagcatatgaatagatgccgcatatgccatcatgtatatgcatcagtaaaacccacatcaacatgtatacctatcctagatcaatatttccatccatcatcttaaactcgtaactatgaagatgtatggcacacacataaagatacaaaattaataaatccaccaggtagtttgaaacagtattctactccgatctagaacgaccgcccaaccagaccacatcatcacaaccaagcgaaaaaaagcatctctgtatatgcatcagtaaaacccgcatcaacatgtatacctatcctagatcgatatttccatccatcatcttaaactcgtaactatgaagatgtaggcacatacatacagatcctttaataaatccaccaggtagtttgaaacagtattctactccgatctagaacgaccctttatcctttaataaatccaccaggtagtttgaaacagtattctactccgatctagaacgaccgcccaaccagaccatatcatcacaaccaagcgagaaaaaaagcatctctgtatatgcatcagtaaaacccgcatcaacatgtatacctatcctagatcgatatttccatccatcatcttaaactcgtaactatgaagatgtatggcacacacatacagatacaaaattaataaatccaccaggtagcttgaaacagtatcctactccgatctagaacgaccgcccaaccagaccacatcatcacaaacatgaacatgaacatgtttgctctaacacaaacatgaacagaagtagaactaccgggccctaaccatggaccggatcgccgatctagagaaggtagagagagggggggggggaggatgagcggcgtaccttgaagcggaggtgccgacggctggatttgggggagatctggttgcgtgtgtgtgcgctccgaacgaacacgaggttggggaaagagggtgtggagggggtgtctatttattacggcgggcgaggaagggaaagcgaaggagcggtgggaaaggaatcccccgtagctgccggtgccgtgagaggaggaagaggccgcctgccgtgccgcctcacgtctgccgctccgccacgcaatttctggatgccgacagcggagcaagtccaacggtggagcggaactctcgagaggggtccagaggcagctacagagatgccgtgccgtctgcttcgcttggcccgacgcgacgctgctggttcgctggttggtgtccgttagactcgtcgacggcgttgaacagcctgtcattatctactcgaaacaagaaaaatgtttgcttagtttttttatttcttaaagggtatttgttttatttgtagtcaatttattttatttcattttatatctaaattattaaataaaaaactaaaatagagttttagttttaaaaaatttagagactaaaaagaataaaatggatgtactaaaaattagtctatagaaaccatcaaccctaaatcctaaatggatgtactaataaaatggatgaagtattatataggtcaagctatttgcaaaaaaaacagagagatcacatgcacactaaaaagataaaactgtagattgtcaaaatattcaattatcctttagaccatgtctagcagtttatttatatgatcctctaaaacactaatattattttagtattatagactatattatttgtagagtgaagtttaaatatatgtatagagatagataaactacacttcaaataagtgtgacaaaaaaatatgtggtaattttttatactttagacatgcaatgctctttatctctagagaggggcacgacgcatgcaagcttgttaacgcggccgctaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattagcttgagctcaggatttagcagcattccagattgggttcaatcaacaaggtacgagccatatcactttattcaaattggtatcgccaaaaccaagaaggaactcccatcctcaaaggtttgtaaggaaggtcgaggccgctgtactgtcatattgtcgtggtttttcaattgctgtacctgatgcaaacgtaatgggtttactaatcttgcacccgccggcttcaaaatgaagagtgctaatttggtccacgtcaccatcaccggttcgaactgtctagaatggcaggcaaagatgattggacaggcatgcagggaaaaagagcaccgttgacgatgtatgcgagttcccaccattgcgagcaatgattatcagccacacgacttactcttcagagctaaccactgccatgcagagaaaaagtgaatcatattgtcatgatctacaacgaagtgaaacaatcaggcatgctaaagtgctgaaactttactgatctctcatgttggacaacaaagaatacgggaatacatcagcaacgcaactcttgagctttgcttgccgaatgaccagctagaatttccaagcatttacagaaacatgactttaagtttcagaaaaacaaatacaaggccactaaataagcgtggggataacatatcctccagatgacaggcaatctgcaacttgcagccattcaaatgtacgattaacaaaatatttaagcgccacatgagataatatatcctccaattagggcctttagtattgtcattagctcataaccatggtgcatcctcacatggacgctgcataagaagttcataatagcaacagacatatgaacaaagcatggtgcgcctgcccggccggactagctagtactaccaatcatggaataagctagtaccctaaatgaaattaaaatggtttttagcgattatccacgccgtccagaatactctaatccacaagttgaggccgcccatgaagccgcgagagggcgacgccatgtgtataaaaggggcctaagctgagtggacttgctgcatcagattagtaagcaatctcaagcgcagagagccaaagctttcggtgtagctcgaagagcaaagcgaaggcaaggcgcgcctgtaacctacctaccaacctcgccattcctctccaaactgttgtgctgctgtctagatctcccacactacactagttactcctcgtagatctcggctacctggctcaagatccggggtcagatccgggtccggggattttctttgtgccctatggctgtattttggcgtctgtggctgatgacagcgtgtgttctcgagtgcggatgcaatctgagttatataggcaaatggccttgtcaactcgggcagcggcattgctttgctcagtgtgtttgaatgtgctgaaattcatgtagtaggctgtaggctgtgcatttcttgatttgcgtcttgcataattcactggtggattttctaaacctaacaagtttaaaattagaccattcaaccaaagacaggaggaataagtgaagctgttgtagtcacagcttatggccgatccaaaatttgttaggaatgtgaatatgtgatgctacaaacatatccttgtaagctaccatgctatttatcatgttccatcatggtgattggtgagcactcatgaaaatttcagatccaaacctagtgttacatgtggatttgtgctctgcaatctatcgccagtaataaaatggttgagtgatccagctactacaaaatcacattgcatactttttttttttgtagattatgcatcctggttttgggtggtgggttcctgatgtcaggaatataaatttagcctgctgatttaggtagcactgccggtgcacactttggtttttgaatacttgtagtcttccagcttcttgtagaactggtacaatgtgggccatatataagaagggctgtcaactagcacatgctcactaattagtctaaacatttatgtttttattcattcaggtcaggtgcaatcatagaagtagttaatgacaatactttagttgttctaatattatttatgtatggactcaaattaacatgcaaaacatatgagattagtggcatgcattctttttcttaatagtggaaaatacgagataatgataactgtgaagctctgttagtactcttcattactctatttgagtggcagcatatctcatgctagccataaagcaagttctagacgtattctgttgttaattacttgtagctatataacccaacctagtcattccagcttatgtctcttagagatcatgtttattagcacctcaagatttcctctgcacagtatagtaactatcgaaaaagatattatttctttgtttttaattgacaaccttcacgtgctacttatttttgcagcttggccggccaccaaccatggctgagatcaacaaccagaaccagtgcgtcccgtacaactgcctgagcaaccctaaggagatcatcctgggtgaggaacgcctggagaccggcaacaccgtagccgacattagcctgggcctcatcaacttcctctacagcaacttcgtgcccggcggtggcttcatcgtgggcctcctggagcttatctggggcttcatcggcccgtcccagtgggacatcttcctcgcccagatcgagcaactgatcagccagcggatcgaggagttcgctaggaaccaggccatctcccgcctggagggactctccaacctctacaaggtgtacgtgcgcgcgttcagcgactgggagaaggacccgaccaacccggccctccgcgaggaaatgcgtatccagttcaacgatatgaactcggccctcatcaccgccatcccgctcttccgcgtgcagaactacgaggtggccctcctgtccgtgtacgttcaagccgccaacctccacctctccatcctccgcgacgtgagcgtgttcggcgagcgctggggctacgacaccgccaccatcaacaaccgctactccgacctcacctccctcatccacgtttacaccaaccactgcgtggacacgtacaaccagggcctccgccgcctggagggccgcttcctctccgactggatcgtgtacaaccgcttccgccgccagctcaccatctccgtcctggacatcgtcgccttctttcccaactacgacatccgcacctaccctatccagaccgccacccagctcacccgcgaggtctacctcgacctcccgttcatcaacgagaacctcagcccggccgccgtctacccgaccttctccgccgctgagtccgccatcattcgcagcccgcacctcgtggacttcctcaactccttcaccatctacaccgactccctcgcccgcagcgcctactggggcggtcacctcgtgaactccttccgcaccggcaccactaccaacctcatccgcagcccgctctacggccgcgagggcaacaccgagcgcccggtgaccatcaccgccagcccgagcgtgcccatcttccgcaccctcagctaccccaccggcctggacaacagcaaccctgtggcgggcatcgagggcgtggagttccagaacaccatctccaggagcatctaccgcaagagcggccctatcgacagcttcagcgagctgcctcctcaggacgccagcgtgagccctgccatcggctacagccacaggctgtgccacgccaccttcctggagcgcatcagcggccctcgcatcgcgggcaccgtgttctcgtggacccaccgcagcgcctctcctacgaacgaggtgtctcctagtcgcatcacccagatcccttgggtcaaggcccacaccctggctagtggcgctagtgtcatcaagggccctggcttcaccggtggtgacatcctgaccaggaactctatgggcgagctgggcactctgagggtcactttcactggccgcctgcctcagtcttactacatccgcttccgctacgctagtgtcgctaaccgctctggtactttccgctactctcagcctccgtcttacggtatctctttccctaagactatggacgctggtgagcctctgaccagtaggagcttcgctcacactactctgttcactcctatcactttctctagggctcaggaggagttcgacctgtacatccagtctggtgtgtacatcgacaggatcgagttcatccccgtgaccgccacgttcgaggccgagtacgaccttgagcgcgcccagaaggtggtgaacgccctcttcactagcactaaccagctaggcctgaagactgacgtgaccgactaccacatcgaccaagtgagcaacctagtggcctgcctctccgacgagttctgcctcgacgagaagcgcgagctgtccgagaaggtgaagcacgccaagcgcctctccgacgagcgcaacctgctccaggaccccaacttcaggggcatcaacaggcagcccgaccgcggctggcgcggctccaccgacatcaccatccagggcggtgacgacgtattcaaggagaactacgttaccctccccggcaccttcgacgagtgttaccccacctacctctaccagaagatcgacgagtccaagctgaaggcctacacccgctaccagctccgcggctacatcgaggactcccaggacctggaaatctacctcatccgctacaacgccaagcacgagatcgtgaacgtgcctggcaccggcagcctctggcctctcagcgtggagaaccagatcggcccttgcggcgagcctaaccgctgcgcccctcacctcgagtggaaccctgacctccactgctcgtgcagggacggcgagaagtgcgcccaccatagccaccacttctctctggacatcgacgtgggctgcaccgacctgaacgaggacctgggcgtgtgggttatcttcaagatcaagacccaggacggtcacgccaggctgggtaacctggagttccttgaggaaaagcctctgctgggtgaggccctggccagggtcaagagggctgagaagaaatggagggataagagggagaccctgcagctggagaccactatcgtctacaaggaggctaaggagtctgtcgatgctctgttcgtcaactctcagtacgatagactgcaagctgataccaacatcgctatgatccacgctgcggataagcgggtccaccggatccgggaggcttaccttccggagctttctgtcatcccgggtgtcaacgctgcgatcttcgaggaacttgaggaacggatcttcactgcgtttagtctttacgatgcgcggaacatcatcaagaacggggacttcaacaatggtctgctgtgctggaacgtcaagggtcatgtcgaggtcgaggaacaaaacaatcatcgtagtgtccttgtcattcctgagtgggaggcggaggtctctcaagaggtccgtgtttgcccggggcgtgggtacattcttcgtgttactgcgtacaaggaggggtacggggaggggtgcgttactattcatgagattgagaacaatactgatgagcttaagttcaacaattgtgttgaggaggaggtttacccgaacaatactgttacgtgcatcaactacacggcaacgcaagaggaatacgaggggacgtacacctcgcgtaatagagggtatgatgaggcgtacggaaacaacccgtcggttccagcagattatgcctcggtttatgaggagaagtcgtacacggatagacgacgcgagaatccatgtgagtcaaatcgaggatacggagattacacaccattaccagcaggatacgttacaaaggagttggaatacttcccggaaacagataaagtttggattgaaatcggagaaacagaaggaacattcatcgtcgactcagtagaattgttgttgatggaagaatgatagggaccccggacccgccaaaaccattgcaaagactatagtttggggtggagtatacttggttgtgtacatgcctgcgtgttccattgtacacacaaaacctagccacctcttgactcttgagtgtatgcttgttatccgtgtgttgaagtttgtaagaggcaccatcactatagatgatggcttgtgtccctctttcatcaagattgaataatatatgctactttgagagcgctatcctgcttgcctgattgtgttaatacttacatccgtcccacactcccacaatataaggaaataaggtattttggcagtttagagcaaaattcccttatatttttgggacggatgtcctcttttctgcatttttttatgttcatatgttcctgaagagtaaggtggatcttgatcaacctgtcggtttatggtgattgatttgagtggaatagaatgggccaacgtccggcatacagttatgcttcagttaattaaaagtaagggtcccaagtaaggccggccaagtaacggtccgaagtaaggcgcgggtaccgtcggtccgggcctagtaggccaagcaggacgtggcgcgccaagaagaacgattggcaaacagctattatgggtattatgggtaggcctgcccgggctcagcctaagtcgctaccttaggaccgttatagttaccgggaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcct

SEQ ID NO: 14 - DNA, 35 bp, unidentified
  source 1..35: mol_type = other DNA; note = Enterobacteria phage P1; note = 35 nucleotide sequence representing the LoxP sites used for Cre-mediated excision and recombination.
ataacttcgtatagcatacattatacgaagttata

SEQ ID NO: 15 - DNA, 20 bp, synthetic construct
  source 1..20: mol_type = other DNA; note = 20 nucleotide sequence corresponding to a thermal amplification primer referred to as SQ51219 used to identify corn event MON 95379 DNA in a sample.
cctgcaagcaaatgtgtggt

SEQ ID NO: 16 - DNA, 30 bp, synthetic construct
  source 1..30: mol_type = other DNA; note = 30 nucleotide sequence corresponding to a thermal amplification primer referred to as SQ21524 used to identify corn event MON 95379 DNA in a sample.
gtcgttttatcaaaatgtactttcatttta

SEQ ID NO: 17 - DNA, 16 bp, synthetic construct
  source 1..16: mol_type = other DNA; note = 16 nucleotide sequence corresponding to a probe referred to as PB10269 used to identify corn event MON 95379 DNA in a sample.
taacgctgcggacatc

SEQ ID NO: 18 - DNA, 24 bp, synthetic construct
  source 1..24: mol_type = other DNA; note = 24 nucleotide sequence corresponding to a thermal amplification primer referred to as SQ20222 used as an internal control for the event and zygosity assay for MON 95379 and hybridizes to a region of the corn genome.
gccctatgacttaccgagagttca

SEQ ID NO: 19 - DNA, 28 bp, synthetic construct
  source 1..28: mol_type = other DNA; note = 28 nucleotide sequence corresponding to a thermal amplification primer referred to as SQ20221 used as an internal control for the event and zygosity assay for MON 95379 and hybridizes to a region of the corn genome.
gttgctatgtactaacagaactgcatgt

SEQ ID NO: 20 - DNA, 29 bp, synthetic construct
  source 1..29: mol_type = other DNA; note = 29 nucleotide sequence corresponding to a probe referred to as PB50237 used as an internal control for the event and zygosity assay for MON 95379 and hybridizes to a region of the corn genome.
ttgttgtgtggctccattctgacttgtga

SEQ ID NO: 21 - DNA, 20 bp, synthetic construct
  source 1..20: mol_type = other DNA; note = 20 nucleotide sequence corresponding to a thermal amplification primer referred to as SQ50998 used in the zygosity assay for event MON 95379 and hybridizes to the coding sequence of Cry1B.868.
cgggtagcattcgtcgaaag

SEQ ID NO: 22 - DNA, 20 bp, synthetic construct
  source 1..20: mol_type = other DNA; note = 20 nucleotide sequence corresponding to a thermal amplification primer referred to as SQ50997 used in the zygosity assay for event MON 95379 and hybridizes to the coding sequence of Cry1B.868.
ggcgacgatgtgttcaagga

SEQ ID NO: 23 - DNA, 18 bp, synthetic construct
  source 1..18: mol_type = other DNA; note = 18 nucleotide sequence corresponding to a probe referred to as PB50340 used in the zygosity assay for event MON 95379 and hybridizes to the coding sequence of Cry1B.868.
aactatgtgacgctgctc

SEQ ID NO: 24 - DNA, 19 bp, synthetic construct
  source 1..19: mol_type = other DNA; note = 19 nucleotide sequence corresponding to a thermal amplification primer referred to as SQ50485 used in the zygosity assay for event MON 95379 and hybridizes to the coding sequence of Cry1Da_7.
ggcttaccttccggagctt

SEQ ID NO: 25 - DNA, 18 bp, synthetic construct
  source 1..18: mol_type = other DNA; note = 18 nucleotide sequence corresponding to a thermal amplification primer referred to as SQ50484 used in the zygosity assay for event MON 95379 and hybridizes to the coding sequence of Cry1Da_7.
cgaagatcgcagcgttga

SEQ ID NO: 26 - DNA, 14 bp, synthetic construct
  source 1..14: mol_type = other DNA; note = 14 nucleotide sequence corresponding to a probe referred to as PB50138 used in the zygosity assay for event MON 95379 and hybridizes to the coding sequence of Cry1Da_7.
acccgggatgacag

SEQ ID NO: 27 - DNA, 21 bp, synthetic construct
  source 1..21: mol_type = other DNA; note = 21 nucleotide sequence corresponding to a thermal amplification primer referred to as PNEGDNA used in the zygosity assay for event MON 95379.
ccactgcgcgcgcgccatggt

SEQ ID NO: 28 - DNA, 14 bp, synthetic construct
  source 1..14: mol_type = other DNA; note = 14 nucleotide sequence corresponding to a probe referred to as PRBNEGDNA used in the zygosity assay for event MON 95379.
cgtcctccgatcca